(19) United States
(12) Patent Application Publication
(10) Pub. No.: US 2025/0154272 A1
(43) Pub. Date: May 15, 2025

(54) ANTI-CLDN4/ANTI-CD137 BISPECIFIC ANTIBODY

(71) Applicants: Astellas Pharma Inc., Tokyo (JP); National Cancer Center, Tokyo (JP)

(72) Inventors: Yoshiyuki TENDA, Tokyo (JP); Masatoshi YURI, Tokyo (JP); Shigenori YAGI, Tokyo (JP); Yoshiki SATAKE, Tokyo (JP); Kazunori HIRAYAMA, Tokyo (JP); Hiroki SHIRAI, Tokyo (JP); Hiroki SASAKI, Tokyo (JP); Fumiko CHIWAKI, Tokyo (JP); Masayuki KOMATSU, Tokyo (JP)

(73) Assignees: Astellas Pharma Inc., Tokyo (JP); National Cancer Center, Tokyo (JP)

(21) Appl. No.: 19/020,859

(22) Filed: Jan. 14, 2025

Related U.S. Application Data
(63) Continuation of application No. 18/556,606, filed on Oct. 20, 2023, now Pat. No. 12,227,586.

(30) Foreign Application Priority Data
Apr. 22, 2021 (JP) 2021-072429

(51) Int. Cl. C07K 16/28; A61K 39/00; A61P 35/00

(52) U.S. Cl. CPC C07K 16/2878; A61P 35/00; C07K 16/28; A61K 2039/505; C07K 2317/31; C07K 2317/622

(57) ABSTRACT

An object of the present invention is to provide an anti-CLDN4/anti-CD137 bispecific antibody usable in treatment of cancer. An anti-CLDN4/anti-CD137 bispecific antibody produced using an anti-CLDN4 antibody binding to CLDN4 and an anti-CD137 antibody binding to CD137 had agonistic activity for CD137, promoted production of interferon γ by a T cell, and exhibited cytotoxic activity against a cancer cell expressing CLDN4 on a cell surface thereof. Besides, it was shown that the anti-CLDN4/anti-CD137 bispecific antibody can be safely administered to monkeys. Therefore, the anti-CLDN4/anti-CD137 bispecific antibody is usable in treatment of human cancer.

### CROSS-REFERENCE TO RELATED APPLICATIONS

This application is a Continuation of U.S. application Ser. No. 18/556,606, which is the U.S. National Stage of PCT/JP2022/018350, filed Apr. 21, 2022, which claims priority to JP2021-072429, filed Apr. 22, 2021.

### SEQUENCE LISTING

The instant application contains a Sequence Listing which has been submitted electronically in XML format and is hereby incorporated by reference in its entirety. Said XML copy, created on Jan. 13, 2025, is named 050499-0120_SL.xml and is 118,866 bytes in size.

### TECHNICAL FIELD

The present invention relates to an anti-CLDN4/anti-CD137 bispecific antibody expected to be useful as an active ingredient of a pharmaceutical composition for use in treatment of cancer.

### BACKGROUND ART

Claudin-4 (CLDN4) is a tetraspan membrane protein belonging to the claudin family. It is expressed in an epithelial cell and an endothelial cell, and plays a significant role as a main molecule contained in a tight junction. CLDN4 is highly expressed in a cancer tissue of colorectal cancer, bladder cancer, ovarian cancer and the like, and it has been suggested that an anti-CLDN4 antibody is possibly applicable to treatment or diagnosis of cancer (PTL 1 and NPL 1). Besides, it has been described that use of both an anti-CLDN4 antibody and an anti-epidermal growth factor receptor (EGFR) antibody has an antitumor effect in an animal model (NPL 2).

Cluster of differentiation 137 (CD137, another name 4-1BB) is a molecule belonging to the tumor necrosis factor receptor superfamily (TNFRSF), and has been reported to express on a cell surface of an immune cell such as a T cell, a B cell, a natural killer (NK) cell, a dendritic cell, an eosinophil, or a mast cell. In particular, CD137 on a T cell binds to a CD137 ligand on an antigen presenting cell and is known to be involved in activation and survival of the T cell as a costimulatory molecule (NPL 3). An anti-CD137 agonist antibody presents an antitumor effect through activation of an immune cell in a tumor microenvironment in an animal model (NPL 4). Urelumab, that is, an anti-CD137 agonist antibody, presented a therapeutic effect in a clinical trial, but is reported to cause an adverse reaction of liver disorder (NPL 5).

As an innovative method by which cancer cell-selective cytotoxic activity can be obtained at a low antibody concentration, a bispecific T cell recruiting antibody with various antibody formats has been reported. A bispecific T cell recruiting antibody is a bispecific antibody containing an antibody against a tumor-associated antigen (TAA) expressing on a cancer cell surface, and an antibody binding to a T cell, and is started to be examined for an effect of these antibodies in T cell-mediated immunotherapy (NPL 6). As an antibody binding to a T cell, an anti-CD3 antibody is often used, and bispecific T cell recruiting antibodies to various TAAs are now being studied and developed.

Furthermore, bispecific T cell recruiting antibodies against CD137 and TAA are being earnestly studied in recent years. Studies are being made on an anti-GPC3/anti-CD137 bispecific antibody, an anti-HER2/anti-CD137 bispecific antibody, an anti-PDL1/anti-CD137 bispecific antibody, an anti-FAP/anti-CD137 bispecific antibody and the like recognizing TAAs of glypican 3 (GPC3), human epidermal growth factor receptor type 2 (HER2), programmed cell-death ligand 1 (PD-L1), and fibroblast activation protein (FAP) (PTLs 2 and 3, and NPLs 7 to 9).

There is, until now, however, no report on an anti-CLDN4/anti-CD137 bispecific antibody.

### CITATION LIST

### Patent Literature

PTL 1: WO2008/114733
PTL 2: WO2015/156268
PTL 3: WO2016/177802

### Non Patent Literature

NPL 1: Cancer Science, 2009: 100 (9): pp. 1623-1630
NPL 2: Oncotarget, 2018: 9 (100): p. 37367-37378
NPL 3: Cancer Science, 2020: 111 (5): p. 1461-1467
NPL 4: Cancer Immunology Immunotherapy, 2012: 61 (5): p. 1721-1733
NPL 5: Clinical Cancer Research, 2017: 23 (8): p. 1929-1936
NPL 6: mAbs, 2017: 9 (2): p. 182-212
NPL 7: Clinical Cancer Research, 2019: 25 (19): p. 5878-5889
NPL 8: Clinical Cancer Research, 2020: 26 (15): p. 4154-4167
NPL 9: Journal for Immunotherapy of Cancer, 2020; 8 (2): e000238

### SUMMARY OF INVENTION

### Technical Problem

An object of the present invention is to provide an anti-CLDN4/anti-CD137 bispecific antibody usable in treatment of cancer.

### Solution to Problem

The present inventors searched for antibodies selectively bind to an antigen expressed in a peritoneal metastatic cancer cell to obtain 3D11 that is an anti-CLDN4 antibody (Examples 1 and 2). A bispecific T cell recruiting antibody using 3D11 and an anti-CD3 antibody was found to be unusable for treating a cancer patient (Reference Example). Therefore, 3D11 and an antibody KM3900, which is a known anti-CLDN4 antibody, were used to try to obtain the bispecific T cell recruiting antibody with the anti-CD137 antibody (Examples 3 and 4). The thus obtained anti-CLDN4/anti-CD137 bispecific antibody bound to CLDN4 and CD137 (Example 5), promoted production of interferon γ of a T cell in vitro, and exhibited cytotoxic activity to a cancer cell expressing CLDN4 on a cell surface thereof (Example 6). Besides, it was confirmed that the obtained anti-CLDN4/anti-CD137 bispecific antibody exerted antitumor activity in an in vivo mouse model (Example 7-1) and could be used safely in a cynomolgus monkey (Examples 7-2). It has been suggested that the anti-CLDN4/anti-CD137 bispecific antibody of the present invention is useful for the treatment of cancer.

Specifically, the present invention relates to the following [1] to [32].

[1] An anti-CLDN4/anti-CD137 bispecific antibody comprising a heavy chain variable region and a light chain variable region of an anti-CLDN4 antibody, and a heavy chain variable region and a light chain variable region of an anti-CD137 antibody, wherein the heavy chain variable region and the light chain variable region of the anti-CLDN4 antibody are either of the following (a) or (b):
(a) a heavy chain variable region containing a CDR1 consisting of an amino acid sequence at amino acid positions 31-35 of SEQ ID NO: 2, a CDR2 consisting of an amino acid sequence at amino acid positions 50-66 of SEQ ID NO: 2, and a CDR3 consisting of an amino acid sequence at amino acid positions 99-114 of SEQ ID NO: 2, and a light chain variable region containing a CDR1 consisting of an amino acid sequence at amino acid positions 24-35 of SEQ ID NO: 4, a CDR2 consisting of an amino acid sequence at amino acid positions 51-57 of SEQ ID NO: 4, and a CDR3 consisting of an amino acid sequence at amino acid positions 90-98 of SEQ ID NO: 4; or
(b) a heavy chain variable region containing a CDR1 consisting of an amino acid sequence at amino acid positions 31-35 of SEQ ID NO: 6, a CDR2 consisting of an amino acid sequence at amino acid positions 50-66 of SEQ ID NO: 6, and a CDR3 consisting of an amino acid sequence at amino acid positions 99-112 of SEQ ID NO: 6, and a light chain variable region containing a CDR1 consisting of an amino acid sequence at amino acid positions 24-35 of SEQ ID NO: 8, a CDR2 consisting of an amino acid sequence at amino acid positions 51-57 of SEQ ID NO: 8, and a CDR3 consisting of an amino acid sequence at amino acid positions 90-98 of SEQ ID NO: 8.

[2] The bispecific antibody according to [1], wherein the heavy chain variable region and the light chain variable region of the anti-CLDN4 antibody are either of the following (a) or (b):
(a) a heavy chain variable region consisting of an amino acid sequence at amino acid positions 1-125 of SEQ ID NO: 2 and a light chain variable region consisting of an amino acid sequence at amino acid positions 1-109 of SEQ ID NO: 4; or
(b) a heavy chain variable region consisting of an amino acid sequence at amino acid positions 1-123 of SEQ ID NO: 6 and a light chain variable region consisting of an amino acid sequence at amino acid positions 1-109 of SEQ ID NO: 8.

[3] The bispecific antibody according to any of [1] and [2], comprising an IgG antibody (anti-CLDN4 IgG antibody) consisting of a heavy chain containing the heavy chain variable region and a light chain containing the light chain variable region of the anti-CLDN4 antibody.

[4] The bispecific antibody according to [3], comprising LALA mutation (L234A and L235A, wherein a position of the mutation is an amino acid position in human Igyl constant region according to EU index) in a Fc region of the anti-CLDN4 IgG antibody.

[5] The anti-CLDN4/anti-CD137 bispecific antibody according to [3], comprising P331G mutation in a Fc region of the anti-CLDN4 IgG antibody (wherein a position of the mutation is an amino acid position in human Igyl constant region according to EU index).

[6] The bispecific antibody according to [3], comprising LALA mutation and P331G mutation in a Fc region of the anti-CLDN4 IgG antibody.

[7] The bispecific antibody according to any one of [1] to [6], wherein the heavy chain variable region and the light chain variable region of the anti-CD137 antibody are any of the following (a) to (d):
(a) a heavy chain variable region containing a CDR1 consisting of an amino acid sequence at amino acid positions 31-35 of SEQ ID NO: 10, a CDR2 consisting of an amino acid sequence at amino acid positions 50-66 of SEQ ID NO: 10, and a CDR3 consisting of an amino acid sequence at amino acid positions 99-107 of SEQ ID NO: 10, and a light chain variable region containing a CDR1 consisting of an amino acid sequence at amino acid positions 24-34 of SEQ ID NO: 12, a CDR2 consisting of an amino acid sequence at amino acid positions 50-56 of SEQ ID NO: 12, and a CDR3 consisting of an amino acid sequence at amino acid positions 89-98 of SEQ ID NO: 12;
(b) a heavy chain variable region containing a CDR1 consisting of an amino acid sequence at amino acid positions 31-35 of SEQ ID NO: 14, a CDR2 consisting of an amino acid sequence at amino acid positions 50-65 of SEQ ID NO: 14, and a CDR3 consisting of an amino acid sequence at amino acid positions 98-107 of SEQ ID NO: 14, and a light chain variable region containing a CDR1 consisting of an amino acid sequence at amino acid positions 23-35 of SEQ ID NO: 16, a CDR2 consisting of an amino acid sequence at amino acid positions 51-57 of SEQ ID NO: 16, and a CDR3 consisting of an amino acid sequence at amino acid positions 90-100 of SEQ ID NO: 16;
(c) a heavy chain variable region containing a CDR1 consisting of an amino acid sequence at amino acid positions 31-35 of SEQ ID NO: 18, a CDR2 consisting of an amino acid sequence at amino acid positions 50-65 of SEQ ID NO: 18, and a CDR3 consisting of an amino acid sequence at amino acid positions 98-107 of SEQ ID NO: 18, and a light chain variable region containing a CDR1 consisting of an amino acid sequence at amino acid positions 23-35 of SEQ ID NO: 20, a CDR2 consisting of an amino acid sequence at amino acid positions 51-57 of SEQ ID NO: 20, and a CDR3 consisting of an amino acid sequence at amino acid positions 90-100 of SEQ ID NO: 20; or
(d) a heavy chain variable region containing a CDR1 consisting of an amino acid sequence at amino acid positions 31-35 of SEQ ID NO: 22, a CDR2 consisting of an amino acid sequence at amino acid positions 50-65 of SEQ ID NO: 22, and a CDR3 consisting of an amino acid sequence at amino acid positions 98-110 of SEQ ID NO: 22, and a light chain variable region containing a CDR1 consisting of an amino acid sequence at amino acid positions 23-35 of SEQ ID NO: 24, a CDR2 consisting of an amino acid sequence at amino acid positions 51-57 of SEQ ID NO: 24, and a CDR3 consisting of an amino acid sequence at amino acid positions 90-100 of SEQ ID NO: 24.

[8] The bispecific antibody according to any of [1] to [7], wherein the heavy chain variable region and the light chain variable region of the anti-CD137 antibody are any of the following (a) to (i):
(a) a heavy chain variable region consisting of an amino acid sequence at amino acid positions 1-118 of SEQ ID NO: 10 and a light chain variable region consisting of an amino acid sequence at amino acid positions 1-109 of SEQ ID NO: 12;
(b) a heavy chain variable region consisting of an amino acid sequence at amino acid positions 1-118 of SEQ ID NO: 14 and a light chain variable region consisting of an amino acid sequence at amino acid positions 1-111 of SEQ ID NO: 16;
(c) a heavy chain variable region consisting of an amino acid sequence at amino acid positions 1-118 of SEQ ID NO: 18 and a light chain variable region consisting of an amino acid sequence at amino acid positions 1-111 of SEQ ID NO: 20;
(d) a heavy chain variable region consisting of an amino acid sequence at amino acid positions 1-121 of SEQ ID NO: 22 and a light chain variable region consisting of an amino acid sequence at amino acid positions 1-111 of SEQ ID NO: 24;
(e) a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26;
(f) a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28;
(g) a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30;
(h) a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32; or
(i) a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34.

[9] The bispecific antibody according to any of [7] and [8], comprising an anti-CD137 single chain variable fragment (anti-CD137 scFv) containing the heavy chain variable region and the light chain variable region of the anti-CD137 antibody.

[10] The bispecific antibody according to [9], wherein the anti-CD137 scFv is any of the following (a) to (e):
(a) an anti-CD137 scFv consisting of an amino acid sequence at amino acid positions 1-242 of SEQ ID NO: 26;
(b) an anti-CD137 scFv consisting of an amino acid sequence at amino acid positions 1-244 of SEQ ID NO: 28;
(c) an anti-CD137 scFv consisting of an amino acid sequence at amino acid positions 1-249 of SEQ ID NO: 30;
(d) an anti-CD137 scFv consisting of an amino acid sequence at amino acid positions 1-244 of SEQ ID NO: 32; or
(e) an anti-CD137 scFv consisting of an amino acid sequence at amino acid positions 1-247 of SEQ ID NO: 34.

[11] The bispecific antibody according to any of [9] and [10], comprising an anti-CLDN4 IgG antibody and the anti-CD137 scFv, wherein an amino terminal of the anti-CD137 scFv is linked to a heavy chain or light chain carboxy terminal of the anti-CLDN4 IgG antibody via a linker.

[12] An anti-CLDN4/anti-CD137 bispecific antibody described in any of the following (a) to (j):
(a) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26, wherein an amino terminal of the anti-CD137 scFv is linked to a heavy chain carboxy terminal of the anti-CLDN4 antibody via a linker;
(b) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28, wherein an amino terminal of the anti-CD137 scFv is linked to a heavy chain carboxy terminal of the anti-CLDN4 antibody via a linker;
(c) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30, wherein an amino terminal of the anti-CD137 scFv is linked to a heavy chain carboxy terminal of the anti-CLDN4 antibody via a linker;
(d) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32, wherein an amino terminal of the anti-CD137 scFv is linked to a heavy chain carboxy terminal of the anti-CLDN4 antibody via a linker;
(e) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34, wherein an amino terminal of the anti-CD137 scFv is linked to a heavy chain carboxy terminal of the anti-CLDN4 antibody via a linker;
(f) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26, wherein an amino terminal of the anti-CD137 scFv is linked to a heavy chain carboxy terminal of the anti-CLDN4 antibody via a linker;
(g) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28, wherein an amino terminal of the anti-CD137 scFv is linked to a heavy chain carboxy terminal of the anti-CLDN4 antibody via a linker;
(h) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30, wherein an amino terminal of the anti-CD137 scFv is linked to a heavy chain carboxy terminal of the anti-CLDN4 antibody via a linker;
(i) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32, wherein an amino terminal of the anti-CD137 scFv is linked to a heavy chain carboxy terminal of the anti-CLDN4 antibody via a linker; or
(j) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34, wherein an amino terminal of the anti-CD137 scFv is linked to a heavy chain carboxy terminal of the anti-CLDN4 antibody via a linker.

[13] The anti-CLDN4/anti-CD137 bispecific antibody according to any of and [12], wherein the linker is a GS linker.

[14] The anti-CLDN4/anti-CD137 bispecific antibody according to [13], wherein the GS linker is a linker consisting of an amino acid sequence of SEQ ID NO: 54.

[15] An anti-CLDN4/anti-CD137 bispecific antibody described in either of the following (a) or (b):
(a) a bispecific antibody comprising a polypeptide consisting of an amino acid sequence at amino acid positions 1-705 of SEQ ID NO: 36 containing a heavy chain of an anti-CLDN4 antibody and an anti-CD137 scFv, and a light chain of an anti-CLDN4 antibody consisting of an amino acid sequence at amino acid positions 1-215 of SEQ ID NO: 40; or
(b) a bispecific antibody comprising a polypeptide consisting of an amino acid sequence at amino acid positions 1-712 of SEQ ID NO: 38 containing a heavy chain of an anti-CLDN4 antibody and an anti-CD137 scFv, and a light chain of an anti-CLDN4 antibody consisting of an amino acid sequence at amino acid positions 1-215 of SEQ ID NO: 40.

[16] The anti-CLDN4/anti-CD137 bispecific antibody according to any of [1] to [15], which is post-translationally modified.

[17] The anti-CLDN4/anti-CD137 bispecific antibody according to [16], wherein the post-translational modification is pyroglutamylation at an N-terminal of a heavy chain variable region and/or lysine deletion at a heavy chain C-terminal.

[18] A polynucleotide having a nucleotide sequence encoding a heavy chain variable region or light chain variable region of an anti-CLDN4 antibody, which is selected from the group consisting of the following (a) to (d):
(a) a polynucleotide having a nucleotide sequence encoding a heavy chain variable region of an anti-CLDN4 antibody consisting of an amino acid sequence at amino acid positions 1-125 of SEQ ID NO: 2;
(b) a polynucleotide having a nucleotide sequence encoding a light chain variable region of an anti-CLDN4 antibody consisting of an amino acid sequence at amino acid positions 1-109 of SEQ ID NO: 4;
(c) a polynucleotide having a nucleotide sequence encoding a heavy chain variable region of an anti-CLDN4 antibody consisting of an amino acid sequence at amino acid positions 1-123 of SEQ ID NO: 6; or
(d) a polynucleotide having a nucleotide sequence encoding a light chain variable region of an anti-CLDN4 antibody consisting of an amino acid sequence at amino acid positions 1-109 of SEQ ID NO: 8.

[19] A polynucleotide having a nucleotide sequence encoding a heavy chain variable region or light chain variable region of an anti-CD137 antibody, which is selected from the group consisting of the following (a) to (r):
(a) a polynucleotide having a nucleotide sequence encoding a heavy chain variable region of an anti-CD137 antibody consisting of an amino acid sequence at amino acid positions 1-118 of SEQ ID NO: 10;
(b) a polynucleotide having a nucleotide sequence encoding a light chain variable region of an anti-CD137 antibody consisting of an amino acid sequence at amino acid positions 1-109 of SEQ ID NO: 12;
(c) a polynucleotide having a nucleotide sequence encoding a heavy chain variable region of an anti-CD137 antibody consisting of an amino acid sequence at amino acid positions 1-118 of SEQ ID NO: 14;
(d) a polynucleotide having a nucleotide sequence encoding a light chain variable region of an anti-CD137 antibody consisting of an amino acid sequence at amino acid positions 1-111 of SEQ ID NO: 16;
(e) a polynucleotide having a nucleotide sequence encoding a heavy chain variable region of an anti-CD137 antibody consisting of an amino acid sequence at amino acid positions 1-118 of SEQ ID NO: 18;
(f) a polynucleotide having a nucleotide sequence encoding a light chain variable region of an anti-CD137 antibody consisting of an amino acid sequence at amino acid positions 1-111 of SEQ ID NO: 20;
(g) a polynucleotide having a nucleotide sequence encoding a heavy chain variable region of an anti-CD137 antibody consisting of an amino acid sequence at amino acid positions 1-121 of SEQ ID NO: 22;
(h) a polynucleotide having a nucleotide sequence encoding a light chain variable region of an anti-CD137 antibody consisting of an amino acid sequence at amino acid positions 1-111 of SEQ ID NO: 24;
(i) a polynucleotide having a nucleotide sequence encoding a heavy chain variable region of an anti-CD137 antibody consisting of an amino acid sequence at amino acid positions 1-118 of SEQ ID NO: 26;
(j) a polynucleotide having a nucleotide sequence encoding a light chain variable region of an anti-CD137 antibody consisting of an amino acid sequence at amino acid positions 134-242 of SEQ ID NO: 26;
(k) a polynucleotide having a nucleotide sequence encoding a heavy chain variable region of an anti-CD137 antibody consisting of an amino acid sequence at amino acid positions 1-118 of SEQ ID NO: 28;
(1) a polynucleotide having a nucleotide sequence encoding a light chain variable region of an anti-CD137 antibody consisting of an amino acid sequence at amino acid positions 134-244 of SEQ ID NO: 28;
(m) a polynucleotide having a nucleotide sequence encoding a light chain variable region of an anti-CD137 antibody consisting of an amino acid sequence at amino acid positions 1-111 of SEQ ID NO: 30;
(n) a polynucleotide having a nucleotide sequence encoding a heavy chain variable region of an anti-CD137 antibody consisting of an amino acid sequence at amino acid positions 132-249 of SEQ ID NO: 30;
(o) a polynucleotide having a nucleotide sequence encoding a heavy chain variable region of an anti-CD137 antibody consisting of an amino acid sequence at amino acid positions 1-118 of SEQ ID NO: 32;
(p) a polynucleotide having a nucleotide sequence encoding a light chain variable region of an anti-CD137 antibody consisting of an amino acid sequence at amino acid positions 134-244 of SEQ ID NO: 32;
(q) a polynucleotide having a nucleotide sequence encoding a heavy chain variable region of an anti-CD137 antibody consisting of an amino acid sequence at amino acid positions 1-121 of SEQ ID NO: 34; or
(r) a polynucleotide having a nucleotide sequence encoding a light chain variable region of an anti-CD137 antibody consisting of an amino acid sequence at amino acid positions 137-247 of SEQ ID NO: 34.

[20] A polynucleotide having a nucleotide sequence encoding an anti-CD137 scFv, which is selected from the group consisting of the following (a) to (e):
(a) a polynucleotide having a nucleotide sequence encoding an anti-CD137 scFv consisting of an amino acid sequence at amino acid positions 1-242 of SEQ ID NO: 26;
(b) a polynucleotide having a nucleotide sequence encoding an anti-CD137 scFv consisting of an amino acid sequence at amino acid positions 1-244 of SEQ ID NO: 28;
(c) a polynucleotide having a nucleotide sequence encoding an anti-CD137 scFv consisting of an amino acid sequence at amino acid positions 1-249 of SEQ ID NO: 30;
(d) a polynucleotide having a nucleotide sequence encoding of an anti-CD137 scFv consisting of an amino acid sequence at amino acid positions 1-244 of SEQ ID NO: 32; or
(e) a polynucleotide having a nucleotide sequence encoding an anti-CD137 scFv consisting of an amino acid sequence at amino acid positions 1-247 of SEQ ID NO: 34.

[21] A polynucleotide for use in production of the anti-CLDN4/anti-CD137 bispecific antibody according to [15], which is selected from the group consisting of the following (a) to (e):
(a) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv;
(b) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv;
(c) a polynucleotide having a nucleotide sequence encoding a light chain of an anti-CLDN4 antibody consisting of an amino acid sequence at amino acid positions 1-215 of SEQ ID NO: 40;
(d) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40; or
(e) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40.

[22] An expression vector, comprising the polynucleotide according to any of to [21].

[23] A host cell transformed with the expression vector according to [22].

[24] A host cell comprising a polynucleotide selected from the group consisting of the following (a) to (dd):
(a) a polynucleotide having a nucleotide sequence encoding a heavy chain variable region of an anti-CLDN4 antibody consisting of an amino acid sequence at amino acid positions 1-125 of SEQ ID NO: 2;
(b) a polynucleotide having a nucleotide sequence encoding a light chain variable region of an anti-CLDN4 antibody consisting of an amino acid sequence at amino acid positions 1-109 of SEQ ID NO: 4;
(c) a polynucleotide having a nucleotide sequence encoding a heavy chain variable region of an anti-CLDN4 antibody consisting of an amino acid sequence at amino acid positions 1-123 of SEQ ID NO: 6;
(d) a polynucleotide having a nucleotide sequence encoding a light chain variable region of an anti-CLDN4 antibody consisting of an amino acid sequence at amino acid positions 1-109 of SEQ ID NO: 8;
(e) a polynucleotide having a nucleotide sequence encoding a heavy chain variable region of an anti-CD137 antibody consisting of an amino acid sequence at amino acid positions 1-118 of SEQ ID NO: 10;
(f) a polynucleotide having a nucleotide sequence encoding a light chain variable region of an anti-CD137 antibody consisting of an amino acid sequence at amino acid positions 1-109 of SEQ ID NO: 12;
(g) a polynucleotide having a nucleotide sequence encoding a heavy chain variable region of an anti-CD137 antibody consisting of an amino acid sequence at amino acid positions 1-118 of SEQ ID NO: 14;
(h) a polynucleotide having a nucleotide sequence encoding a light chain variable region of an anti-CD137 antibody consisting of an amino acid sequence at amino acid positions 1-111 of SEQ ID NO: 16;
(i) a polynucleotide having a nucleotide sequence encoding a heavy chain variable region of an anti-CD137 antibody consisting of an amino acid sequence at amino acid positions 1-118 of SEQ ID NO: 18;
(j) a polynucleotide having a nucleotide sequence encoding a light chain variable region of an anti-CD137 antibody consisting of an amino acid sequence at amino acid positions 1-111 of SEQ ID NO: 20;
(k) a polynucleotide having a nucleotide sequence encoding a heavy chain variable region of an anti-CD137 antibody consisting of an amino acid sequence at amino acid positions 1-121 of SEQ ID NO: 22;
(1) a polynucleotide having a nucleotide sequence encoding a light chain variable region of an anti-CD137 antibody consisting of an amino acid sequence at amino acid positions 1-111 of SEQ ID NO: 24;
(m) a polynucleotide having a nucleotide sequence encoding a heavy chain variable region of an anti-CD137 antibody consisting of an amino acid sequence at amino acid positions 1-118 of SEQ ID NO: 26;
(n) a polynucleotide having a nucleotide sequence encoding a light chain variable region of an anti-CD137 antibody consisting of an amino acid sequence at amino acid positions 134-242 of SEQ ID NO: 26;
(o) a polynucleotide having a nucleotide sequence encoding a heavy chain variable region of an anti-CD137 antibody consisting of an amino acid sequence at amino acid positions 1-118 of SEQ ID NO: 28;
(p) a polynucleotide having a nucleotide sequence encoding a light chain variable region of an anti-CD137 antibody consisting of an amino acid sequence at amino acid positions 134-244 of SEQ ID NO: 28;
(q) a polynucleotide having a nucleotide sequence encoding a light chain variable region of an anti-CD137 antibody consisting of an amino acid sequence at amino acid positions 1-111 of SEQ ID NO: 30;
(r) a polynucleotide having a nucleotide sequence encoding a heavy chain variable region of an anti-CD137 antibody consisting of an amino acid sequence at amino acid positions 132-249 of SEQ ID NO: 30;
(s) a polynucleotide having a nucleotide sequence encoding a heavy chain variable region of an anti-CD137 antibody consisting of an amino acid sequence at amino acid positions 1-118 of SEQ ID NO: 32;
(t) a polynucleotide having a nucleotide sequence encoding a light chain variable region of an anti-CD137 antibody consisting of an amino acid sequence at amino acid positions 134-244 of SEQ ID NO: 32;
(u) a polynucleotide having a nucleotide sequence encoding a heavy chain variable region of an anti-CD137 antibody consisting of an amino acid sequence at amino acid positions 1-121 of SEQ ID NO: 34;
(v) a polynucleotide having a nucleotide sequence encoding a light chain variable region of an anti-CD137 antibody consisting of an amino acid sequence at amino acid positions 137-247 of SEQ ID NO: 34;
(w) a polynucleotide having a nucleotide sequence encoding an anti-CD137 scFv consisting of an amino acid sequence at amino acid positions 1-242 of SEQ ID NO: 26;
(x) a polynucleotide having a nucleotide sequence encoding an anti-CD137 scFv consisting of an amino acid sequence at amino acid positions 1-244 of SEQ ID NO: 28;
(y) a polynucleotide having a nucleotide sequence encoding an anti-CD137 scFv consisting of an amino acid sequence at amino acid positions 1-249 of SEQ ID NO: 30;
(z) a polynucleotide having a nucleotide sequence encoding an anti-CD137 scFv consisting of an amino acid sequence at amino acid positions 1-244 of SEQ ID NO: 32;
(aa) a polynucleotide having a nucleotide sequence encoding an anti-CD137 scFv consisting of an amino acid sequence at amino acid positions 1-247 of SEQ ID NO: 34;
(bb) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv;
(cc) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv; or (dd) a polynucleotide having a nucleotide sequence encoding a light chain of an anti-CLDN4 antibody consisting of an amino acid sequence at amino acid positions 1-215 of SEQ ID NO: 40.

[25] A host cell comprising a polynucleotide of either of the following (a) or (b):
(a) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40; or
(b) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40.

[26] A method for producing an anti-CLDN4/anti-CD137 bispecific antibody, comprising a step of culturing the host cell according to any of to [25].

[27] A pharmaceutical composition comprising the anti-CLDN4/anti-CD137 bispecific antibody according to any of [1] to [17], and a pharmaceutically acceptable excipient.

[28] The anti-CLDN4/anti-CD137 bispecific antibody according to any of [1] to for use in treatment of cancer.

[29] The pharmaceutical composition according to for use in treatment of cancer.

[30] A method for treating cancer, comprising a step of administering a therapeutically effective amount of the anti-CLDN4/anti-CD137 bispecific antibody according to any of [1] to to a subject.

[31] Use of the anti-CLDN4/anti-CD137 bispecific antibody according to any of [1] to in production of a pharmaceutical composition for use in treatment of cancer.

### Advantageous Effects of Invention

An anti-CLDN4/anti-CD137 bispecific antibody of the present invention binds both to CLDN4 that is a cancer antigen and CD137 expressing on a cell surface of an immune cell such as a T cell to activate immune cells present around a cancer cell, and thus enhances killing activity on the cancer cell. The anti-CLDN4/anti-CD137 bispecific antibody of the present invention or a pharmaceutical composition containing the antibody can be used for treatment of various cancers.

### BRIEF DESCRIPTION OF DRAWINGS

FIG. 1-1 illustrates binding activity to CD137 of test antibodies measured and evaluated by ELISA. The ordinate of the drawing indicates a value obtained by subtracting an absorbance at 570 nm from an absorbance at 450 nm, and the abscissa indicates a concentration of the test antibody. Each symbol indicates an average of the absorbance obtained in adding each of test antibodies, and an error bar indicates the standard deviation.

FIG. 1-2 illustrates binding activity to CD137 of test antibodies measured and evaluated by ELISA. The ordinate of the drawings indicates a value obtained by subtracting an absorbance at 570 nm from an absorbance at 450 nm, and the abscissa indicates a concentration of the test antibody. Each of symbols indicates an average of the absorbance obtained in adding each of test antibodies.

FIG. 1-3 illustrates binding activity to CD137 of test antibodies measured and evaluated by ELISA. The ordinate of the drawings indicates a value obtained by subtracting an absorbance at 570 nm from an absorbance at 450 nm, and the abscissa indicates a concentration of the test antibody. Each of symbols indicates an average of the absorbance obtained in adding each of test antibodies.

FIG. 1-4 illustrates binding activity to CD137 of test antibodies measured and evaluated by ELISA. The ordinate of the drawings indicates a value obtained by subtracting an absorbance at 570 nm from an absorbance at 450 nm, and the abscissa indicates a concentration of the test antibody. Each of symbols indicates an average of the absorbance obtained in adding each of test antibodies.

FIG. 2-1 illustrates CD137 agonistic activity of test antibodies obtained in a coculture test of CLDN4 expressing cancer cells (NCI-H322 cell) and 4-1BB Effector Cells. The ordinate of the drawings indicates a relative amount of luminescence, and the abscissa indicates a concentration of the test antibody. Each of symbols indicates an average of the relative amount of luminescence obtained in adding each of test antibodies, and an error bar indicates the standard deviation.

FIG. 2-2 illustrates CD137 agonistic activity of test antibodies obtained in a coculture test of CLDN4 expressing cancer cells (NCI-H322 cell) and 4-1BB Effector Cells. The ordinate of the drawing indicates a relative amount of luminescence, and the abscissa indicates a concentration of the test antibody. Each of symbols indicates an average of the relative amount of luminescence obtained in adding each of test antibodies.

FIG. 2-3 illustrates CD137 agonistic activity of test antibodies obtained in a coculture test of CLDN4 expressing cancer cells (NCI-H322 cell) and 4-1BB Effector Cells. The ordinate of the drawings indicates a relative amount of luminescence, and the abscissa indicates a concentration of the test antibody. Each of symbols indicates an average of the relative amount of luminescence obtained in adding each of test antibodies, and an error bar indicates the standard deviation.

FIG. 2-4 illustrates CD137 agonistic activity of test antibodies obtained in a monoculture test of 4-1BB Effector Cells. The ordinate of the drawings indicates a relative amount of luminescence, and the abscissa indicates a concentration of the test antibody. Each of symbols indicates an average of the relative amount of luminescence obtained in adding each of test antibodies, and an error bar indicates the standard deviation.

FIG. 2-5 illustrates CD137 agonistic activity of test antibodies obtained in a monoculture test of 4-1BB Effector Cells. The ordinate of the drawing indicates a relative amount of luminescence, and the abscissa indicates a concentration of the test antibody. Each of symbols indicates an average of the relative amount of luminescence obtained in adding each of test antibodies.

FIG. 2-6 illustrates CD137 agonistic activity of test antibodies obtained in a monoculture test of 4-1BB Effector Cells. The ordinate of the drawings indicates a relative amount of luminescence, and the abscissa indicates a concentration of the test antibody. Each of symbols indicates an average of the relative amount of luminescence obtained in adding each of test antibodies, and an error bar indicates the standard deviation.

FIG. 3-1 illustrates interferon-γ production promoting function obtained in adding each test antibody to a coculture system of 60As6-Luc/GFP cells and expanded pan T cells. The ordinate indicates a production amount (pg/mL) of interferon γ obtained 6 days after the antibody addition, and the abscissa indicates a concentration of the antibody. Each of symbols indicates an average of the production amount of interferon γ at each concentration of the antibody.

FIG. 3-2 illustrates interferon-γ production promoting function obtained in adding each test antibody to a coculture system of 60As6-Luc/GFP cells and expanded pan T cells. The ordinate of the drawing indicates a production amount (pg/mL) of interferon γ obtained 4 days after the antibody addition, and the abscissa indicates a concentration of the antibody. Each of symbols indicates an average of the production amount of interferon γ at each concentration of the antibody, and an error bar indicates the standard deviation.

FIG. 3-3 illustrates a production amount of interferon γ obtained in adding each test antibody to a monoculture system of expanded pan T cells. The ordinate of the drawing indicates a production amount (pg/mL) of interferon γ obtained 6 days after the antibody addition, and the abscissa indicates a concentration of the antibody. Each of symbols indicates an average of the production amount of interferon γ at each concentration of the antibody.

FIG. 3-4 illustrates a production amount of interferon γ obtained in adding each test antibody to a monoculture system of expanded pan T cells. The ordinate of the drawing indicates a production amount (pg/mL) of interferon γ obtained 5 days after the antibody addition, and the abscissa indicates a concentration of the antibody. Each of symbols indicates an average of the production amount of interferon γ at each concentration of the antibody, and an error bar indicates the standard deviation.

FIG. 4 illustrates cancer cytotoxic activity (cancer cell growth inhibitory activity) by each test antibody in a coculture system of 60As6-Luc/GFP cells and expanded pan T cells. The ordinate of the drawing indicates a cell growth rate obtained assuming that, in a fluorescence area increased from 0 hours to 168 hours after the start of measurement, a well where the test antibody was not added corresponds to 100%. The abscissa indicates a concentration of the antibody. Each of symbols indicates an average of the cancer cell growth rate in each antibody.

FIG. 5 illustrates cancer cell cytotoxic activity obtained in adding each test antibody to a coculture system of 60As6-Luc/GFP cells and expanded pan T cells. The ordinate of the drawing indicates a cancer cell death rate (%) obtained assuming that, in measured values of chemiluminescence of the 60As6-Luc/GFP cells measured 6 days after the antibody addition, an average obtained without adding the antibody corresponds to a death rate of 0%, and an average obtained with Triton-X100 added corresponds to a death rate of 100%. The abscissa indicates a concentration of the antibody. Each of symbols indicates an average of the cancer cell death rate at each concentration of the antibody, and an error bar indicates the standard deviation.

FIG. 6 illustrates a tumor volume change in a PBMC-transferred mouse subcutaneously bearing cancer of an NCI-H322 cell. The abscissa of the drawing indicates days after the first administration, the ordinate indicates an average of the tumor volume, and an error bar indicates the standard deviation.

### DESCRIPTION OF EMBODIMENTS

The present invention will now be described in detail.

### Definitions

Terms used herein are used to have meanings generally used in this technical field by those skilled in the art unless especially defined in the following.

An antibody (or immunoglobulin) is a glycoprotein whose basic structure is a four-chain structure of a bilaterally symmetrical Y-shape including two heavy chains having a single sequence and two light chains having a single sequence. There are five classes of antibody, that is, IgG, IgM, IgA, IgD and IgE. A basic structure of an antibody molecule is common among these classes, and two heavy chains having a molecular weight of 50,000 to 70,000 and two light chains having a molecular weight of 20,000 to 30,000 are bound via a disulfide bond and a noncovalent bond to form an antibody molecule having a Y-shaped four chain structure with a molecular weight of 150,000 to 190,000. The heavy chain is a polypeptide chain usually containing about 440 amino acids, and has a structure peculiar to each class, and is designated respectively as Igγ, Igμ, Igα, Igδ, and Igε correspondingly to IgG, IgM, IgA, IgD, or IgE. IgG has subclasses of IgG1, IgG2, IgG3 and IgG4, and heavy chains respectively corresponding to these subclasses are designated as Igyl, Igγ2, Igγ3, and Igγ4. The light chain is a polypeptide chain usually containing about 220 amino acids, and it is known to have two types, λ type and κ type, which are respectively designated as Igλ and Igκ. Each of the two types of the light chains can be paired with any type of the heavy chain.

In an antibody molecule, the heavy chain has four intrachain disulfide bonds (five in Igμ and Igε), the light chain has two intrachain disulfide bonds, and thus, one loop is formed every 100 to 110 amino acid residues. The three-dimensional structures of these are similar among the loops, and are designated as a structural unit or a domain. In both the heavy chain and the light chain, a domain positioned at the N-terminal is designated as a variable region, and has an amino acid sequence varied even among antibodies produced from the same class (or subclass) of the same species of animal, and is known to be involved in antibody-antigen bond specific binding. A domain disposed on the downstream side from the variable region on the C-terminal side has an amino acid sequence substantially constant in each class or subclass, and is designated as a constant region. The heavy chain has, in a direction from the N-terminal to the C-terminal, a heavy chain variable region (VH) and a heavy chain constant region (CH). The CH is further divided, from the N-terminal side, into three domains of a CH1 domain, a CH2 domain and a CH3 domain. The light chain has, in a direction from the N-terminal to the C-terminal, a light chain variable region (VL) and a light chain constant region (CL).

An amino acid sequence of three complementarity determining regions (CDRs) present in the VH and VL is largely varied, and contributes to variability of the variable regions. Each CDR is a region consisting of 5 to 10 amino acid residues, which is present in the order of CDR1, CDR2 and CDR3 in the N-terminal of each of the heavy chain and the light chain, and comes into contact with an antigen to form an antibody binding site. It is known that the CDRs of the heavy chain make a larger contribution to antigen binding than the CDRs of the light chain, and that the CDR3 makes a largest contribution among the CDR1 to CDR3. On the other hand, a portion excluding the CDRs of the variable region is designated as a framework region (FR), consists of FR1 to FR4, and is comparatively little varied in the amino acid sequence.

When an antibody is treated with papain, that is, a protease, three antibody fragments are obtained. Two fragments disposed on the N-terminal side are designated as a Fab (antigen binding fragment, Fragment, antigen binding) region. The Fab region refers to a region consisting of the VH of the heavy chain, a part of the CH1 domain and a hinge region, and the light chain (VL and CL), and binds to an antigen at the VH and VL (antigen binding site) included in the Fab region. Besides, a fragment disposed on the C-terminal side is designated as a Fc (crystallizable fragment, Fragment, crystallizable) region.

Herein, an “IgG antibody” refers to an antibody having a Y-shaped structure including two Fab regions and a Fc region. In one embodiment, the two Fab regions of the IgG antibody contain the same Fab region sequences. In one embodiment, the two Fab regions of the IgG antibody contain different Fab region sequences.

Herein, an “antigen binding fragment” and a “fragment antibody” can be interchangeably used, and refer to a molecule that contains at least one polypeptide chain containing a heavy chain variable region and a light chain variable region of an immunoglobulin molecule, and having antigen binding activity. Representative examples of the antigen binding fragment include a single chain variable fragment (scFv), a Fab fragment, a Fab′ fragment, and a F(ab′)₂ fragment. Herein, a “scFv” refers to a monovalent antigen binding fragment containing a VH and a VL linked via a linker. A Fab fragment is a monovalent antigen binding fragment containing a fragment consisting of a light chain, a VH and a CH1 domain of a heavy chain, and a part of a hinge region. A Fab′ fragment is a monovalent antigen binding fragment containing a fragment consisting of a light chain, a VH and a CH1 domain of a heavy chain, and a part of a hinge region, and this part of the hinge region contains a cysteine residue contained in the disulfide bond between the heavy chains. A F(ab′)₂ fragment is a bivalent antigen binding fragment containing Fab′ fragments bounded via a disulfide bond. The term “monovalent” means that one antigen binding site is contained, and the term “bivalent” means that two antigen binding sites are contained.

Herein, a “bispecific antibody” refers to an antibody molecule containing two antibodies or antigen binding fragments specifically binding to different antigens, and having binding activity to the respective antigens. As bispecific antibodies, bispecific antibodies having various structures are known to those skilled in the art (NPL 6). The term “antibody” used herein encompasses full length antibodies (various immunoglobulins, especially an IgG antibody), an antigen binding fragment, and bispecific antibodies having any structure unless particularly limited contextually.

Herein, an IgG type anti-CLDN4 antibody is designated as an “anti-CLDN4 IgG antibody.”

Herein, a “human antibody” refers to an antibody having a human immunoglobulin amino acid sequence. Herein, a “humanized antibody” refers to an antibody in which a part of, most of, or all of amino acid residues excluding the CDRs are substituted with amino acid residues derived from a human immunoglobulin molecule. A humanization method is not especially limited, and for example, U.S. Pat. Nos. 5,225,539, 6,180,370 and the like can be referred to for producing a humanized antibody.

An amino acid residue number of an antibody used herein can be prescribed by specifying the Kabat numbering or EU index (Kabat et al., Sequences of Proteins of Immunological Interest, 5th Ed., 1991, NIH Publication No. 91-3242) in accordance with the numbering system.

Herein, the term “link” or “linked” means that a plurality of components (such as a Fab region and a Fc region) are connected directly or via an intermediary (such as a peptide linker). Herein, the term “peptide linker” means at least one amino acid sequence that can be introduced by a genetic engineering method used for linking a plurality of components. The length of a peptide linker used in the present invention is not especially limited, and can be appropriately selected by those skilled in the art depending on purpose.

Herein, the term “identity” means a value of identity obtained using EMBOSS Needle (Nucleic Acids Res., 2015, Vol. 43, p. W580-W584) with parameters prepared as default. The parameters are as follows:
Gap Open Penalty=10
Gap Extend Penalty=0.5
Matrix=EBLOSUM62
End Gap Penalty=false

### <Anti-CLDN4/anti-CD137 Bispecific Antibody of Invention>

The present invention provides a bispecific antibody binding to CLDN4 and CD137 (also referred to as the “anti-CLDN4/anti-CD137 bispecific antibody”). The anti-CLDN4/anti-CD137 bispecific antibody of the present invention contains a heavy chain variable region and a light chain variable region of an anti-CLDN4 antibody, and a heavy chain variable region and a light chain variable region of an anti-CD137 antibody. The anti-CLDN4/anti-CD137 bispecific antibody of the present invention contains a bispecific antibody containing an anti-CLDN4 IgG antibody and a scFv of an anti-CD137 antibody (also referred to as the “anti-CD137 scFv”). In one embodiment, the anti-CLDN4/anti-CD137 bispecific antibody of the present invention is a bispecific antibody containing the anti-CLDN4 IgG antibody and the anti-CD137 scFv.

Herein, the “anti-CLDN4 antibody” is an antibody capable of binding to human CLDN4, and the “anti-CD137 antibody” is an antibody capable of binding to human CD137. It can be checked by employing a known binding activity measurement method whether or not an antibody binds to human CLDN4 or human CD137. Examples of a method for measuring binding activity include a method such as enzyme-linked immunosorbent assay (ELISA) or flow cytometry. ELISA or flow cytometry can be carried out by a method usually employed by those skilled in the art, and a method described in, for example, Example 3 or 5 can be employed.

The anti-CLDN4/anti-CD137 bispecific antibody of the present invention may have any structure as long as it binds to CLDN4 and CD137, and examples thereof include a bispecific antibody having a structure described in NPL 6. In one embodiment, the anti-CLDN4/anti-CD137 bispecific antibody of the present invention may be a conjugate in which a Fab region of an anti-CLDN4 antibody and a Fab region of an anti-CD137 antibody are linked to each other, a conjugate of an anti-CLDN4 IgG antibody and an IgG type anti-CD137 antibody (also referred to as the “anti-CD137 IgG antibody”), a conjugate of an anti-CLDN4 IgG antibody and an antigen binding fragment of an anti-CD137 antibody, a conjugate of an antigen binding fragment of an anti-CLDN4 antibody and an anti-CD137 IgG antibody, or a conjugate of an antigen binding fragment of an anti-CLDN4 antibody and an antigen binding fragment of an anti-CD137 antibody.

In one embodiment, the anti-CLDN4/anti-CD137 bispecific antibody of the present invention contains a full length anti-CLDN4 antibody and a full length anti-CD137 antibody. In one embodiment, the anti-CLDN4/anti-CD137 bispecific antibody of the present invention contains an anti-CLDN4 IgG antibody and an anti-CD137 IgG antibody. Two antibodies may be linked to each other via a linker. In one embodiment, the anti-CLDN4/anti-CD137 bispecific antibody of the present invention contains a full length anti-CLDN4 antibody and an antigen binding fragment of an anti-CD137 antibody. A linkage site of the two antibodies is not especially limited, and they are linked directly or via a linker. In one embodiment, the anti-CLDN4/anti-CD137 bispecific antibody of the present invention contains an anti-CLDN4 IgG antibody and a scFv of an anti-CD137 antibody (herein referred to as the “anti-CD137 scFv”). In one embodiment, the anti-CLDN4/anti-CD137 bispecific antibody of the present invention contains an anti-CLDN4 IgG antibody and an anti-CD137 scFv, in which the amino terminal of the anti-CD137 scFv is linked to the heavy chain carboxy terminal of the anti-CLDN4 antibody. In one embodiment, the anti-CLDN4/anti-CD137 bispecific antibody of the present invention contains an anti-CLDN4 IgG antibody and an anti-CD137 scFv, in which the amino terminal of the anti-CD137 scFv is linked to the light chain carboxy terminal of the anti-CLDN4 antibody. In one embodiment, the anti-CLDN4/anti-CD137 bispecific antibody of the present invention contains an anti-CLDN4 IgG antibody and an anti-CD137 scFv, in which the carboxy terminal of the anti-CD137 scFv is linked to the amino terminal of a heavy chain of the anti-CLDN4 antibody. In one embodiment, the anti-CLDN4/anti-CD137 bispecific antibody of the present invention contains an anti-CLDN4 IgG antibody and an anti-CD137 scFv, in which the carboxy terminal of the anti-CD137 scFv is linked to the amino terminal of a light chain of the anti-CLDN4 antibody. In one embodiment, the anti-CLDN4/anti-CD137 bispecific antibody of the present invention contains an antigen binding fragment of an anti-CLDN4 antibody and an antigen binding fragment of anti-CD137. In one embodiment, the anti-CLDN4/anti-CD137 bispecific antibody of the present invention contains a scFv of an anti-CLDN4 antibody and a scFv of anti-CD137. In one embodiment, the anti-CLDN4/anti-CD137 bispecific antibody of the present invention contains an anti-CD137 scFv.

In the anti-CLDN4/anti-CD137 bispecific antibody of the present invention, the heavy chain variable regions and the light chain variable regions of the anti-CLDN4 antibody and the anti-CD137 antibody may be those derived from a human antibody, or derived from a humanized antibody, or a combination of these. In one embodiment, the anti-CLDN4/anti-CD137 bispecific antibody of the present invention is an antibody containing a human antibody, a humanized antibody, or a combination of these.

The anti-CLDN4/anti-CD137 bispecific antibody of the present invention is an anti-CLDN4/anti-CD137 bispecific antibody containing a heavy chain variable region and a light chain variable region of an anti-CLDN4 antibody, and a heavy chain variable region and a light chain variable region of an anti-CD137 antibody, wherein the heavy chain variable region and the light chain variable region of the anti-CLDN4 antibody are either of the following (a) or (b):
(a) a heavy chain variable region containing a CDR1 consisting of an amino acid sequence at amino acid positions 31-35 of SEQ ID NO: 2, a CDR2 consisting of an amino acid sequence at amino acid positions 50-66 of SEQ ID NO: 2, and a CDR3 consisting of an amino acid sequence at amino acid positions 99-114 of SEQ ID NO: 2, and a light chain variable region containing a CDR1 consisting of an amino acid sequence at amino acid positions 24-35 of SEQ ID NO: 4, a CDR2 consisting of an amino acid sequence at amino acid positions 51-57 of SEQ ID NO: 4, and a CDR3 consisting of an amino acid sequence at amino acid positions 90-98 of SEQ ID NO: 4; or
(b) a heavy chain variable region containing a CDR1 consisting of an amino acid sequence at amino acid positions 31-35 of SEQ ID NO: 6, a CDR2 consisting of an amino acid sequence at amino acid positions 50-66 of SEQ ID NO: 6, and a CDR3 consisting of an amino acid sequence at amino acid positions 99-112 of SEQ ID NO: 6, and a light chain variable region containing a CDR1 consisting of an amino acid sequence at amino acid positions 24-35 of SEQ ID NO: 8, a CDR2 consisting of an amino acid sequence at amino acid positions 51-57 of SEQ ID NO: 8, and a CDR3 consisting of an amino acid sequence at amino acid positions 90-98 of SEQ ID NO: 8.

In one embodiment, the anti-CLDN4/anti-CD137 bispecific antibody of the present invention contains a heavy chain variable region and a light chain variable region of an anti-CLDN4 antibody described in the following (a) or (b):
(a) a heavy chain variable region consisting of an amino acid sequence at amino acid positions 1-125 of SEQ ID NO: 2 and a light chain variable region consisting of an amino acid sequence at amino acid positions 1-109 of SEQ ID NO: 4; or
(b) a heavy chain variable region consisting of an amino acid sequence at amino acid positions 1-123 of SEQ ID NO: 6 and a light chain variable region consisting of an amino acid sequence at amino acid positions 1-109 of SEQ ID NO: 8.

The anti-CLDN4/anti-CD137 bispecific antibody of the present invention may contain, as an anti-CLDN4 antibody, a full length antibody such as an IgG antibody, or may contain an antigen binding fragment such as a Fab fragment, a Fab′ fragment, a F(ab′)₂ fragment, or a scFv.

As the heavy chain constant region contained in the anti-CLDN4 antibody contained in the anti-CLDN4/anti-CD137 bispecific antibody of the present invention, any of the constant regions Igγ, Igμ, Igα, Igδ and Igε can be selected. Igγ can be selected from, for example, Igγ1, Igγ2, Igγ3 and Igγ4. As the light chain constant region contained in the anti-CLDN4 antibody contained in the anti-CLDN4/anti-CD137 bispecific antibody of the present invention, either of the constant regions Igλ and Igκ can be selected. When the anti-CLDN4 antibody contained in the anti-CLDN4/anti-CD137 bispecific antibody of the present invention is an IgG antibody, the heavy chain and the light chain of the anti-CLDN4 antibody are respectively human Igγ1 and Igκ in one embodiment. In one embodiment, the anti-CLDN4/anti-CD137 bispecific antibody of the present invention contains a full length anti-CLDN4 antibody. In one embodiment, the anti-CLDN4 antibody contained in the anti-CLDN4/anti-CD137 bispecific antibody of the present invention is an IgG antibody containing a heavy chain variable region and a light chain variable region of an anti-CLDN4 antibody (anti-CLDN4 IgG antibody).

When the anti-CLDN4/anti-CD137 bispecific antibody of the present invention contains a Fc region, the Fc region of the bispecific antibody may contain mutation that deteriorates antibody-dependent cellular cytotoxicity (ADCC) or complement-dependent cytotoxicity (CDC). L234A is replacement of leucine with alanine at amino acid position 234 in human Igγ1 constant region. L235A is replacement of leucine with alanine at amino acid position 235 in human Igγ1 constant region. The amino acid mutation L234A and L235A of human Igγ1 constant region is designated as “LALA mutation.” Here, the positions 234 and 235 are amino acid positions in human Igγ1 constant region according to EU index. This mutation is known to deteriorate ADCC and CDC of an antibody (Mol. Immunol., 1992, Vol. 29, p. 633-639; and J. Immunol., 2000, Vol. 164, p. 4178-4184). P331G or P331S is replacement of proline with glycine or serine at amino acid position 331 in human Igγ1 constant region. Here, the position 331 is an amino acid position in human Igγ1 constant region according to EU index. This mutation is known to deteriorate CDC of an antibody (J. Immunol., 2000, Vol. 164 (8), p. 4178-4184).

In one embodiment, the anti-CLDN4 IgG antibody contained in the anti-CLDN4/anti-CD137 bispecific antibody of the present invention contains a Fc region containing amino acid mutation L234A and L235A (LALA mutation). In one embodiment, the anti-CLDN4 IgG antibody contains a Fc region containing either P331G or P331S mutation. In one embodiment, the anti-CLDN4 IgG antibody contains a Fc region containing mutations of LALA mutation and either P331G or P331S mutation.

The present inventors created a novel anti-CD137 antibody or its binding fragment (scFv) in the process of creating the anti-CLDN4/anti-CD137 bispecific antibody of the present invention. The present inventors identified an anti-CLDN4/anti-CD137 bispecific antibody that does not transduce a CD137 signal when binding only to CD137, but transduces the CD137 signal only when binding simultaneously to CD137 and CLDN4. The antibody does not exhibit agonistic activity when not binding to CLDN4. On the other hand, binding of the anti-CLDN4/anti-CD137 bispecific antibody to CLDN4 and CD137 leads to CD137 signal transduction and activation of a T cell. This activity is expected to alleviate hepatotoxicity or the like observed in a clinical trial of Urelumab, and therefore is expected to be a preferable profile as an anti-CD137 antibody used in the bispecific antibody.

In one embodiment, the anti-CLDN4/anti-CD137 bispecific antibody of the present invention contains a heavy chain variable region and a light chain variable region of an anti-CD137 antibody described in any of the following (a) to (d):
(a) a heavy chain variable region containing a CDR1 consisting of an amino acid sequence at amino acid positions 31-35 of SEQ ID NO: 10, a CDR2 consisting of an amino acid sequence at amino acid positions 50-66 of SEQ ID NO: 10, and a CDR3 consisting of an amino acid sequence at amino acid positions 99-107 of SEQ ID NO: 10, and a light chain variable region containing a CDR1 consisting of an amino acid sequence at amino acid positions 24-34 of SEQ ID NO: 12, a CDR2 consisting of an amino acid sequence at amino acid positions 50-56 of SEQ ID NO: 12, and a CDR3 consisting of an amino acid sequence at amino acid positions 89-98 of SEQ ID NO: 12;
(b) a heavy chain variable region containing a CDR1 consisting of an amino acid sequence at amino acid positions 31-35 of SEQ ID NO: 14, a CDR2 consisting of an amino acid sequence at amino acid positions 50-65 of SEQ ID NO: 14, and a CDR3 consisting of an amino acid sequence at amino acid positions 98-107 of SEQ ID NO: 14, and a light chain variable region containing a CDR1 consisting of an amino acid sequence at amino acid positions 23-35 of SEQ ID NO: 16, a CDR2 consisting of an amino acid sequence at amino acid positions 51-57 of SEQ ID NO: 16, and a CDR3 consisting of an amino acid sequence at amino acid positions 90-100 of SEQ ID NO: 16;
(c) a heavy chain variable region containing a CDR1 consisting of an amino acid sequence at amino acid positions 31-35 of SEQ ID NO: 18, a CDR2 consisting of an amino acid sequence at amino acid positions 50-65 of SEQ ID NO: 18, and a CDR3 consisting of an amino acid sequence at amino acid positions 98-107 of SEQ ID NO: 18, and a light chain variable region containing a CDR1 consisting of an amino acid sequence at amino acid positions 23-35 of SEQ ID NO: 20, a CDR2 consisting of an amino acid sequence at amino acid positions 51-57 of SEQ ID NO: 20, and a CDR3 consisting of an amino acid sequence at amino acid positions 90-100 of SEQ ID NO: 20; or
(d) a heavy chain variable region containing a CDR1 consisting of an amino acid sequence at amino acid positions 31-35 of SEQ ID NO: 22, a CDR2 consisting of an amino acid sequence at amino acid positions 50-65 of SEQ ID NO: 22, and a CDR3 consisting of an amino acid sequence at amino acid positions 98-110 of SEQ ID NO: 22, and a light chain variable region containing a CDR1 consisting of an amino acid sequence at amino acid positions 23-35 of SEQ ID NO: 24, a CDR2 consisting of an amino acid sequence at amino acid positions 51-57 of SEQ ID NO: 24, and a CDR3 consisting of an amino acid sequence at amino acid positions 90-100 of SEQ ID NO: 24.

In one embodiment, the anti-CLDN4/anti-CD137 bispecific antibody of the present invention contains a heavy chain variable region and a light chain variable region of an anti-CD137 antibody described in any of the following (a) to (i):
(a) a heavy chain variable region consisting of an amino acid sequence at amino acid positions 1-118 of SEQ ID NO: 10 and a light chain variable region consisting of an amino acid sequence at amino acid positions 1-109 of SEQ ID NO: 12;
(b) a heavy chain variable region consisting of an amino acid sequence at amino acid positions 1-118 of SEQ ID NO: 14 and a light chain variable region consisting of an amino acid sequence at amino acid positions 1-111 of SEQ ID NO: 16;
(c) a heavy chain variable region consisting of an amino acid sequence at amino acid positions 1-118 of SEQ ID NO: 18 and a light chain variable region consisting of an amino acid sequence at amino acid positions 1-111 of SEQ ID NO: 20;
(d) a heavy chain variable region consisting of an amino acid sequence at amino acid positions 1-121 of SEQ ID NO: 22 and a light chain variable region consisting of an amino acid sequence at amino acid positions 1-111 of SEQ ID NO: 24;
(e) a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26;
(f) a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28;
(g) a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30;
(h) a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32; or
(i) a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34.

The anti-CLDN4/anti-CD137 bispecific antibody of the present invention may contain, as an anti-CD137 antibody, a full length antibody such as an IgG antibody, or may contain an antigen binding fragment such as a Fab fragment, a Fab′ fragment, a F(ab′)₂ fragment, or a scFv. In one embodiment, the anti-CD137 antibody contained in the anti-CLDN4/anti-CD137 bispecific antibody is an antigen binding fragment of an anti-CD137 antibody. In one embodiment, the antigen binding fragment of the anti-CD137 antibody contained in the anti-CLDN4/anti-CD137 bispecific antibody is an anti-CD137 scFv.

In one embodiment, the anti-CLDN4/anti-CD137 bispecific antibody of the present invention contains an anti-CD137 scFv containing a heavy chain variable region and a light chain variable region of an anti-CD137 antibody described in the following (a) to (m):
(a) a heavy chain variable region containing a CDR1 consisting of an amino acid sequence at amino acid positions 31-35 of SEQ ID NO: 10, a CDR2 consisting of an amino acid sequence at amino acid positions 50-66 of SEQ ID NO: 10, and a CDR3 consisting of an amino acid sequence at amino acid positions 99-107 of SEQ ID NO: 10, and a light chain variable region containing a CDR1 consisting of an amino acid sequence at amino acid positions 24-34 of SEQ ID NO: 12, a CDR2 consisting of an amino acid sequence at amino acid positions 50-56 of SEQ ID NO: 12, and a CDR3 consisting of an amino acid sequence at amino acid positions 89-98 of SEQ ID NO: 12;
(b) a heavy chain variable region containing a CDR1 consisting of an amino acid sequence at amino acid positions 31-35 of SEQ ID NO: 14, a CDR2 consisting of an amino acid sequence at amino acid positions 50-65 of SEQ ID NO: 14, and a CDR3 consisting of an amino acid sequence at amino acid positions 98-107 of SEQ ID NO: 14, and a light chain variable region containing a CDR1 consisting of an amino acid sequence at amino acid positions 23-35 of SEQ ID NO: 16, a CDR2 consisting of an amino acid sequence at amino acid positions 51-57 of SEQ ID NO: 16, and a CDR3 consisting of an amino acid sequence at amino acid positions 90-100 of SEQ ID NO: 16;
(c) a heavy chain variable region containing a CDR1 consisting of an amino acid sequence at amino acid positions 31-35 of SEQ ID NO: 18, a CDR2 consisting of an amino acid sequence at amino acid positions 50-65 of SEQ ID NO: 18, and a CDR3 consisting of an amino acid sequence at amino acid positions 98-107 of SEQ ID NO: 18, and a light chain variable region containing a CDR1 consisting of an amino acid sequence at amino acid positions 23-35 of SEQ ID NO: 20, a CDR2 consisting of an amino acid sequence at amino acid positions 51-57 of SEQ ID NO: 20, and a CDR3 consisting of an amino acid sequence at amino acid positions 90-100 of SEQ ID NO: 20;
(d) a heavy chain variable region containing a CDR1 consisting of an amino acid sequence at amino acid positions 31-35 of SEQ ID NO: 22, a CDR2 consisting of an amino acid sequence at amino acid positions 50-65 of SEQ ID NO: 22, and a CDR3 consisting of an amino acid sequence at amino acid positions 98-110 of SEQ ID NO: 22, and a light chain variable region containing a CDR1 consisting of an amino acid sequence at amino acid positions 23-35 of SEQ ID NO: 24, a CDR2 consisting of an amino acid sequence at amino acid positions 51-57 of SEQ ID NO: 24, and a CDR3 consisting of an amino acid sequence at amino acid positions 90-100 of SEQ ID NO: 24;
(e) a heavy chain variable region consisting of an amino acid sequence at amino acid positions 1-118 of SEQ ID NO: 10 and a light chain variable region consisting of an amino acid sequence at amino acid positions 1-109 of SEQ ID NO: 12;
(f) a heavy chain variable region consisting of an amino acid sequence at amino acid positions 1-118 of SEQ ID NO: 14 and a light chain variable region consisting of an amino acid sequence at amino acid positions 1-111 of SEQ ID NO: 16;
(g) a heavy chain variable region consisting of an amino acid sequence at amino acid positions 1-118 of SEQ ID NO: 18 and a light chain variable region consisting of an amino acid sequence at amino acid positions 1-111 of SEQ ID NO: 20;
(h) a heavy chain variable region consisting of an amino acid sequence at amino acid positions 1-121 of SEQ ID NO: 22 and a light chain variable region consisting of an amino acid sequence at amino acid positions 1-111 of SEQ ID NO: 24;
(i) a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26;
(j) a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28;
(k) a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30;
(1) a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32; or
(m) a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34.

The type and the length of a linker used for linking the heavy chain variable region and the light chain variable region of the anti-CD137 antibody in the anti-CD137 scFv are not especially limited, and can be appropriately selected by those skilled in the art. As the linker, a peptide linker may be used. A preferable length is 5 or more amino acids (with an upper limit being not especially limited, but being usually 30 or less amino acids, and preferably 20 or less amino acids), and is particularly preferably 15 amino acids. As the linker, for example, a glycine-serine linker (GS linker), or a glycine-lysine-proline-glycine-serine linker (GKPGS linker) (SEQ ID NO: 55) can be used. Examples of such linkers include the following:

Ser
Gly-Ser
Gly-Gly-Ser
Ser-Gly-Gly
(SEQ ID No. 46)
Gly-Gly-Gly-Ser
(SEQ ID No. 47)
Ser-Gly-Gly-Gly
(SEQ ID No. 48)
Gly-Gly-Gly-Gly-Ser
(SEQ ID No. 49)
Ser-Gly-Gly-Gly-Gly
(SEQ ID No. 50)
Gly-Gly-Gly-Gly-Gly-Ser
(SEQ ID No. 51)
Ser-Gly-Gly-Gly-Gly-Gly
(SEQ ID No. 52)
Gly-Gly-Gly-Gly-Gly-Gly-Ser
(SEQ ID No. 53)
Ser-Gly-Gly-Gly-Gly-Gly-Gly
(SEQ ID No. 54)
Gly-Gly-Gly-Gly-Ser-Gly-Gly-Gly-Gly-Ser
(SEQ ID NO: 56)
(Gly-Gly-Gly-Gly-Ser)n
(SEQ ID NO: 57)
(Ser-Gly-Gly-Gly-Gly)n
(SEQ ID No. 55)
Gly-Lys-Pro-Gly-Ser
(SEQ ID NO: 58)
(Gly-Lys-Pro-Gly-Ser)n

In the above-described examples, n represents an integer of 1 or more. In one aspect, n is 1 to 10, 2 to 8, or 2 to 6. The length and the sequence of the linker can be appropriately selected depending on purpose by those skilled in the art. In one embodiment, the linker used in the anti-CD137 scFv is a GS linker. In one embodiment, the linker used in the anti-CD137 scFv is a GS linker of (Gly-Gly-Gly-Gly-Ser) n, and n is either 3 (SEQ ID NO: 59) or 4 (SEQ ID NO: 60).

In one embodiment, the anti-CLDN4/anti-CD137 bispecific antibody of the present invention contains an anti-CD137 scFv described in any of the following (a) to (e): (a) an anti-CD137 scFv consisting of an amino acid sequence at amino acid positions 1-242 of SEQ ID NO: 26;
(b) an anti-CD137 scFv consisting of an amino acid sequence at amino acid positions 1-244 of SEQ ID NO: 28;
(c) an anti-CD137 scFv consisting of an amino acid sequence at amino acid positions 1-249 of SEQ ID NO: 30;
(d) an anti-CD137 scFv consisting of an amino acid sequence at amino acid positions 1-244 of SEQ ID NO: 32; or
(e) an anti-CD137 scFv consisting of an amino acid sequence at amino acid positions 1-247 of SEQ ID NO: 34.

In the anti-CLDN4/anti-CD137 bispecific antibody of the present invention, the anti-CLDN4 antibody or its antigen binding fragment may be linked to the anti-CD137 antibody or its antigen binding fragment via a linker. The type and the length of the linker for linking the anti-CLDN4 antibody or its antigen binding fragment to the anti-CD137 antibody or its antigen binding fragment are not especially limited, and can be appropriately selected by those skilled in the art. As the linker, a peptide linker may be used. A preferable length is 5 or more amino acids (with an upper limit being not especially limited, but being usually 30 or less amino acids, and preferably 20 or less amino acids), and is particularly preferably 10 amino acids. As the peptide linker, for example, a glycine-serine linker (GS linker), or a glycine-lysine-proline-glycine-serine linker (GKPGS linker) (SEQ ID NO: 55) can be used. Examples of such linkers include the following:

Ser
Gly-Ser
Gly-Gly-Ser
Ser-Gly-Gly
(SEQ ID No. 46)
Gly-Gly-Gly-Ser
(SEQ ID No. 47)
Ser-Gly-Gly-Gly
(SEQ ID No. 48)
Gly-Gly-Gly-Gly-Ser
(SEQ ID No. 49)
Ser-Gly-Gly-Gly-Gly
(SEQ ID No. 50)
Gly-Gly-Gly-Gly-Gly-Ser
(SEQ ID No. 51)
Ser-Gly-Gly-Gly-Gly-Gly
(SEQ ID No. 52)
Gly-Gly-Gly-Gly-Gly-Gly-Ser
(SEQ ID No. 53)
Ser-Gly-Gly-Gly-Gly-Gly-Gly
(SEQ ID No. 54)
Gly-Gly-Gly-Gly-Ser-Gly-Gly-Gly-Gly-Ser
(SEQ ID NO: 56)
(Gly-Gly-Gly-Gly-Ser)n
(SEQ ID NO: 57)
(Ser-Gly-Gly-Gly-Gly)n
(SEQ ID No. 55)
Gly-Lys-Pro-Gly-Ser
(SEQ ID NO: 58)
(Gly-Lys-Pro-Gly-Ser)n

In the above-described examples, n represents an integer of 1 or more. In one aspect, n is 1 to 10, 2 to 8, or 2 to 6. The length and the sequence of the peptide linker can be appropriately selected depending on purpose by those skilled in the art. In one embodiment, a linker used as the linker for linking the anti-CLDN4 antibody or its antigen binding fragment to the anti-CD137 antibody or its antigen binding fragment is a GS linker or a GKPGS linker (SEQ ID NO: 55), and in another embodiment, is a linker having an amino acid sequence of SEQ ID NO: 54.

In one embodiment, the anti-CLDN4/anti-CD137 bispecific antibody of the present invention contains an anti-CLDN4 IgG antibody and an anti-CD137 scFv, and has a structure in which the anti-CLDN4 IgG antibody is linked to the anti-CD137 scFv via a linker. The linkage site between the anti-CLDN4 IgG antibody and the anti-CD137 scFv is not especially limited, and for example, the anti-CLDN4/anti-CD137 bispecific antibody of the present invention can take various structures, such as the heavy chain amino terminal of the anti-CLDN4 IgG antibody linked to the carboxy terminal of the anti-CD137 scFv, the light chain amino terminal of the anti-CLDN4 IgG antibody linked to the carboxy terminal of the anti-CD137 scFv, the amino terminal of the anti-CD137 scFv linked to the heavy chain carboxy terminal of the anti-CLDN4 IgG antibody, and the amino terminal of the anti-CD137 scFv linked to the light chain carboxy terminal of the anti-CLDN4 IgG antibody. In one embodiment, the anti-CLDN4/anti-CD137 bispecific antibody of the present invention comprises the amino terminal of the anti-CD137 scFv linked to the heavy chain carboxy terminal of the anti-CLDN4 IgG antibody, or the amino terminal of the anti-CD137 scFv linked to the light chain carboxy terminal of the anti-CLDN4 IgG antibody. In one embodiment, the anti-CLDN4/anti-CD137 bispecific antibody of the present invention comprises the amino terminal of the anti-CD137 scFv linked to the heavy chain carboxy terminal of the anti-CLDN4 IgG antibody.

In one embodiment, the anti-CLDN4/anti-CD137 bispecific antibody of the present invention is an anti-CLDN4/anti-CD137 bispecific antibody containing an anti-CLDN4 IgG antibody and an anti-CD137 scFv, in which the amino terminal of the anti-CD137 scFv is linked to a heavy chain or light chain carboxy terminal of the anti-CLDN4 IgG antibody via a linker, and a heavy chain variable region and a light chain variable region of the anti-CD137 scFv are described in any of the following (a) to (m):
(a) a heavy chain variable region containing a CDR1 consisting of an amino acid sequence at amino acid positions 31-35 of SEQ ID NO: 10, a CDR2 consisting of an amino acid sequence at amino acid positions 50-66 of SEQ ID NO: 10, and a CDR3 consisting of an amino acid sequence at amino acid positions 99-107 of SEQ ID NO: 10, and a light chain variable region containing a CDR1 consisting of an amino acid sequence at amino acid positions 24-34 of SEQ ID NO: 12, a CDR2 consisting of an amino acid sequence at amino acid positions 50-56 of SEQ ID NO: 12, and a CDR3 consisting of an amino acid sequence at amino acid positions 89-98 of SEQ ID NO: 12;
(b) a heavy chain variable region containing a CDR1 consisting of an amino acid sequence at amino acid positions 31-35 of SEQ ID NO: 14, a CDR2 consisting of an amino acid sequence at amino acid positions 50-65 of SEQ ID NO: 14, and a CDR3 consisting of an amino acid sequence at amino acid positions 98-107 of SEQ ID NO: 14, and a light chain variable region containing a CDR1 consisting of an amino acid sequence at amino acid positions 23-35 of SEQ ID NO: 16, a CDR2 consisting of an amino acid sequence at amino acid positions 51-57 of SEQ ID NO: 16, and a CDR3 consisting of an amino acid sequence at amino acid positions 90-100 of SEQ ID NO: 16;
(c) a heavy chain variable region containing a CDR1 consisting of an amino acid sequence at amino acid positions 31-35 of SEQ ID NO: 18, a CDR2 consisting of an amino acid sequence at amino acid positions 50-65 of SEQ ID NO: 18, and a CDR3 consisting of an amino acid sequence at amino acid positions 98-107 of SEQ ID NO: 18, and a light chain variable region containing a CDR1 consisting of an amino acid sequence at amino acid positions 23-35 of SEQ ID NO: 20, a CDR2 consisting of an amino acid sequence at amino acid positions 51-57 of SEQ ID NO: 20, and a CDR3 consisting of an amino acid sequence at amino acid positions 90-100 of SEQ ID NO: 20;
(d) a heavy chain variable region containing a CDR1 consisting of an amino acid sequence at amino acid positions 31-35 of SEQ ID NO: 22, a CDR2 consisting of an amino acid sequence at amino acid positions 50-65 of SEQ ID NO: 22, and a CDR3 consisting of an amino acid sequence at amino acid positions 98-110 of SEQ ID NO: 22, and a light chain variable region containing a CDR1 consisting of an amino acid sequence at amino acid positions 23-35 of SEQ ID NO: 24, a CDR2 consisting of an amino acid sequence at amino acid positions 51-57 of SEQ ID NO: 24, and a CDR3 consisting of an amino acid sequence at amino acid positions 90-100 of SEQ ID NO: 24;
(e) a heavy chain variable region consisting of an amino acid sequence at amino acid positions 1-118 of SEQ ID NO: 10 and a light chain variable region consisting of an amino acid sequence at amino acid positions 1-109 of SEQ ID NO: 12;
(f) a heavy chain variable region consisting of an amino acid sequence at amino acid positions 1-118 of SEQ ID NO: 14 and a light chain variable region consisting of an amino acid sequence at amino acid positions 1-111 of SEQ ID NO: 16;
(g) a heavy chain variable region consisting of an amino acid sequence at amino acid positions 1-118 of SEQ ID NO: 18 and a light chain variable region consisting of an amino acid sequence at amino acid positions 1-111 of SEQ ID NO: 20;
(h) a heavy chain variable region consisting of an amino acid sequence at amino acid positions 1-121 of SEQ ID NO: 22 and a light chain variable region consisting of an amino acid sequence at amino acid positions 1-111 of SEQ ID NO: 24;
(i) a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26;
(j) a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28;
(k) a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30;
(1) a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32; or
(m) a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34.

In one embodiment, the anti-CLDN4/anti-CD137 bispecific antibody of the present invention is an anti-CLDN4/anti-CD137 bispecific antibody containing an anti-CLDN4 IgG antibody and an anti-CD137 scFv, in which the amino terminal of the anti-CD137 scFv is linked to a heavy chain or light chain carboxy terminal of the anti-CLDN4 IgG antibody via a linker, and the anti-CD137 scFv is described in any of the following (a) to (e):
(a) an anti-CD137 scFv consisting of an amino acid sequence at amino acid positions 1-242 of SEQ ID NO: 26;
(b) an anti-CD137 scFv consisting of an amino acid sequence at amino acid positions 1-244 of SEQ ID NO: 28;
(c) an anti-CD137 scFv consisting of an amino acid sequence at amino acid positions 1-249 of SEQ ID NO: 30;
(d) an anti-CD137 scFv consisting of an amino acid sequence at amino acid positions 1-244 of SEQ ID NO: 32; or
(e) an anti-CD137 scFv consisting of an amino acid sequence at amino acid positions 1-247 of SEQ ID NO: 34.

In one embodiment, the anti-CLDN4/anti-CD137 bispecific antibody of the present invention is an anti-CLDN4/anti-CD137 scFv bispecific antibody described in any of the following (a) to (h):
(a) a bispecific antibody containing: a heavy chain of an anti-CLDN4 antibody containing a heavy chain variable region containing a CDR1 consisting of an amino acid sequence at amino acid positions 31-35 of SEQ ID NO: 2, a CDR2 consisting of an amino acid sequence at amino acid positions 50-66 of SEQ ID NO: 2, and a CDR3 consisting of an amino acid sequence at amino acid positions 99-114 of SEQ ID NO: 2, and a light chain of an anti-CLDN4 antibody containing a light chain variable region containing a
   CDR1 consisting of an amino acid sequence at amino acid positions 24-35 of SEQ ID NO: 4, a CDR2 consisting of an amino acid sequence at amino acid positions 51-57 of SEQ ID NO: 4, and a CDR3 consisting of an amino acid sequence at amino acid positions 90-98 of SEQ ID NO: 4; and an anti-CD137 scFv containing a heavy chain variable region containing a CDR1 consisting of an amino acid sequence at amino acid positions 31-35 of SEQ ID NO: 10, a CDR2 consisting of an amino acid sequence at amino acid positions 50-66 of SEQ ID NO: 10, and a CDR3 consisting of an amino acid sequence at amino acid positions 99-107 of SEQ ID NO: 10, and a light chain variable region containing a CDR1 consisting of an amino acid sequence at amino acid positions 24-34 of SEQ ID NO: 12, a CDR2 consisting of an amino acid sequence at amino acid positions 50-56 of SEQ ID NO: 12, and a
   CDR3 consisting of an amino acid sequence at amino acid positions 89-98 of SEQ ID NO: 12, in which the amino terminal of the anti-CD137 scFv is linked to the heavy chain carboxy terminal of the anti-CLDN4 antibody via a linker;

(b) a bispecific antibody containing: a heavy chain of an anti-CLDN4 antibody containing a heavy chain variable region containing a CDR1 consisting of an amino acid sequence at amino acid positions 31-35 of SEQ ID NO: 2, a CDR2 consisting of an amino acid sequence at amino acid positions 50-66 of SEQ ID NO: 2, and a CDR3 consisting of an amino acid sequence at amino acid positions 99-114 of SEQ ID NO: 2, and a light chain of an anti-CLDN4 antibody containing a light chain variable region containing a

CDR1 consisting of an amino acid sequence at amino acid positions 24-35 of SEQ ID NO: 4, a CDR2 consisting of an amino acid sequence at amino acid positions 51-57 of SEQ ID NO: 4, and a CDR3 consisting of an amino acid sequence at amino acid positions 90-98 of SEQ ID NO: 4; and an anti-CD137 scFv containing a heavy chain variable region containing a CDR1 consisting of an amino acid sequence at amino acid positions 31-35 of SEQ ID NO: 14, a CDR2 consisting of an amino acid sequence at amino acid positions 50-65 of SEQ ID NO: 14, and a CDR3 consisting of an amino acid sequence at amino acid positions 98-107 of SEQ ID NO: 14, and a light chain variable region containing a CDR1 consisting of an amino acid sequence at amino acid positions 23-35 of SEQ ID NO: 16, a CDR2 consisting of an amino acid sequence at amino acid positions 51-57 of SEQ ID NO: 16, and a
CDR3 consisting of an amino acid sequence at amino acid positions 90-100 of SEQ ID NO: 16, in which the amino terminal of the anti-CD137 scFv is linked to the heavy chain carboxy terminal of the anti-CLDN4 antibody via a linker;
(c) a bispecific antibody containing: a heavy chain of an anti-CLDN4 antibody containing a heavy chain variable region containing a CDR1 consisting of an amino acid sequence at amino acid positions 31-35 of SEQ ID NO: 2, a CDR2 consisting of an amino acid sequence at amino acid positions 50-66 of SEQ ID NO: 2, and a CDR3 consisting of an amino acid sequence at amino acid positions 99-114 of SEQ ID NO: 2, and a light chain of an anti-CLDN4 antibody containing a light chain variable region containing a
   CDR1 consisting of an amino acid sequence at amino acid positions 24-35 of SEQ ID NO: 4, a CDR2 consisting of an amino acid sequence at amino acid positions 51-57 of SEQ ID NO: 4, and a CDR3 consisting of an amino acid sequence at amino acid positions 90-98 of SEQ ID NO: 4; and an anti-CD137 scFv containing a heavy chain variable region containing a CDR1 consisting of an amino acid sequence at amino acid positions 31-35 of SEQ ID NO: 18, a CDR2 consisting of an amino acid sequence at amino acid positions 50-65 of SEQ ID NO: 18, and a CDR3 consisting of an amino acid sequence at amino acid positions 98-107 of SEQ ID NO: 18, and a light chain variable region containing a CDR1 consisting of an amino acid sequence at amino acid positions 23-35 of SEQ ID NO: 20, a CDR2 consisting of an amino acid sequence at amino acid positions 51-57 of SEQ ID NO: 20, and a
   CDR3 consisting of an amino acid sequence at amino acid positions 90-100 of SEQ ID NO: 20, in which the amino terminal of the anti-CD137 scFv is linked to the heavy chain carboxy terminal of the anti-CLDN4 antibody via a linker;

(d) a bispecific antibody containing: a heavy chain of an anti-CLDN4 antibody containing a heavy chain variable region containing a CDR1 consisting of an amino acid sequence at amino acid positions 31-35 of SEQ ID NO: 2, a CDR2 consisting of an amino acid sequence at amino acid positions 50-66 of SEQ ID NO: 2, and a CDR3 consisting of an amino acid sequence at amino acid positions 99-114 of SEQ ID NO: 2, and a light chain of an anti-CLDN4 antibody containing a light chain variable region containing a

CDR1 consisting of an amino acid sequence at amino acid positions 24-35 of SEQ ID NO: 4, a CDR2 consisting of an amino acid sequence at amino acid positions 51-57 of SEQ ID NO: 4, and a CDR3 consisting of an amino acid sequence at amino acid positions 90-98 of SEQ ID NO: 4; and an anti-CD137 scFv containing a heavy chain variable region containing a CDR1 consisting of an amino acid sequence at amino acid positions 31-35 of SEQ ID NO: 22, a CDR2 consisting of an amino acid sequence at amino acid positions 50-65 of SEQ ID NO: 22, and a CDR3 consisting of an amino acid sequence at amino acid positions 98-110 of SEQ ID NO: 22, and a light chain variable region containing a CDR1 consisting of an amino acid sequence at amino acid positions 23-35 of SEQ ID NO: 24, a CDR2 consisting of an amino acid sequence at amino acid positions 51-57 of SEQ ID NO: 24, and a
CDR3 consisting of an amino acid sequence at amino acid positions 90-100 of SEQ ID NO: 24, in which the amino terminal of the anti-CD137 scFv is linked to the heavy chain carboxy terminal of the anti-CLDN4 antibody via a linker;

(e) a bispecific antibody containing: a heavy chain of an anti-CLDN4 antibody containing a heavy chain variable region containing a CDR1 consisting of an amino acid sequence at amino acid positions 31-35 of SEQ ID NO: 6, a CDR2 consisting of an amino acid sequence at amino acid positions 50-66 of SEQ ID NO: 6, and a CDR3 consisting of an amino acid sequence at amino acid positions 99-112 of SEQ ID NO: 6, and a light chain of an anti-CLDN4 antibody containing a light chain variable region containing a

CDR1 consisting of an amino acid sequence at amino acid positions 24-35 of SEQ ID NO: 8, a CDR2 consisting of an amino acid sequence at amino acid positions 51-57 of SEQ ID NO: 8, and a CDR3 consisting of an amino acid sequence at amino acid positions 90-98 of SEQ ID NO: 8; and an anti-CD137 scFv containing a heavy chain variable region containing a CDR1 consisting of an amino acid sequence at amino acid positions 31-35 of SEQ ID NO: 10, a CDR2 consisting of an amino acid sequence at amino acid positions 50-66 of SEQ ID NO: 10, and a CDR3 consisting of an amino acid sequence at amino acid positions 99-107 of SEQ ID NO: 10, and a light chain variable region containing a CDR1 consisting of an amino acid sequence at amino acid positions 24-34 of SEQ ID NO: 12, a CDR2 consisting of an amino acid sequence at amino acid positions 50-56 of SEQ ID NO: 12, and a
CDR3 consisting of an amino acid sequence at amino acid positions 89-98 of SEQ ID NO: 12, in which the amino terminal of the anti-CD137 scFv is linked to the heavy chain carboxy terminal of the anti-CLDN4 antibody via a linker;

(f) a bispecific antibody containing: a heavy chain of an anti-CLDN4 antibody containing a heavy chain variable region containing a CDR1 consisting of an amino acid sequence at amino acid positions 31-35 of SEQ ID NO: 6, a CDR2 consisting of an amino acid sequence at amino acid positions 50-66 of SEQ ID NO: 6, and a CDR3 consisting of an amino acid sequence at amino acid positions 99-112 of SEQ ID NO: 6, and a light chain of an anti-CLDN4 antibody containing a light chain variable region containing a

CDR1 consisting of an amino acid sequence at amino acid positions 24-35 of SEQ ID NO: 8, a CDR2 consisting of an amino acid sequence at amino acid positions 51-57 of SEQ ID NO: 8, and a CDR3 consisting of an amino acid sequence at amino acid positions 90-98 of SEQ ID NO: 8; and an anti-CD137 scFv containing a heavy chain variable region containing a CDR1 consisting of an amino acid sequence at amino acid positions 31-35 of SEQ ID NO: 14, a CDR2 consisting of an amino acid sequence at amino acid positions 50-65 of SEQ ID NO: 14, and a CDR3 consisting of an amino acid sequence at amino acid positions 98-107 of SEQ ID NO: 14, and a light chain variable region containing a CDR1 consisting of an amino acid sequence at amino acid positions 23-35 of SEQ ID NO: 16, a CDR2 consisting of an amino acid sequence at amino acid positions 51-57 of SEQ ID NO: 16, and a
CDR3 consisting of an amino acid sequence at amino acid positions 90-100 of SEQ ID NO: 16, in which the amino terminal of the anti-CD137 scFv is linked to the heavy chain carboxy terminal of the anti-CLDN4 antibody via a linker;

(g) a bispecific antibody containing: a heavy chain of an anti-CLDN4 antibody containing a heavy chain variable region containing a CDR1 consisting of an amino acid sequence at amino acid positions 31-35 of SEQ ID NO: 6, a CDR2 consisting of an amino acid sequence at amino acid positions 50-66 of SEQ ID NO: 6, and a CDR3 consisting of an amino acid sequence at amino acid positions 99-112 of SEQ ID NO: 6, and a light chain of an anti-CLDN4 antibody containing a light chain variable region containing a

CDR1 consisting of an amino acid sequence at amino acid positions 24-35 of SEQ ID NO: 8, a CDR2 consisting of an amino acid sequence at amino acid positions 51-57 of SEQ ID NO: 8, and a CDR3 consisting of an amino acid sequence at amino acid positions 90-98 of SEQ ID NO: 8; and an anti-CD137 scFv containing a heavy chain variable region containing a CDR1 consisting of an amino acid sequence at amino acid positions 31-35 of SEQ ID NO: 18, a CDR2 consisting of an amino acid sequence at amino acid positions 50-65 of SEQ ID NO: 18, and a CDR3 consisting of an amino acid sequence at amino acid positions 98-107 of SEQ ID NO: 18, and a light chain variable region containing a CDR1 consisting of an amino acid sequence at amino acid positions 23-35 of SEQ ID NO: 20, a CDR2 consisting of an amino acid sequence at amino acid positions 51-57 of SEQ ID NO: 20, and a
CDR3 consisting of an amino acid sequence at amino acid positions 90-100 of SEQ ID NO: 20, in which the amino terminal of the anti-CD137 scFv is linked to the heavy chain carboxy terminal of the anti-CLDN4 antibody via a linker; or

(h) a bispecific antibody containing: a heavy chain of an anti-CLDN4 antibody containing a heavy chain variable region containing a CDR1 consisting of an amino acid sequence at amino acid positions 31-35 of SEQ ID NO: 6, a CDR2 consisting of an amino acid sequence at amino acid positions 50-66 of SEQ ID NO: 6, and a CDR3 consisting of an amino acid sequence at amino acid positions 99-112 of SEQ ID NO: 6, and a light chain of an anti-CLDN4 antibody containing a light chain variable region containing a

CDR1 consisting of an amino acid sequence at amino acid positions 24-35 of SEQ ID NO: 8, a CDR2 consisting of an amino acid sequence at amino acid positions 51-57 of SEQ ID NO: 8, and a CDR3 consisting of an amino acid sequence at amino acid positions 90-98 of SEQ ID NO: 8; and an anti-CD137 scFv containing a heavy chain variable region containing a CDR1 consisting of an amino acid sequence at amino acid positions 31-35 of SEQ ID NO: 22, a CDR2 consisting of an amino acid sequence at amino acid positions 50-65 of SEQ ID NO: 22, and a CDR3 consisting of an amino acid sequence at amino acid positions 98-110 of SEQ ID NO: 22, and a light chain variable region containing a CDR1 consisting of an amino acid sequence at amino acid positions 23-35 of SEQ ID NO: 24, a CDR2 consisting of an amino acid sequence at amino acid positions 51-57 of SEQ ID NO: 24, and a
CDR3 consisting of an amino acid sequence at amino acid positions 90-100 of SEQ ID NO: 24, in which the amino terminal of the anti-CD137 scFv is linked to the heavy chain carboxy terminal of the anti-CLDN4 antibody via a linker.

In one embodiment, the anti-CLDN4/anti-CD137 bispecific antibody of the present invention is an anti-CLDN4/anti-CD137 scFv bispecific antibody described in any of the following (a) to (j):
(a) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26, wherein an amino terminal of the anti-CD137 scFv is linked to a heavy chain carboxy terminal of the anti-CLDN4 antibody via a linker;
(b) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28, wherein an amino terminal of the anti-CD137 scFv is linked to a heavy chain carboxy terminal of the anti-CLDN4 antibody via a linker;
(c) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30, wherein an amino terminal of the anti-CD137 scFv is linked to a heavy chain carboxy terminal of the anti-CLDN4 antibody via a linker;
(d) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32, wherein an amino terminal of the anti-CD137 scFv is linked to a heavy chain carboxy terminal of the anti-CLDN4 antibody via a linker;
(e) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34, wherein an amino terminal of the anti-CD137 scFv is linked to a heavy chain carboxy terminal of the anti-CLDN4 antibody via a linker;
(f) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26, wherein an amino terminal of the anti-CD137 scFv is linked to a heavy chain carboxy terminal of the anti-CLDN4 antibody via a linker;
(g) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28, wherein an amino terminal of the anti-CD137 scFv is linked to a heavy chain carboxy terminal of the anti-CLDN4 antibody via a linker;
(h) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30, wherein an amino terminal of the anti-CD137 scFv is linked to a heavy chain carboxy terminal of the anti-CLDN4 antibody via a linker;
(i) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32, wherein an amino terminal of the anti-CD137 scFv is linked to a heavy chain carboxy terminal of the anti-CLDN4 antibody via a linker;
(j) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34, wherein an amino terminal of the anti-CD137 scFv is linked to a heavy chain carboxy terminal of the anti-CLDN4 antibody via a linker;
(k) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26, wherein an amino terminal of the anti-CD137 scFv is linked to a light chain carboxy terminal of the anti-CLDN4 antibody via a linker;
(l) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28, wherein an amino terminal of the anti-CD137 scFv is linked to a light chain carboxy terminal of the anti-CLDN4 antibody via a linker;
(m) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30, wherein an amino terminal of the anti-CD137 scFv is linked to a light chain carboxy terminal of the anti-CLDN4 antibody via a linker;
(n) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32, wherein an amino terminal of the anti-CD137 scFv is linked to a light chain carboxy terminal of the anti-CLDN4 antibody via a linker;
(o) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34, wherein an amino terminal of the anti-CD137 scFv is linked to a light chain carboxy terminal of the anti-CLDN4 antibody via a linker;
(p) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26, wherein an amino terminal of the anti-CD137 scFv is linked to a light chain carboxy terminal of the anti-CLDN4 antibody via a linker;
(q) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28, wherein an amino terminal of the anti-CD137 scFv is linked to a light chain carboxy terminal of the anti-CLDN4 antibody via a linker;
(r) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30, wherein an amino terminal of the anti-CD137 scFv is linked to a light chain carboxy terminal of the anti-CLDN4 antibody via a linker;
(s) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32, wherein an amino terminal of the anti-CD137 scFv is linked to a light chain carboxy terminal of the anti-CLDN4 antibody via a linker; or
(t) a bispecific antibody containing a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34, in which the amino terminal of the anti-CD137 scFv is linked to the light chain carboxy terminal of the anti-CLDN4 antibody via a linker.

In one embodiment, the anti-CLDN4/anti-CD137 bispecific antibody of the present invention is an anti-CLDN4/anti-CD137 scFv bispecific antibody described in the following (a) or (b):
(a) a bispecific antibody containing a polypeptide consisting of an amino acid sequence at amino acid positions 1-705 of SEQ ID NO: 36 containing a heavy chain of an anti-CLDN4 antibody and an anti-CD137 scFv, and a polypeptide containing a light chain of an anti-CLDN4 antibody consisting of an amino acid sequence at amino acid positions 1-215 of SEQ ID NO: 40; or
(b) a bispecific antibody containing a polypeptide consisting of an amino acid sequence at amino acid positions 1-712 of SEQ ID NO: 38 containing a heavy chain of an anti-CLDN4 antibody and an anti-CD137 scFv, and a light chain of an anti-CLDN4 antibody consisting of an amino acid sequence at amino acid positions 1-215 of SEQ ID NO: 40.

Herein, the term “post-translational modification” refers to that an antibody expressed in a cell is modified after translation. Examples of the post-translational modification include modification such as pyroglutamylation, glycosylation, oxidation, deamidation or glycation of glutamine or glutamic acid at the heavy chain N-terminal, and lysine deletion by cutting lysine at the heavy chain C-terminal with carboxypeptidase. It is known that such post-translational modification is caused in various antibodies (J. Pharm. Sci., 2008, Vol. 97, p. 2426-2447).

In one embodiment, the anti-CLDN4/anti-CD137 bispecific antibody of the present invention may be post-translationally modified. In one embodiment, the post-translational modification is pyroglutamylation at the N-terminal of the heavy chain variable region and/or lysine deletion at the heavy chain C-terminal. It is known in this field that the post-translational modification by pyroglutamylation at the N-terminal or lysine deletion at the C-terminal does not affect the activity of the antibody (Analytical Biochemistry, 2006, Vol. 348, p. 24-39).

The anti-CLDN4/anti-CD137 bispecific antibody of the present invention binds to human CLDN4 and human CD137. It can be checked by employing a known binding activity measurement method whether or not the antibody binds to human CLDN4 and human CD137. Examples of a method for measuring binding activity include a method such as ELISA and flow cytometry. ELISA or flow cytometry can be carried out by a method usually employed by those skilled in the art, and a method described in, for example, Example 3 or 5 can be employed.

The anti-CLDN4/anti-CD137 bispecific antibody of the present invention can be easily produced by those skilled in the art with human CLDN4 and human CD137 used as antigens by employing an antibody production technique known in this field, or can be easily produced by those skilled in the art based on sequence information and the like of the heavy chain variable regions and the light chain variable regions of the anti-CLDN4 antibody and the anti-CD137 antibody disclosed herein by employing a method known in this field.

The anti-CLDN4/anti-CD137 bispecific antibody of the present invention can be produced in accordance with, for example, a method described in <Method for Producing Bispecific Antibody of Invention> described below, although not especially limited thereto.

### <Polynucleotide of Bispecific Antibody of Invention>

The present invention also provides a polynucleotide usable in production of the anti-CLDN4/anti-CD137 bispecific antibody of the present invention (also referred to as the “polynucleotide of the bispecific antibody of the present invention”).

In one embodiment, the polynucleotide of the bispecific antibody of the present invention is a polynucleotide having a nucleotide sequence encoding a heavy chain variable region or light chain variable region of an anti-CLDN4 antibody, which is selected from the group consisting of the following (a) to (d):
(a) a polynucleotide having a nucleotide sequence encoding a heavy chain variable region of an anti-CLDN4 antibody consisting of an amino acid sequence at amino acid positions 1-125 of SEQ ID NO: 2;
(b) a polynucleotide having a nucleotide sequence encoding a light chain variable region of an anti-CLDN4 antibody consisting of an amino acid sequence at amino acid positions 1-109 of SEQ ID NO: 4;
(c) a polynucleotide having a nucleotide sequence encoding a heavy chain variable region of an anti-CLDN4 antibody consisting of an amino acid sequence at amino acid positions 1-123 of SEQ ID NO: 6; or
(d) a polynucleotide having a nucleotide sequence encoding a light chain variable region of an anti-CLDN4 antibody consisting of an amino acid sequence at amino acid positions 1-109 of SEQ ID NO: 8.

In one embodiment, the polynucleotide of the bispecific antibody of the present invention is a polynucleotide having a nucleotide sequence encoding a heavy chain variable region and a light chain variable region of an anti-CLDN4 antibody, which is selected from the following (a) or (b):
(a) a polynucleotide having a nucleotide sequence encoding a heavy chain variable region of an anti-CLDN4 antibody consisting of an amino acid sequence at amino acid positions 1-125 of SEQ ID NO: 2, and a polynucleotide having a nucleotide sequence encoding a light chain variable region of an anti-CLDN4 antibody consisting of an amino acid sequence at amino acid positions 1-109 of SEQ ID NO: 4; or
(b) a polynucleotide having a nucleotide sequence encoding a heavy chain variable region of an anti-CLDN4 antibody consisting of an amino acid sequence at amino acid positions 1-123 of SEQ ID NO: 6, and a polynucleotide having a nucleotide sequence encoding a light chain variable region of an anti-CLDN4 antibody consisting of an amino acid sequence at amino acid positions 1-109 of SEQ ID NO: 8.

In one embodiment, the polynucleotide of the bispecific antibody of the present invention is a polynucleotide having a nucleotide sequence encoding a heavy chain variable region or light chain variable region of an anti-CD137 antibody, which is selected from the group consisting of the following (a) to (r):
(a) a polynucleotide having a nucleotide sequence encoding a heavy chain variable region of an anti-CD137 antibody consisting of an amino acid sequence at amino acid positions 1-118 of SEQ ID NO: 10;
(b) a polynucleotide having a nucleotide sequence encoding a light chain variable region of an anti-CD137 antibody consisting of an amino acid sequence at amino acid positions 1-109 of SEQ ID NO: 12;
(c) a polynucleotide having a nucleotide sequence encoding a heavy chain variable region of an anti-CD137 antibody consisting of an amino acid sequence at amino acid positions 1-118 of SEQ ID NO: 14;
(d) a polynucleotide having a nucleotide sequence encoding a light chain variable region of an anti-CD137 antibody consisting of an amino acid sequence at amino acid positions 1-111 of SEQ ID NO: 16;
(e) a polynucleotide having a nucleotide sequence encoding a heavy chain variable region of an anti-CD137 antibody consisting of an amino acid sequence at amino acid positions 1-118 of SEQ ID NO: 18;
(f) a polynucleotide having a nucleotide sequence encoding a light chain variable region of an anti-CD137 antibody consisting of an amino acid sequence at amino acid positions 1-111 of SEQ ID NO: 20;
(g) a polynucleotide having a nucleotide sequence encoding a heavy chain variable region of an anti-CD137 antibody consisting of an amino acid sequence at amino acid positions 1-121 of SEQ ID NO: 22;
(h) a polynucleotide having a nucleotide sequence encoding a light chain variable region of an anti-CD137 antibody consisting of an amino acid sequence at amino acid positions 1-111 of SEQ ID NO: 24;
(i) a polynucleotide having a nucleotide sequence encoding a heavy chain variable region of an anti-CD137 antibody consisting of an amino acid sequence at amino acid positions 1-118 of SEQ ID NO: 26;
(j) a polynucleotide having a nucleotide sequence encoding a light chain variable region of an anti-CD137 antibody consisting of an amino acid sequence at amino acid positions 134-242 of SEQ ID NO: 26;
(k) a polynucleotide having a nucleotide sequence encoding a heavy chain variable region of an anti-CD137 antibody consisting of an amino acid sequence at amino acid positions 1-118 of SEQ ID NO: 28;
(l) a polynucleotide having a nucleotide sequence encoding a light chain variable region of an anti-CD137 antibody consisting of an amino acid sequence at amino acid positions 134-244 of SEQ ID NO: 28;
(m) a polynucleotide having a nucleotide sequence encoding a light chain variable region of an anti-CD137 antibody consisting of an amino acid sequence at amino acid positions 1-111 of SEQ ID NO: 30;
(n) a polynucleotide having a nucleotide sequence encoding a heavy chain variable region of an anti-CD137 antibody consisting of an amino acid sequence at amino acid positions 132-249 of SEQ ID NO: 30;
(o) a polynucleotide having a nucleotide sequence encoding a heavy chain variable region of an anti-CD137 antibody consisting of an amino acid sequence at amino acid positions 1-118 of SEQ ID NO: 32;
(p) a polynucleotide having a nucleotide sequence encoding a light chain variable region of an anti-CD137 antibody consisting of an amino acid sequence at amino acid positions 134-244 of SEQ ID NO: 32;
(q) a polynucleotide having a nucleotide sequence encoding a heavy chain variable region of an anti-CD137 antibody consisting of an amino acid sequence at amino acid positions 1-121 of SEQ ID NO: 34; or
(r) a polynucleotide having a nucleotide sequence encoding a light chain variable region of an anti-CD137 antibody consisting of an amino acid sequence at amino acid positions 137-247 of SEQ ID NO: 34.

In one embodiment, the polynucleotide of the bispecific antibody of the present invention is a polynucleotide having a nucleotide sequence encoding a heavy chain variable region and a light chain variable region of an anti-CD137 antibody, which is selected from the group consisting of the following (a) to (i):
(a) a polynucleotide having a nucleotide sequence encoding a heavy chain variable region of an anti-CD137 antibody consisting of an amino acid sequence at amino acid positions 1-118 of SEQ ID NO: 10, and a polynucleotide having a nucleotide sequence encoding a light chain variable region of an anti-CD137 antibody consisting of an amino acid sequence at amino acid positions 1-109 of SEQ ID NO: 12;
(b) a polynucleotide having a nucleotide sequence encoding a heavy chain variable region of an anti-CD137 antibody consisting of an amino acid sequence at amino acid positions 1-118 of SEQ ID NO: 14, and a polynucleotide having a nucleotide sequence encoding a light chain variable region of an anti-CD137 antibody consisting of an amino acid sequence at amino acid positions 1-111 of SEQ ID NO: 16;
(c) a polynucleotide having a nucleotide sequence encoding a heavy chain variable region of an anti-CD137 antibody consisting of an amino acid sequence at amino acid positions 1-118 of SEQ ID NO: 18, and a polynucleotide having a nucleotide sequence encoding a light chain variable region of an anti-CD137 antibody consisting of an amino acid sequence at amino acid positions 1-111 of SEQ ID NO: 20;
(d) a polynucleotide having a nucleotide sequence encoding a heavy chain variable region of an anti-CD137 antibody consisting of an amino acid sequence at amino acid positions 1-121 of SEQ ID NO: 22, and a polynucleotide having a nucleotide sequence encoding a light chain variable region of an anti-CD137 antibody consisting of an amino acid sequence at amino acid positions 1-111 of SEQ ID NO: 24;
(e) a polynucleotide having a nucleotide sequence encoding a heavy chain variable region of an anti-CD137 antibody consisting of an amino acid sequence at amino acid positions 1-118 of SEQ ID NO: 26, and a polynucleotide having a nucleotide sequence encoding a light chain variable region of an anti-CD137 antibody consisting of an amino acid sequence at amino acid positions 134-242 of SEQ ID NO: 26;
(f) a polynucleotide having a nucleotide sequence encoding a heavy chain variable region of an anti-CD137 antibody consisting of an amino acid sequence at amino acid positions 1-118 of SEQ ID NO: 28, and a polynucleotide having a nucleotide sequence encoding a light chain variable region of an anti-CD137 antibody consisting of an amino acid sequence at amino acid positions 134-244 of SEQ ID NO: 28;
(g) a polynucleotide having a nucleotide sequence encoding a light chain variable region of an anti-CD137 antibody consisting of an amino acid sequence at amino acid positions 1-111 of SEQ ID NO: 30, and a polynucleotide having a nucleotide sequence encoding a heavy chain variable region of an anti-CD137 antibody consisting of an amino acid sequence at amino acid positions 132-249 of SEQ ID NO: 30;
(h) a polynucleotide having a nucleotide sequence encoding a heavy chain variable region of an anti-CD137 antibody consisting of an amino acid sequence at amino acid positions 1-118 of SEQ ID NO: 32, and a polynucleotide having a nucleotide sequence encoding a light chain variable region of an anti-CD137 antibody consisting of an amino acid sequence at amino acid positions 134-244 of SEQ ID NO: 32; or
(i) a polynucleotide having a nucleotide sequence encoding a heavy chain variable region of an anti-CD137 antibody consisting of an amino acid sequence at amino acid positions 1-121 of SEQ ID NO: 34, and a polynucleotide having a nucleotide sequence encoding a light chain variable region of an anti-CD137 antibody consisting of an amino acid sequence at amino acid positions 137-247 of SEQ ID NO: 34.

In one embodiment, the polynucleotide of the bispecific antibody of the present invention is a polynucleotide having a nucleotide sequence encoding an anti-CD137 scFv, which is selected from the group consisting of the following (a) to (e):
(a) a polynucleotide having a nucleotide sequence encoding an anti-CD137 scFv consisting of an amino acid sequence at amino acid positions 1-242 of SEQ ID NO: 26;
(b) a polynucleotide having a nucleotide sequence encoding an anti-CD137 scFv consisting of an amino acid sequence at amino acid positions 1-244 of SEQ ID NO: 28;
(c) a polynucleotide having a nucleotide sequence encoding an anti-CD137 scFv consisting of an amino acid sequence at amino acid positions 1-249 of SEQ ID NO: 30;
(d) a polynucleotide having a nucleotide sequence encoding an anti-CD137 scFv consisting of an amino acid sequence at amino acid positions 1-244 of SEQ ID NO: 32; or
(e) a polynucleotide having a nucleotide sequence encoding an anti-CD137 scFv consisting of an amino acid sequence at amino acid positions 1-247 of SEQ ID NO: 34.

In one embodiment, the polynucleotide of the bispecific antibody of the present invention is a polynucleotide selected from the group consisting of the following (a) to (e):
(a) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv;
(b) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv;
(c) a polynucleotide having a nucleotide sequence encoding a light chain of an anti-CLDN4 antibody consisting of an amino acid sequence at amino acid positions 1-215 of SEQ ID NO: 40;
(d) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40; or
(e) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40.

The polynucleotide of the bispecific antibody of the present invention can be easily produced by those skilled in the art based on the nucleotide sequence by employing a method known in this field. For example, the polynucleotide of the bispecific antibody of the present invention can be synthesized by utilizing a gene synthesis method known in this field. As such a gene synthesis method, various methods known to those skilled in the art such as a synthesis method for an antibody gene described in WO90/07861 can be employed.

### <Expression Vector for Bispecific Antibody of Invention>

The present invention also provides an expression vector containing a polynucleotide of the bispecific antibody of the present invention (also referred to as the “expression vector for the bispecific antibody of the present invention”). These polynucleotides may be contained respectively in different vectors, or a plurality of the polynucleotides may be contained in one vector.

In one embodiment, the expression vector for the bispecific antibody of the present invention contains a polynucleotide selected from the group consisting of the following (a) to (aa):
(a) a polynucleotide having a nucleotide sequence encoding a heavy chain variable region of an anti-CLDN4 antibody consisting of an amino acid sequence at amino acid positions 1-125 of SEQ ID NO: 2;
(b) a polynucleotide having a nucleotide sequence encoding a light chain variable region of an anti-CLDN4 antibody consisting of an amino acid sequence at amino acid positions 1-109 of SEQ ID NO: 4;
(c) a polynucleotide having a nucleotide sequence encoding a heavy chain variable region of an anti-CLDN4 antibody consisting of an amino acid sequence at amino acid positions 1-123 of SEQ ID NO: 6;
(d) a polynucleotide having a nucleotide sequence encoding a light chain variable region of an anti-CLDN4 antibody consisting of an amino acid sequence at amino acid positions 1-109 of SEQ ID NO: 8;
(e) a polynucleotide having a nucleotide sequence encoding a heavy chain variable region of an anti-CD137 antibody consisting of an amino acid sequence at amino acid positions 1-118 of SEQ ID NO: 10;
(f) a polynucleotide having a nucleotide sequence encoding a light chain variable region of an anti-CD137 antibody consisting of an amino acid sequence at amino acid positions 1-109 of SEQ ID NO: 12;
(g) a polynucleotide having a nucleotide sequence encoding a heavy chain variable region of an anti-CD137 antibody consisting of an amino acid sequence at amino acid positions 1-118 of SEQ ID NO: 14;
(h) a polynucleotide having a nucleotide sequence encoding a light chain variable region of an anti-CD137 antibody consisting of an amino acid sequence at amino acid positions 1-111 of SEQ ID NO: 16;
(i) a polynucleotide having a nucleotide sequence encoding a heavy chain variable region of an anti-CD137 antibody consisting of an amino acid sequence at amino acid positions 1-118 of SEQ ID NO: 18;
(j) a polynucleotide having a nucleotide sequence encoding a light chain variable region of an anti-CD137 antibody consisting of an amino acid sequence at amino acid positions 1-111 of SEQ ID NO: 20;
(k) a polynucleotide having a nucleotide sequence encoding a heavy chain variable region of an anti-CD137 antibody consisting of an amino acid sequence at amino acid positions 1-121 of SEQ ID NO: 22;
(l) a polynucleotide having a nucleotide sequence encoding a light chain variable region of an anti-CD137 antibody consisting of an amino acid sequence at amino acid positions 1-111 of SEQ ID NO: 24;
(m) a polynucleotide having a nucleotide sequence encoding a heavy chain variable region of an anti-CD137 antibody consisting of an amino acid sequence at amino acid positions 1-118 of SEQ ID NO: 26;
(n) a polynucleotide having a nucleotide sequence encoding a light chain variable region of an anti-CD137 antibody consisting of an amino acid sequence at amino acid positions 134-242 of SEQ ID NO: 26;
(o) a polynucleotide having a nucleotide sequence encoding a heavy chain variable region of an anti-CD137 antibody consisting of an amino acid sequence at amino acid positions 1-118 of SEQ ID NO: 28;
(p) a polynucleotide having a nucleotide sequence encoding a light chain variable region of an anti-CD137 antibody consisting of an amino acid sequence at amino acid positions 134-244 of SEQ ID NO: 28;
(q) a polynucleotide having a nucleotide sequence encoding a light chain variable region of an anti-CD137 antibody consisting of an amino acid sequence at amino acid positions 1-111 of SEQ ID NO: 30;
(r) a polynucleotide having a nucleotide sequence encoding a heavy chain variable region of an anti-CD137 antibody consisting of an amino acid sequence at amino acid positions 132-249 of SEQ ID NO: 30;
(s) a polynucleotide having a nucleotide sequence encoding a heavy chain variable region of an anti-CD137 antibody consisting of an amino acid sequence at amino acid positions 1-118 of SEQ ID NO: 32;
(t) a polynucleotide having a nucleotide sequence encoding a light chain variable region of an anti-CD137 antibody consisting of an amino acid sequence at amino acid positions 134-244 of SEQ ID NO: 32;
(u) a polynucleotide having a nucleotide sequence encoding a heavy chain variable region of an anti-CD137 antibody consisting of an amino acid sequence at amino acid positions 1-121 of SEQ ID NO: 34;
(v) a polynucleotide having a nucleotide sequence encoding a light chain variable region of an anti-CD137 antibody consisting of an amino acid sequence at amino acid positions 137-247 of SEQ ID NO: 34;
(w) a polynucleotide having a nucleotide sequence encoding an anti-CD137 scFv consisting of an amino acid sequence at amino acid positions 1-242 of SEQ ID NO: 26; (x) a polynucleotide having a nucleotide sequence encoding an anti-CD137 scFv consisting of an amino acid sequence at amino acid positions 1-244 of SEQ ID NO: 28;
(y) a polynucleotide having a nucleotide sequence encoding an anti-CD137 scFv consisting of an amino acid sequence at amino acid positions 1-249 of SEQ ID NO: 30; (z) a polynucleotide having a nucleotide sequence encoding an anti-CD137 scFv consisting of an amino acid sequence at amino acid positions 1-244 of SEQ ID NO: 32; or
(aa) a polynucleotide having a nucleotide sequence encoding an anti-CD137 scFv consisting of an amino acid sequence at amino acid positions 1-247 of SEQ ID NO: 34.

In one embodiment, the expression vector for the bispecific antibody of the present invention contains a polynucleotide selected from the group consisting of the following (a) to (k):
(a) a polynucleotide having a nucleotide sequence encoding a heavy chain variable region of an anti-CLDN4 antibody consisting of an amino acid sequence at amino acid positions 1-125 of SEQ ID NO: 2, and a polynucleotide having a nucleotide sequence encoding a light chain variable region of an anti-CLDN4 antibody consisting of an amino acid sequence at amino acid positions 1-109 of SEQ ID NO: 4;
(b) a polynucleotide having a nucleotide sequence encoding a heavy chain variable region of an anti-CLDN4 antibody consisting of an amino acid sequence at amino acid positions 1-123 of SEQ ID NO: 6, and a polynucleotide having a nucleotide sequence encoding a light chain variable region of an anti-CLDN4 antibody consisting of an amino acid sequence at amino acid positions 1-109 of SEQ ID NO: 8;
(c) a polynucleotide having a nucleotide sequence encoding a heavy chain variable region of an anti-CD137 antibody consisting of an amino acid sequence at amino acid positions 1-118 of SEQ ID NO: 10, and a polynucleotide having a nucleotide sequence encoding a light chain variable region of an anti-CD137 antibody consisting of an amino acid sequence at amino acid positions 1-109 of SEQ ID NO: 12;
(d) a polynucleotide having a nucleotide sequence encoding a heavy chain variable region of an anti-CD137 antibody consisting of an amino acid sequence at amino acid positions 1-118 of SEQ ID NO: 14, and a polynucleotide having a nucleotide sequence encoding a light chain variable region of an anti-CD137 antibody consisting of an amino acid sequence at amino acid positions 1-111 of SEQ ID NO: 16;
(e) a polynucleotide having a nucleotide sequence encoding a heavy chain variable region of an anti-CD137 antibody consisting of an amino acid sequence at amino acid positions 1-118 of SEQ ID NO: 18, and a polynucleotide having a nucleotide sequence encoding a light chain variable region of an anti-CD137 antibody consisting of an amino acid sequence at amino acid positions 1-111 of SEQ ID NO: 20;
(f) a polynucleotide having a nucleotide sequence encoding a heavy chain variable region of an anti-CD137 antibody consisting of an amino acid sequence at amino acid positions 1-121 of SEQ ID NO: 22, and a polynucleotide having a nucleotide sequence encoding a light chain variable region of an anti-CD137 antibody consisting of an amino acid sequence at amino acid positions 1-111 of SEQ ID NO: 24;
(g) a polynucleotide having a nucleotide sequence encoding a heavy chain variable region of an anti-CD137 antibody consisting of an amino acid sequence at amino acid positions 1-118 of SEQ ID NO: 26, and a polynucleotide having a nucleotide sequence encoding a light chain variable region of an anti-CD137 antibody consisting of an amino acid sequence at amino acid positions 134-242 of SEQ ID NO: 26;
(h) a polynucleotide having a nucleotide sequence encoding a heavy chain variable region of an anti-CD137 antibody consisting of an amino acid sequence at amino acid positions 1-118 of SEQ ID NO: 28, and a polynucleotide having a nucleotide sequence encoding a light chain variable region of an anti-CD137 antibody consisting of an amino acid sequence at amino acid positions 134-244 of SEQ ID NO: 28;
(i) a polynucleotide having a nucleotide sequence encoding a light chain variable region of an anti-CD137 antibody consisting of an amino acid sequence at amino acid positions 1-111 of SEQ ID NO: 30, and a polynucleotide having a nucleotide sequence encoding a heavy chain variable region of an anti-CD137 antibody consisting of an amino acid sequence at amino acid positions 132-249 of SEQ ID NO: 30;
(j) a polynucleotide having a nucleotide sequence encoding a heavy chain variable region of an anti-CD137 antibody consisting of an amino acid sequence at amino acid positions 1-118 of SEQ ID NO: 32, and a polynucleotide having a nucleotide sequence encoding a light chain variable region of an anti-CD137 antibody consisting of an amino acid sequence at amino acid positions 134-244 of SEQ ID NO: 32; or
(k) a polynucleotide having a nucleotide sequence encoding a heavy chain variable region of an anti-CD137 antibody consisting of an amino acid sequence at amino acid positions 1-121 of SEQ ID NO: 34, and a polynucleotide having a nucleotide sequence encoding a light chain variable region of an anti-CD137 antibody consisting of an amino acid sequence at amino acid positions 137-247 of SEQ ID NO: 34.

In one embodiment, the expression vector for the bispecific antibody of the present invention contains a polynucleotide selected from the group consisting of the following (a) to (c):
(a) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv;
(b) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv; or
(c) a polynucleotide having a nucleotide sequence encoding a light chain of an anti-CLDN4 antibody consisting of an amino acid sequence at amino acid positions 1-215 of SEQ ID NO: 40.

In one embodiment, the expression vector for the bispecific antibody of the present invention contains a polynucleotide selected from the following (a) or (b):
(a) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40; or
(b) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40.

The expression vector for the bispecific antibody of the present invention is not especially limited as long as it can produce the polynucleotide of the present invention in various host cells such as a eukaryotic cell (such as an animal cell, an insect cell, a plant cell or a yeast) and/or a prokaryotic cell (such as E. coli). Examples of such an expression vector include a plasmid vector and a viral vector. As the plasmid vector, for example, pcDNA series (Thermo Fisher Scientific), pALTER®-MAX (Promega Corporation), pHEK293 Ultra Expression Vector (Takara Bio Inc.), pEE 6.4 or pEE 12.4 (Lonza Biologics) or the like can be used. As the viral vector, for example, a lentivirus, an adenovirus, a retrovirus, or an adeno-associated virus can be used. For example, when a lentivirus is used for introducing the polynucleotide of the present invention into a cell, pLVSIN-CMV/EF1α vector (Takara Bio Inc.), pLenti vector (Thermo Fisher Scientific) or the like can be used as the lentivirus. In one embodiment, a vector used in the expression vector for the bispecific antibody of the present invention is pcDNA 3.4-TOPO® (Thermo Fisher Scientific) or pcDNA 3.1 (Thermo Fisher Scientific).

The expression vector for the bispecific antibody of the present invention can contain a promoter operably linked to the polynucleotide of the bispecific antibody of the present invention. Examples of the promoter for expressing the polynucleotide of the bispecific antibody of the present invention in an animal cell include virus-derived promoters such as CMV, RSV and SV40, an actin promoter, an EF (elongation factor) la promoter, and a heat shock promoter. Examples of the promoter for expressing the polynucleotide of the bispecific antibody of the present invention in a bacterium (such as one belonging to the genus Escherichia) include a trp promoter, a lac promoter, a λPL promoter, and a tac promoter. Examples of the promoter for expressing the polynucleotide of the bispecific antibody of the present invention in a yeast include a GAL1 promoter, a GAL10 promoter, a PH05 promoter, a PGK promoter, a GAP promoter, and an ADH promoter.

When an animal cell, an insect cell or a yeast is used as the host cell, the expression vector for the bispecific antibody of the present invention can contain a start codon and a stop codon. In this case, an enhancer sequence, the 5′-side and 3′-side untranslated regions of a gene encoding the antibody of the present invention, or the heavy chain or light chain thereof, a secretory signal sequence, a splice joint, a polyadenylation site, a replicable unit or the like may be contained. When E. coli is used as the host cell, the expression vector of the present invention can contain a start codon, a stop codon, a terminator region, and a replicable unit. The expression vector of the present invention may contain a drug selection marker gene usually used depending on purpose (such as a tetracycline resistance gene, an ampicillin resistance gene, a kanamycin resistance gene, a neomycin resistance gene, or a dihydrofolate reductase gene).

### <Host Cell of Invention>

The present invention also provides a host cell transformed by the polynucleotide of the bispecific antibody of the present invention or the expression vector for the bispecific antibody of the present invention (also referred to as the “polynucleotide or the like of the present invention”) (also referred to as the “host cell of the present invention”). The host cell of the present invention contains the polynucleotide or the like of the present invention in the cell. The introduced polynucleotide or the like of the present invention may be or may not be integrated into the genomic DNA of the host cell. A cell used as the host cell may be either a cell capable of being cultured in vitro or an in vivo cell. When the host cell is a cell capable of being cultured in vitro, the host cell of the present invention can be produced by introducing the polynucleotide or the like of the present invention into the cell in vitro. A method for transforming the host cell is not especially limited, and for example, a method generally employed by those skilled in the art such as the calcium phosphate method, the electroporation method or the lipofection method can be employed. When the host cell is an in vivo cell, a method for introducing the polynucleotide or the like of the present invention into the host cell is not especially limited, and a nucleic acid delivery carrier (cationic carrier or non-cationic carrier (for example, including, but not limited to, a liposome and a lipid nanoparticle (LNP)) and the like) can be used.

The cell capable of being cultured in vitro is not especially limited as long as it can be transformed by the expression vector to be used or a method such as electroporation to express an antibody or a polypeptide. Examples of the cell capable of being cultured in vitro include various cells including conventional cells usually used in this technical field and artificially established cells (for example, animal cells (such as a CHO-K1 cell, an ExpiCHO-S® cell, a CHOK1SV cell, a CHO-DG44 cell, a HEK293 cell, an Expi293F cell, and a NS0 cell), insect cells (such as Sf9), bacteria (such as those belonging to the genus Escherichia), and yeasts (such as those belonging to the genus Saccharomyces and the genus Pichia)). In one embodiment, the host cell of the present invention is an Expi293F cell, a CHO-K1 cell or an ExpiCHO-S cell.

Selection of a transformed host cell in vitro can be performed by a method generally employed by those skilled in the art. As the selection method, for example, a drug selection method using a drug selection marker gene and a drug such as tetracycline, ampicillin, neomycin or hygromycin, or a cell isolation method such as a limiting dilution method, a single cell sorting method, or a colony pick-up method can be employed.

In one embodiment of the host cell of the present invention, the host cell contains a polynucleotide selected from the group consisting of the following (a) to (aa):
(a) a polynucleotide having a nucleotide sequence encoding a heavy chain variable region of an anti-CLDN4 antibody consisting of an amino acid sequence at amino acid positions 1-125 of SEQ ID NO: 2;
(b) a polynucleotide having a nucleotide sequence encoding a light chain variable region of an anti-CLDN4 antibody consisting of an amino acid sequence at amino acid positions 1-109 of SEQ ID NO: 4;
(c) a polynucleotide having a nucleotide sequence encoding a heavy chain variable region of an anti-CLDN4 antibody consisting of an amino acid sequence at amino acid positions 1-123 of SEQ ID NO: 6;
(d) a polynucleotide having a nucleotide sequence encoding a light chain variable region of an anti-CLDN4 antibody consisting of an amino acid sequence at amino acid positions 1-109 of SEQ ID NO: 8;
(e) a polynucleotide having a nucleotide sequence encoding a heavy chain variable region of an anti-CD137 antibody consisting of an amino acid sequence at amino acid positions 1-118 of SEQ ID NO: 10;
(f) a polynucleotide having a nucleotide sequence encoding a light chain variable region of an anti-CD137 antibody consisting of an amino acid sequence at amino acid positions 1-109 of SEQ ID NO: 12;
(g) a polynucleotide having a nucleotide sequence encoding a heavy chain variable region of an anti-CD137 antibody consisting of an amino acid sequence at amino acid positions 1-118 of SEQ ID NO: 14;
(h) a polynucleotide having a nucleotide sequence encoding a light chain variable region of an anti-CD137 antibody consisting of an amino acid sequence at amino acid positions 1-111 of SEQ ID NO: 16;
(i) a polynucleotide having a nucleotide sequence encoding a heavy chain variable region of an anti-CD137 antibody consisting of an amino acid sequence at amino acid positions 1-118 of SEQ ID NO: 18;
(j) a polynucleotide having a nucleotide sequence encoding a light chain variable region of an anti-CD137 antibody consisting of an amino acid sequence at amino acid positions 1-111 of SEQ ID NO: 20;
(k) a polynucleotide having a nucleotide sequence encoding a heavy chain variable region of an anti-CD137 antibody consisting of an amino acid sequence at amino acid positions 1-121 of SEQ ID NO: 22;
(l) a polynucleotide having a nucleotide sequence encoding a light chain variable region of an anti-CD137 antibody consisting of an amino acid sequence at amino acid positions 1-111 of SEQ ID NO: 24;
(m) a polynucleotide having a nucleotide sequence encoding a heavy chain variable region of an anti-CD137 antibody consisting of an amino acid sequence at amino acid positions 1-118 of SEQ ID NO: 26;
(n) a polynucleotide having a nucleotide sequence encoding a light chain variable region of an anti-CD137 antibody consisting of an amino acid sequence at amino acid positions 134-242 of SEQ ID NO: 26;
(o) a polynucleotide having a nucleotide sequence encoding a heavy chain variable region of an anti-CD137 antibody consisting of an amino acid sequence at amino acid positions 1-118 of SEQ ID NO: 28;
(p) a polynucleotide having a nucleotide sequence encoding a light chain variable region of an anti-CD137 antibody consisting of an amino acid sequence at amino acid positions 134-244 of SEQ ID NO: 28;
(q) a polynucleotide having a nucleotide sequence encoding a light chain variable region of an anti-CD137 antibody consisting of an amino acid sequence at amino acid positions 1-111 of SEQ ID NO: 30;
(r) a polynucleotide having a nucleotide sequence encoding a heavy chain variable region of an anti-CD137 antibody consisting of an amino acid sequence at amino acid positions 132-249 of SEQ ID NO: 30;
(s) a polynucleotide having a nucleotide sequence encoding a heavy chain variable region of an anti-CD137 antibody consisting of an amino acid sequence at amino acid positions 1-118 of SEQ ID NO: 32;
(t) a polynucleotide having a nucleotide sequence encoding a light chain variable region of an anti-CD137 antibody consisting of an amino acid sequence at amino acid positions 134-244 of SEQ ID NO: 32;
(u) a polynucleotide having a nucleotide sequence encoding a heavy chain variable region of an anti-CD137 antibody consisting of an amino acid sequence at amino acid positions 1-121 of SEQ ID NO: 34;
(v) a polynucleotide having a nucleotide sequence encoding a light chain variable region of an anti-CD137 antibody consisting of an amino acid sequence at amino acid positions 137-247 of SEQ ID NO: 34;
(w) a polynucleotide having a nucleotide sequence encoding an anti-CD137 scFv consisting of an amino acid sequence at amino acid positions 1-242 of SEQ ID NO: 26;
(x) a polynucleotide having a nucleotide sequence encoding an anti-CD137 scFv consisting of an amino acid sequence at amino acid positions 1-244 of SEQ ID NO: 28;
(y) a polynucleotide having a nucleotide sequence encoding an anti-CD137 scFv consisting of an amino acid sequence at amino acid positions 1-249 of SEQ ID NO: 30;
(z) a polynucleotide having a nucleotide sequence encoding an anti-CD137 scFv consisting of an amino acid sequence at amino acid positions 1-244 of SEQ ID NO: 32; or (aa) a polynucleotide having a nucleotide sequence encoding an anti-CD137 scFv consisting of an amino acid sequence at amino acid positions 1-247 of SEQ ID NO: 34.

In one embodiment, the host cell contains a polynucleotide selected from the group consisting of the following (a) or (k):
(a) a polynucleotide having a nucleotide sequence encoding a heavy chain variable region of an anti-CLDN4 antibody consisting of an amino acid sequence at amino acid positions 1-125 of SEQ ID NO: 2, and a polynucleotide having a nucleotide sequence encoding a light chain variable region of an anti-CLDN4 antibody consisting of an amino acid sequence at amino acid positions 1-109 of SEQ ID NO: 4;
(b) a polynucleotide having a nucleotide sequence encoding a heavy chain variable region of an anti-CLDN4 antibody consisting of an amino acid sequence at amino acid positions 1-123 of SEQ ID NO: 6, and a polynucleotide having a nucleotide sequence encoding a light chain variable region of an anti-CLDN4 antibody consisting of an amino acid sequence at amino acid positions 1-109 of SEQ ID NO: 8;
(c) a polynucleotide having a nucleotide sequence encoding a heavy chain variable region of an anti-CD137 antibody consisting of an amino acid sequence at amino acid positions 1-118 of SEQ ID NO: 10, and a polynucleotide having a nucleotide sequence encoding a light chain variable region of an anti-CD137 antibody consisting of an amino acid sequence at amino acid positions 1-109 of SEQ ID NO: 12;
(d) a polynucleotide having a nucleotide sequence encoding a heavy chain variable region of an anti-CD137 antibody consisting of an amino acid sequence at amino acid positions 1-118 of SEQ ID NO: 14, and a polynucleotide having a nucleotide sequence encoding a light chain variable region of an anti-CD137 antibody consisting of an amino acid sequence at amino acid positions 1-111 of SEQ ID NO: 16;
(e) a polynucleotide having a nucleotide sequence encoding a heavy chain variable region of an anti-CD137 antibody consisting of an amino acid sequence at amino acid positions 1-118 of SEQ ID NO: 18, and a polynucleotide having a nucleotide sequence encoding a light chain variable region of an anti-CD137 antibody consisting of an amino acid sequence at amino acid positions 1-111 of SEQ ID NO: 20;
(f) a polynucleotide having a nucleotide sequence encoding a heavy chain variable region of an anti-CD137 antibody consisting of an amino acid sequence at amino acid positions 1-121 of SEQ ID NO: 22, and a polynucleotide having a nucleotide sequence encoding a light chain variable region of an anti-CD137 antibody consisting of an amino acid sequence at amino acid positions 1-111 of SEQ ID NO: 24;
(g) a polynucleotide having a nucleotide sequence encoding a heavy chain variable region of an anti-CD137 antibody consisting of an amino acid sequence at amino acid positions 1-118 of SEQ ID NO: 26, and a polynucleotide having a nucleotide sequence encoding a light chain variable region of an anti-CD137 antibody consisting of an amino acid sequence at amino acid positions 134-242 of SEQ ID NO: 26;
(h) a polynucleotide having a nucleotide sequence encoding a heavy chain variable region of an anti-CD137 antibody consisting of an amino acid sequence at amino acid positions 1-118 of SEQ ID NO: 28, and a polynucleotide having a nucleotide sequence encoding a light chain variable region of an anti-CD137 antibody consisting of an amino acid sequence at amino acid positions 134-244 of SEQ ID NO: 28;
(i) a polynucleotide having a nucleotide sequence encoding a light chain variable region of an anti-CD137 antibody consisting of an amino acid sequence at amino acid positions 1-111 of SEQ ID NO: 30, and a polynucleotide having a nucleotide sequence encoding a heavy chain variable region of an anti-CD137 antibody consisting of an amino acid sequence at amino acid positions 132-249 of SEQ ID NO: 30;
(j) a polynucleotide having a nucleotide sequence encoding a heavy chain variable region of an anti-CD137 antibody consisting of an amino acid sequence at amino acid positions 1-118 of SEQ ID NO: 32, and a polynucleotide having a nucleotide sequence encoding a light chain variable region of an anti-CD137 antibody consisting of an amino acid sequence at amino acid positions 134-244 of SEQ ID NO: 32; or
(k) a polynucleotide having a nucleotide sequence encoding a heavy chain variable region of an anti-CD137 antibody consisting of an amino acid sequence at amino acid positions 1-121 of SEQ ID NO: 34, and a polynucleotide having a nucleotide sequence encoding a light chain variable region of an anti-CD137 antibody consisting of an amino acid sequence at amino acid positions 137-247 of SEQ ID NO: 34.

In one embodiment of the host cell of the present invention, the host cell contains a polynucleotide selected from the group consisting of the following (a) to (c):
(a) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv;
(b) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv; or
(c) a polynucleotide having a nucleotide sequence encoding a light chain of an anti-CLDN4 antibody consisting of an amino acid sequence at amino acid positions 1-215 of SEQ ID NO: 40.

In one embodiment, the host cell contains a polynucleotide selected from the following (a) or (b):
(a) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40; or
(b) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40.

In one embodiment of the host cell of the present invention, the host cell contains polypeptides described in the following (a) to (j):
(a) a polynucleotide having a nucleotide sequence encoding a polypeptide containing a heavy chain of an anti-CLDN4 antibody containing a heavy chain variable region consisting of an amino acid sequence at amino acid positions 1-125 of SEQ ID NO: 2, and an anti-CD137 scFv containing a heavy chain variable region of an anti-CD137 antibody consisting of an amino acid sequence at amino acid positions 1-118 of SEQ ID NO: 26 and a light chain variable region of an anti-CD137 antibody consisting of an amino acid sequence at amino acid positions 134-242 of SEQ ID NO: 26, in which the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 4;
(b) a polynucleotide having a nucleotide sequence encoding a polypeptide containing a heavy chain of an anti-CLDN4 antibody containing a heavy chain variable region consisting of an amino acid sequence at amino acid positions 1-125 of SEQ ID NO: 2, and an anti-CD137 scFv containing a heavy chain variable region of an anti-CD137 antibody consisting of an amino acid sequence at amino acid positions 1-118 of SEQ ID NO: 28 and a light chain variable region of an anti-CD137 antibody consisting of an amino acid sequence at amino acid positions 134-244 of SEQ ID NO: 28, in which the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 4;
(c) a polynucleotide having a nucleotide sequence encoding a polypeptide containing a heavy chain of an anti-CLDN4 antibody containing a heavy chain variable region consisting of an amino acid sequence at amino acid positions 1-125 of SEQ ID NO: 2, and an anti-CD137 scFv containing a light chain variable region of an anti-CD137 antibody consisting of an amino acid sequence at amino acid positions 1-111 of SEQ ID NO: 30 and a heavy chain variable region of an anti-CD137 antibody consisting of an amino acid sequence at amino acid positions 132-249 of SEQ ID NO: 30, in which the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 4;
(d) a polynucleotide having a nucleotide sequence encoding a polypeptide containing a heavy chain of an anti-CLDN4 antibody containing a heavy chain variable region consisting of an amino acid sequence at amino acid positions 1-125 of SEQ ID NO: 2, and an anti-CD137 scFv containing a heavy chain variable region of an anti-CD137 antibody consisting of an amino acid sequence at amino acid positions 1-118 of SEQ ID NO: 32 and a light chain variable region of an anti-CD137 antibody consisting of an amino acid sequence at amino acid positions 134-244 of SEQ ID NO: 32, in which the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 4;
(e) a polynucleotide having a nucleotide sequence encoding a polypeptide, which contains a heavy chain of an anti-CLDN4 antibody containing a heavy chain variable region consisting of an amino acid sequence at amino acid positions 1-125 of SEQ ID NO: 2, and an anti-CD137 scFv containing a heavy chain variable region of an anti-CD137 antibody consisting of an amino acid sequence at amino acid positions 1-121 of SEQ ID NO: 34 and a light chain variable region of an anti-CD137 antibody consisting of an amino acid sequence at amino acid positions 137-247 of SEQ ID NO: 34, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 4;
(f) a polynucleotide having a nucleotide sequence encoding a polypeptide containing a heavy chain of an anti-CLDN4 antibody containing a heavy chain variable region consisting of an amino acid sequence at amino acid positions 1-123 of SEQ ID NO: 6, and an anti-CD137 scFv containing a heavy chain variable region of an anti-CD137 antibody consisting of an amino acid sequence at amino acid positions 1-118 of SEQ ID NO: 26 and a light chain variable region of an anti-CD137 antibody consisting of an amino acid sequence at amino acid positions 134-242 of SEQ ID NO: 26, in which the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 8;
(g) a polynucleotide having a nucleotide sequence encoding a polypeptide containing a heavy chain of an anti-CLDN4 antibody containing a heavy chain variable region consisting of an amino acid sequence at amino acid positions 1-123 of SEQ ID NO: 6, and an anti-CD137 scFv containing a heavy chain variable region of an anti-CD137 antibody consisting of an amino acid sequence at amino acid positions 1-118 of SEQ ID NO: 28 and a light chain variable region of an anti-CD137 antibody consisting of an amino acid sequence at amino acid positions 134-244 of SEQ ID NO: 28, in which the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 8;
(h) a polynucleotide having a nucleotide sequence encoding a polypeptide containing a heavy chain of an anti-CLDN4 antibody containing a heavy chain variable region consisting of an amino acid sequence at amino acid positions 1-123 of SEQ ID NO: 6, and an anti-CD137 scFv containing a light chain variable region of an anti-CD137 antibody consisting of an amino acid sequence at amino acid positions 1-111 of SEQ ID NO: 30 and a heavy chain variable region of an anti-CD137 antibody consisting of an amino acid sequence at amino acid positions 132-249 of SEQ ID NO: 30, in which the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 8;
(i) a polynucleotide having a nucleotide sequence encoding a polypeptide containing a heavy chain of an anti-CLDN4 antibody containing a heavy chain variable region consisting of an amino acid sequence at amino acid positions 1-123 of SEQ ID NO: 6, and an anti-CD137 scFv containing a heavy chain variable region of an anti-CD137 antibody consisting of an amino acid sequence at amino acid positions 1-118 of SEQ ID NO: 32 and a light chain variable region of an anti-CD137 antibody consisting of an amino acid sequence at amino acid positions 134-244 of SEQ ID NO: 32, in which the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 8; or
(j) a polynucleotide having a nucleotide sequence encoding a polypeptide containing a heavy chain of an anti-CLDN4 antibody containing a heavy chain variable region consisting of an amino acid sequence at amino acid positions 1-123 of SEQ ID NO: 6, and an anti-CD137 scFv containing a heavy chain variable region of an anti-CD137 antibody consisting of an amino acid sequence at amino acid positions 1-121 of SEQ ID NO: 34 and a light chain variable region of an anti-CD137 antibody consisting of an amino acid sequence at amino acid positions 137-247 of SEQ ID NO: 34, in which the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 8.

In one embodiment of the host cell of the present invention, the host cell is a host cell described in the following (a) or (b):
(a) a host cell containing a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40; or
(b) a host cell containing a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40.

In one embodiment of the host cell of the present invention, the host cell is a host cell containing a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40.

### <Method for Producing Bispecific Antibody of Invention>

The present invention also provides a method for producing the anti-CLDN4/anti-CD137 bispecific antibody of the present invention (also referred to as the “production method of the present invention”). The production method of the present invention can include a method for producing the polynucleotide of the bispecific antibody of the present invention, a method for producing the expression vector for the bispecific antibody of the present invention, and a method for producing the host cell of the present invention, described above. Besides, the production method of the present invention can include a step of culturing the host cell described above in <Host Cell of Invention> to express the antibody in the cell or the culture supernatant, a method for collecting, isolating and purifying the antibody, and the like. The production method of the present invention is, however, not limited to these methods as long as the anti-CLDN4/anti-CD137 bispecific antibody of the present invention is produced.

In one embodiment, the production method of the present invention includes a step of culturing a host cell described in the following (a) to (j):
(a) a host cell containing: a polynucleotide having a nucleotide sequence encoding a polypeptide, which contains a heavy chain of an anti-CLDN4 antibody containing a heavy chain variable region consisting of an amino acid sequence at amino acid positions 1-125 of SEQ ID NO: 2, and an anti-CD137 scFv containing a heavy chain variable region of an anti-CD137 antibody consisting of an amino acid sequence at amino acid positions 1-118 of SEQ ID NO: 26 and a light chain variable region of an anti-CD137 antibody consisting of an amino acid sequence at amino acid positions 134-242 of SEQ ID NO: 26, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 4;
(b) a host cell containing: a polynucleotide having a nucleotide sequence encoding a polypeptide, which contains a heavy chain of an anti-CLDN4 antibody containing a heavy chain variable region consisting of an amino acid sequence at amino acid positions 1-125 of SEQ ID NO: 2, and an anti-CD137 scFv containing a heavy chain variable region of an anti-CD137 antibody consisting of an amino acid sequence at amino acid positions 1-118 of SEQ ID NO: 28 and a light chain variable region of an anti-CD137 antibody consisting of an amino acid sequence at amino acid positions 134-244 of SEQ ID NO: 28, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 4;
(c) a host cell containing: a polynucleotide having a nucleotide sequence encoding a polypeptide, which contains a heavy chain of an anti-CLDN4 antibody containing a heavy chain variable region consisting of an amino acid sequence at amino acid positions 1-125 of SEQ ID NO: 2, and an anti-CD137 scFv containing a light chain variable region of an anti-CD137 antibody consisting of an amino acid sequence at amino acid positions 1-111 of SEQ ID NO: 30 and a heavy chain variable region of an anti-CD137 antibody consisting of an amino acid sequence at amino acid positions 132-249 of SEQ ID NO: 30, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 4;
(d) a host cell containing: a polynucleotide having a nucleotide sequence encoding a polypeptide, which contains a heavy chain of an anti-CLDN4 antibody containing a heavy chain variable region consisting of an amino acid sequence at amino acid positions 1-125 of SEQ ID NO: 2, and an anti-CD137 scFv containing a heavy chain variable region of an anti-CD137 antibody consisting of an amino acid sequence at amino acid positions 1-118 of SEQ ID NO: 32 and a light chain variable region of an anti-CD137 antibody consisting of an amino acid sequence at amino acid positions 134-244 of SEQ ID NO: 32, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 4;
(e) a host cell containing: a polynucleotide having a nucleotide sequence encoding a polypeptide, which contains a heavy chain of an anti-CLDN4 antibody containing a heavy chain variable region consisting of an amino acid sequence at amino acid positions 1-125 of SEQ ID NO: 2, and an anti-CD137 scFv containing a heavy chain variable region of an anti-CD137 antibody consisting of an amino acid sequence at amino acid positions 1-121 of SEQ ID NO: 34 and a light chain variable region of an anti-CD137 antibody consisting of an amino acid sequence at amino acid positions 137-247 of SEQ ID NO: 34, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 4;
(f) a host cell containing: a polynucleotide having a nucleotide sequence encoding a polypeptide, which contains a heavy chain of an anti-CLDN4 antibody containing a heavy chain variable region consisting of an amino acid sequence at amino acid positions 1-123 of SEQ ID NO: 6, and an anti-CD137 scFv containing a heavy chain variable region of an anti-CD137 antibody consisting of an amino acid sequence at amino acid positions 1-118 of SEQ ID NO: 26 and a light chain variable region of an anti-CD137 antibody consisting of an amino acid sequence at amino acid positions 134-242 of SEQ ID NO: 26, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 8;
(g) a host cell containing: a polynucleotide having a nucleotide sequence encoding a polypeptide, which contains a heavy chain of an anti-CLDN4 antibody containing a heavy chain variable region consisting of an amino acid sequence at amino acid positions 1-123 of SEQ ID NO: 6, and an anti-CD137 scFv containing a heavy chain variable region of an anti-CD137 antibody consisting of an amino acid sequence at amino acid positions 1-118 of SEQ ID NO: 28 and a light chain variable region of an anti-CD137 antibody consisting of an amino acid sequence at amino acid positions 134-244 of SEQ ID NO: 28, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 8;
(h) a host cell containing: a polynucleotide having a nucleotide sequence encoding a polypeptide, which contains a heavy chain of an anti-CLDN4 antibody containing a heavy chain variable region consisting of an amino acid sequence at amino acid positions 1-123 of SEQ ID NO: 6, and an anti-CD137 scFv containing a light chain variable region of an anti-CD137 antibody consisting of an amino acid sequence at amino acid positions 1-111 of SEQ ID NO: 30 and a heavy chain variable region of an anti-CD137 antibody consisting of an amino acid sequence at amino acid positions 132-249 of SEQ ID NO: 30, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 8;

(i) a host cell containing: a polynucleotide having a nucleotide sequence encoding a polypeptide, which contains a heavy chain of an anti-CLDN4 antibody containing a heavy chain variable region consisting of an amino acid sequence at amino acid positions 1-123 of SEQ ID NO: 6, and an anti-CD137 scFv containing a heavy chain variable region of an anti-CD137 antibody consisting of an amino acid sequence at amino acid positions 1-118 of SEQ ID NO: 32 and a light chain variable region of an anti-CD137 antibody consisting of an amino acid sequence at amino acid positions 134-244 of SEQ ID NO: 32, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 8; or

(j) a host cell containing: a polynucleotide having a nucleotide sequence encoding a polypeptide, which contains a heavy chain of an anti-CLDN4 antibody containing a heavy chain variable region consisting of an amino acid sequence at amino acid positions 1-123 of SEQ ID NO: 6, and an anti-CD137 scFv containing a heavy chain variable region of an anti-CD137 antibody consisting of an amino acid sequence at amino acid positions 1-121 of SEQ ID NO: 34 and a light chain variable region of an anti-CD137 antibody consisting of an amino acid sequence at amino acid positions 137-247 of SEQ ID NO: 34, wherein the amino terminal of the anti-CD137 scFv is linked to the heavy chain carboxy terminal of the anti-CLDN4 antibody via a linker; and a polynucleotide having a nucleotide sequence encoding a light chain of an anti-CLDN4 antibody containing a light chain variable region consisting of an amino acid sequence at amino acid positions 1-109 of SEQ ID NO: 8.

In one embodiment, the production method of the present invention includes a step of culturing a host cell described in the following (a) or (b):
(a) a host cell containing a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40; or
(b) a host cell containing a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40.

The host cell of the present invention can be cultured by a known method. Culture conditions such as a temperature, pH of a medium and a culture time can be appropriately selected by those skilled in the art. When the host cell is an animal cell, for example, MEM medium (Science, 1959, Vol. 130, p. 432-437) containing about 5 to 20% of fetal bovine serum, D-MEM medium (Virol., 1959, Vol. 8, p. 396), RPMI-1640 medium (J. Am. Med. Assoc., 1967, Vol. 199, p. 519), 199 medium (Exp. Biol. Med., 1950, Vol. 73, p. 1-8) or the like can be used as the medium. The pH of the medium is, for example, about 6 to 8, and the culture is performed usually at about 30 to 40° C. for about 15 to 336 hours with aeration or stirring if necessary. When the host cell is an insect cell, for example, Grace's medium (Proc. Natl. Acad. Sci. USA., 1985, Vol. 82, p. 8404) containing fetal bovine serum or the like can be used as the medium. The pH of the medium is, for example, about 5 to 8, and the culture is performed usually at about 20 to 40° C. for about 15 to 100 hours with aeration or stirring if necessary. When the host cell is E. coli or a yeast, for example, a liquid medium containing a nutrition source is suitably used as the medium. A nutrient medium contains, for example, a carbon source, an inorganic nitrogen source, or an organic nitrogen source necessary for growth of the transformed host cell. Examples of the carbon source include glucose, dextran, soluble starch, and sucrose, examples of the inorganic nitrogen source or organic nitrogen source include ammonium salts, nitric acid salts, amino acids, a corn steep liquor, peptone, casein, a meat extract, a soybean cake, and a potato extract. Other nutrients (for example, inorganic salts (such as calcium chloride, sodium dihydrogen phosphate, and magnesium chloride) or vitamins), an antibiotic (such as tetracycline, neomycin, ampicillin, or kanamycin), or the like may be contained if desired. The pH of the medium is, for example, about 5 to 8. When the host cell is E. coli, for example, LB medium, M9 medium (Molecular Cloning, Cold Spring Harbor Laboratory, Vol. 3, A2.2) or the like can be used as the medium. The culture is performed usually at about 14 to 43° C. for about 3 to 24 hours with aeration or stirring if necessary. When the host cell is a yeast, for example, Burkholder minimum medium (Proc. Natl. Acad. Sci. USA., 1980, Vol. 77, p. 4505) or the like can be used as the medium. The culture is performed usually at about 20 to 35° C. for about 14 to 144 hours with aeration or stirring if necessary. Through such culture, the anti-CLDN4/anti-CD137 bispecific antibody of the present invention can be expressed.

The production method of the present invention can include, in addition to the step of culturing the host cell of the present invention to express the anti-CLDN4/anti-CD137 bispecific antibody, a step of collecting, isolating or purifying the anti-CLDN4/anti-CD137 bispecific antibody from the host cell. Examples of an isolating or purifying method include a method utilizing solubility such as salting-out or a solvent precipitation method, a method utilizing a difference in a molecular weight such as dialysis, ultrafiltration and gel filtration, a method utilizing charge such as ion exchange chromatography or hydroxyapatite chromatography, a method utilizing specific affinity such as affinity chromatography, a method utilizing a difference in hydrophobicity such as reverse phase high performance liquid chromatography, and a method utilizing a difference in an isoelectric point such as isoelectric focusing. In one embodiment, the antibody secreted into a culture supernatant can be purified by various chromatographies such as column chromatography using a protein A column or a protein G column.

The anti-CLDN4/anti-CD137 bispecific antibody of the present invention embraces an anti-CLDN4/anti-CD137 bispecific antibody and an anti-CLDN4/anti-CD137 scFv bispecific antibody produced by the production method of the present invention.

### <Pharmaceutical Composition or the Like of Invention>

The present invention also provides a pharmaceutical composition or the like containing the anti-CLDN4/anti-CD137 bispecific antibody of the present invention (also referred to as a “pharmaceutical composition of the present invention”). The pharmaceutical composition of the present invention embraces a pharmaceutical composition containing the anti-CLDN4/anti-CD137 bispecific antibody of the present invention and a pharmaceutically acceptable excipient. The pharmaceutical composition of the present invention can be prepared by a usually employed method with an excipient usually used in this field, namely, a pharmaceutical excipient, a pharmaceutical carrier or the like. Examples of a dosage form of such a pharmaceutical composition include parenteral agents such as an injection and a drop, and administration can be performed by an appropriate method such as intravenous administration, subcutaneous administration, intraperitoneal administration, or intratumoral administration. In formulation, an excipient, a carrier, an additive or the like suitable to the dosage form can be used in a pharmaceutically acceptable range.

The pharmaceutical composition of the present invention can contain a post-translationally modified product of the anti-CLDN4/anti-CD137 bispecific antibody of the present invention. For example, a pharmaceutical composition containing an antibody or the like containing both of or one of lysine deletion in the C-terminal and pyroglutamylation in the N-terminal can be embraced in the present invention.

In one embodiment, the pharmaceutical composition of the present invention is a pharmaceutical composition containing the anti-CLDN4/anti-CD137 bispecific antibody of the present invention and/or a post-translationally modified product of the antibody described in any of the following (a) to (j):
(a) an anti-CLDN4/anti-CD137 bispecific antibody and/or a post-translationally modified product of the antibody containing a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26, in which the amino terminal of the anti-CD137 scFv is linked to the heavy chain carboxy terminal of the anti-CLDN4 antibody via a linker;
(b) an anti-CLDN4/anti-CD137 bispecific antibody and/or a post-translationally modified product of the antibody containing a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28, in which the amino terminal of the anti-CD137 scFv is linked to the heavy chain carboxy terminal of the anti-CLDN4 antibody via a linker;
(c) an anti-CLDN4/anti-CD137 bispecific antibody and/or a post-translationally modified product of the antibody containing a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30, in which the amino terminal of the anti-CD137 scFv is linked to the heavy chain carboxy terminal of the anti-CLDN4 antibody via a linker;
(d) an anti-CLDN4/anti-CD137 bispecific antibody and/or a post-translationally modified product of the antibody containing a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32, in which the amino terminal of the anti-CD137 scFv is linked to the heavy chain carboxy terminal of the anti-CLDN4 antibody via a linker;
(e) an anti-CLDN4/anti-CD137 bispecific antibody and/or a post-translationally modified product of the antibody containing a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34, in which the amino terminal of the anti-CD137 scFv is linked to the heavy chain carboxy terminal of the anti-CLDN4 antibody via a linker;
(f) an anti-CLDN4/anti-CD137 bispecific antibody and/or a post-translationally modified product of the antibody containing a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26, in which the amino terminal of the anti-CD137 scFv is linked to the heavy chain carboxy terminal of the anti-CLDN4 antibody via a linker;
(g) an anti-CLDN4/anti-CD137 bispecific antibody and/or a post-translationally modified product of the antibody containing a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28, in which the amino terminal of the anti-CD137 scFv is linked to the heavy chain carboxy terminal of the anti-CLDN4 antibody via a linker;
(h) an anti-CLDN4/anti-CD137 bispecific antibody and/or a post-translationally modified product of the antibody containing a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30, in which the amino terminal of the anti-CD137 scFv is linked to the heavy chain carboxy terminal of the anti-CLDN4 antibody via a linker;
(i) an anti-CLDN4/anti-CD137 bispecific antibody and/or a post-translationally modified product of the antibody containing a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32, in which the amino terminal of the anti-CD137 scFv is linked to the heavy chain carboxy terminal of the anti-CLDN4 antibody via a linker; or
(j) an anti-CLDN4/anti-CD137 bispecific antibodies and/or a post-translationally modified product of the antibodies containing a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34, in which the amino terminal of the anti-CD137 scFv is linked to the heavy chain carboxy terminal of the anti-CLDN4 antibody via a linker.

In one embodiment, the pharmaceutical composition of the present invention is a pharmaceutical composition containing the anti-CLDN4/anti-CD137 bispecific antibody of the present invention and/or a post-translationally modified product of the antibody described in the following (a) or (b):
(a) an anti-CLDN4/anti-CD137 bispecific antibody and/or a post-translationally modified product of the antibody containing a polypeptide consisting of an amino acid sequence at amino acid positions 1-705 of SEQ ID NO: 36 containing a heavy chain of an anti-CLDN4 antibody and an anti-CD137 scFv, and a light chain of an anti-CLDN4 antibody consisting of an amino acid sequence at amino acid positions 1-215 of SEQ ID NO: 40; or
(b) an anti-CLDN4/anti-CD137 bispecific antibody and/or a post-translationally modified product of the antibody containing a polypeptide consisting of an amino acid sequence at amino acid positions 1-712 of SEQ ID NO: 38 containing a heavy chain of an anti-CLDN4 antibody and an anti-CD137 scFv, and a light chain of an anti-CLDN4 antibody consisting of an amino acid sequence at amino acid positions 1-215 of SEQ ID NO: 40.

The amounts of the anti-CLDN4/anti-CD137 bispecific antibody of the present invention and/or the post-translationally modified product of the antibody added in formulation are varied depending on the degree of symptom and the age of a patient, a dosage form of the formulation to be used, a binding titer of the antibody, or the like, and they can be used in an amount of, for example, about 0.001 mg/kg to 100 mg/kg.

### <Pharmaceutical Use of Anti-CLDN4/anti-CD137 Bispecific Antibody of Invention>

The anti-CLDN4/anti-CD137 bispecific antibody of the present invention and a pharmaceutical composition containing the same can be used for treating cancer of a subject. Besides, the present invention embraces a method for treating cancer including a step of administering a therapeutically effective amount of the anti-CLDN4/anti-CD137 bispecific antibody of the present invention to a subject. Furthermore, the present invention embraces the anti-CLDN4/anti-CD137 bispecific antibody of the present invention for use in treatment of cancer. In addition, the present invention embraces use, in production of a pharmaceutical composition for treating cancer, of the anti-CLDN4/anti-CD137 bispecific antibody of the present invention. Cancer to be treated by the present invention is not especially limited, and examples thereof include various peritoneal metastatic cancers, gastric cancer, lung cancer, blood cancers such as acute lymphoblastic leukemia, acute myelogenous leukemia, Hodgkin's lymphoma, non-Hodgkin's lymphoma, B cell lymphoma, multiple myeloma and T cell lymphoma, solid cancers such as myelodysplastic syndromes, adenocarcinoma, squamous cell carcinoma, adenosquamous carcinoma, undifferentiated carcinoma, large cell carcinoma, non-small cell lung cancer, small cell lung cancer, mesothelioma, skin cancer, skin T cell lymphoma, breast cancer, prostate cancer, bladder cancer, vaginal cancer, cervix cancer, head and neck cancer, uterine cancer, cervical cancer, liver cancer, gallbladder cancer, bile duct cancer, kidney cancer, pancreatic cancer, colon cancer, colorectal cancer, rectal cancer, small intestine cancer, gastric cancer, esophageal cancer, testicular cancer, ovarian cancer and brain tumor, cancers of bone tissues, cartilage tissues, adipose tissues, muscle tissues, vascular tissues and blood-forming tissues, sarcomas such as chondrosarcoma, Ewing's sarcoma, malignant hemangioendothelioma, malignant schwannoma, osteosarcoma and soft tissue sarcoma, and blastomas such as glioblastoma, glioblastoma multiforme, hepatoblastoma, medulloblastoma, nephroblastoma, neuroblastoma, pancreatoblastoma, pleuropulmonary blastoma and retinoblastoma. In one embodiment, cancer to be treated by the present invention is colorectal cancer, non-small cell lung cancer, small cell lung cancer, bladder cancer, ovarian cancer, breast cancer or prostate cancer. In one embodiment, cancer to be treated by the present invention is cancer in which CLDN4 is highly expressed as compared to a normal tissue. Cancer to be treated by the present invention is preferably cancer in which CLDN4 is highly expressed as compared to a normal tissue, or cancer selected from the group consisting of colorectal cancer, rectal cancer, lung cancer, non-small cell lung cancer, small cell lung cancer, bladder cancer, ovarian cancer, breast cancer and prostate cancer.

### <Anti-CLDN4 Antibody of Invention>

The present invention also provides an anti-CLDN4 antibody or its antigen binding fragment described in any of the following (a) to (c):

(a) an anti-CLDN4 antibody or its antigen binding fragment containing a heavy chain variable region containing a CDR1 consisting of an amino acid sequence at amino acid positions 31-35 of SEQ ID NO: 2, a CDR2 consisting of an amino acid sequence at amino acid positions 50-66 of SEQ ID NO: 2, and a CDR3 consisting of an amino acid sequence at amino acid positions 99-114 of SEQ ID NO: 2, and a light chain variable region containing a CDR1 consisting of an amino acid sequence at amino acid positions 24-35 of SEQ ID NO: 4, a CDR2 consisting of an amino acid sequence at amino acid positions 51-57 of SEQ ID NO: 4, and a CDR3 consisting of an amino acid sequence at amino acid positions 90-98 of SEQ ID NO: 4;

(b) an anti-CLDN4 antibody or its antigen binding fragment containing a heavy chain variable region consisting of an amino acid sequence at amino acid positions 1-125 of SEQ ID NO: 2 and a light chain variable region consisting of an amino acid sequence at amino acid positions 1-109 of SEQ ID NO: 4; or

(c) an anti-CLDN4 antibody or its antigen binding fragment containing a heavy chain variable region consisting of an amino acid sequence at amino acid positions 1-123 of SEQ ID NO: 6 and a light chain variable region consisting of an amino acid sequence at amino acid positions 1-109 of SEQ ID NO: 8.

In one embodiment, the anti-CLDN4 antibody of the present invention or its antigen binding fragment is an anti-CLDN4 antibody or its antigen binding fragment containing a heavy chain variable region consisting of an amino acid sequence at amino acid positions 1-125 of SEQ ID NO: 2 and a light chain variable region consisting of an amino acid sequence at amino acid positions 1-109 of SEQ ID NO: 4, or an anti-CLDN4 antibody or its antigen binding fragment containing a heavy chain variable region consisting of an amino acid sequence at amino acid positions 1-123 of SEQ ID NO: 6 and a light chain variable region consisting of an amino acid sequence at amino acid positions 1-109 of SEQ ID NO: 8. In one embodiment, the anti-CLDN4 antibody of the present invention or its antigen binding fragment is a heavy chain variable region consisting of an amino acid sequence at amino acid positions 1-125 of SEQ ID NO: 2. In one embodiment, the anti-CLDN4 antibody of the present invention or its antigen binding fragment is an anti-CLDN4 antibody or its antigen binding fragment containing a heavy chain variable region consisting of an amino acid sequence at amino acid positions 1-123 of SEQ ID NO: 6 and a light chain variable region consisting of an amino acid sequence at amino acid positions 1-109 of SEQ ID NO: 8.

As the heavy chain constant region of the anti-CLDN4 antibody, any of the constant regions Igγ, Igμ, Igα, Igδ and Igε can be selected. Igγ can be selected from, for example, Igγ1, Igγ2, Igγ3 and Igγ4. In one embodiment, the heavy chain constant region is an Igγ1 constant region, and for example, a human Igγ1 constant region. The heavy chain constant region of the anti-CLDN4 antibody of the present invention may contain an amino acid mutation such as LALA mutation, or P331G or P331S mutation in order to deteriorate ADCC and CDC. As the light chain constant region of the anti-CLDN4 antibody of the present invention, either of the constant regions Igλ and Igκ can be selected. In one embodiment, the light chain constant region is an Igκ constant region, and for example, a human Igκ constant region.

In one embodiment, the antigen binding fragment of the anti-CLDN4 antibody of the present invention is a scFv, a Fab, a Fab′, or a F(ab′)_{2.}

In one embodiment, the anti-CLDN4 antibody of the present invention is an anti-CLDN4 antibody containing a heavy chain containing a heavy chain variable region consisting of an amino acid sequence of SEQ ID NO: 2 and a light chain containing a light chain variable region consisting of an amino acid sequence of SEQ ID NO: 4, or an anti-CLDN4 antibody containing a heavy chain containing a heavy chain variable region consisting of an amino acid sequence of SEQ ID NO: 6 and a light chain containing a light chain variable region consisting of an amino acid sequence of SEQ ID NO: 8. In one embodiment, the anti-CLDN4 antibody of the present invention is an anti-CLDN4 antibody containing a heavy chain containing a heavy chain variable region consisting of an amino acid sequence of SEQ ID NO: 2 and a light chain containing a light chain variable region consisting of an amino acid sequence of SEQ ID NO: 4. In one embodiment, the anti-CLDN4 antibody of the present invention is an anti-CLDN4 antibody containing a heavy chain containing a heavy chain variable region consisting of an amino acid sequence of SEQ ID NO: 6 and a light chain containing a light chain variable region consisting of an amino acid sequence of SEQ ID NO: 8.

The present invention also provides an anti-CLDN4 antibody of the present invention or its antigen binding fragment to which a carbohydrate, a lipid, a metal (including a radioisotope), an organic compound (including a toxin, a near-infrared fluorescent dye, and a chelating agent) or the like (also referred to as a “modifier”) is bound (also referred to as a “complex of the present invention”). Herein, the term “modifier” refers to a nonpeptide substance bound to an antibody or its antigen binding fragment directly or via a linker and the like. A modifier for use in the complex of the present invention is not especially limited, and examples thereof include polyethylene glycol, sugar chains, phospholipids, radioactive isotopes (for example, zirconium-89 (⁸⁹Zr), yttrium-90 (⁹⁰Y), indium-111 (¹¹¹In), astatine-211 (²¹¹At), and actinium-225 (²²⁵Ac)), organic compounds, toxins, near-infrared fluorescent dyes (for example, IRDye®), and chelating agents. The modifier for use in the complex may be directly bound to the anti-CLDN4 antibody of the present invention or its antigen binding fragment or may be bound thereto via any linker. In one embodiment, the complex of the present invention is an antibody drug conjugate (ADC) of the anti-CLDN4 antibody or its antigen binding fragment. A chemical drug and a linker for use in the ADC can be chosen from drugs and linkers generally used by those skilled in the art.

The present invention also provides a cell expressing the anti-CLDN4 antibody of the present invention or its antigen binding fragment on a cell surface thereof (for example, a chimeric antigen receptor-T cell; CAR-T cell). Such a cell can be produced by those skilled in the art using a polynucleotide encoding the anti-CLDN4 antibody of the present invention or its antigen binding fragment. As the cell expressing the anti-CLDN4 antibody of the present invention or its antigen binding fragment, various immune cells (such as a T cell, a NK cell, and a NKT cell) can be used.

In one embodiment, the anti-CLDN4 antibody of the present invention or its antigen binding fragment, the complex of the present invention, and an antibody or antigen binding fragment portion in the cell expressing the anti-CLDN4 antibody of the present invention or its antigen binding fragment on a cell surface thereof may be post-translationally modified. In one embodiment, the post-translational modification is pyroglutamylation at the N-terminal of the heavy chain variable region and/or lysine deletion at the heavy chain C-terminal.

The anti-CLDN4 antibody of the present invention or its antigen binding fragment, the complex of the present invention, and the cell expressing the anti-CLDN4 antibody of the present invention or its antigen binding fragment on a cell surface thereof can be produced by those skilled in the art based on sequence information of the VHs and the VLs of the anti-CLDN4 antibody of the present invention or its antigen binding fragment and information of the modifier for use in the complex of the present invention disclosed herein by employing a method known in this field. The anti-CLDN4 antibody of the present invention or its antigen binding fragment can be produced in accordance with, for example, a method described in a section of <Method for Producing Bispecific Antibody of Invention>, although not especially limited thereto.

The present invention also provides a pharmaceutical composition containing the anti-CLDN4 antibody of the present invention or its antigen binding fragment, the complex of the present invention, and the cell expressing the anti-CLDN4 antibody of the present invention or its antigen binding fragment on a cell surface thereof (hereinafter collectively referred to as the “anti-CLDN4 antibody and the like of the present invention” in this section), and a pharmaceutically acceptable excipient. The pharmaceutical composition can be used for treating cancer. The present invention additionally provides a method for treating cancer including a step of administering a therapeutically effective amount of the anti-CLDN4 antibody and the like of the present invention to a subject, the anti-CLDN4 antibody and the like of the present invention for use in treating cancer, and use, in production of a pharmaceutical composition for treating cancer, of the anti-CLDN4 antibody and the like of the present invention. A pharmaceutical use of the anti-CLDN4 antibody and the like of the present invention can be carried out by those skilled in the art according to <Pharmaceutical Composition or the Like of Invention> described above. Examples of cancer to be treated by the pharmaceutical use of the anti-CLDN4 antibody and the like of the present invention include cancer described above in <Pharmaceutical Composition or the Like of Invention>.

### <Anti-CD137 Antibody of Invention>

The present invention also provides an anti-CD137 antibody or its antigen binding fragment containing a heavy chain variable region and a light chain variable region of an anti-CD 137 antibody described in any of the following (a) to (d):

(a) a heavy chain variable region containing a CDR1 consisting of an amino acid sequence at amino acid positions 31-35 of SEQ ID NO: 10, a CDR2 consisting of an amino acid sequence at amino acid positions 50-66 of SEQ ID NO: 10, and a CDR3 consisting of an amino acid sequence at amino acid positions 99-107 of SEQ ID NO: 10, and a light chain variable region containing a CDR1 consisting of an amino acid sequence at amino acid positions 24-34 of SEQ ID NO: 12, a CDR2 consisting of an amino acid sequence at amino acid positions 50-56 of SEQ ID NO: 12, and a CDR3 consisting of an amino acid sequence at amino acid positions 89-98 of SEQ ID NO: 12;

(b) a heavy chain variable region containing a CDR1 consisting of an amino acid sequence at amino acid positions 31-35 of SEQ ID NO: 14, a CDR2 consisting of an amino acid sequence at amino acid positions 50-65 of SEQ ID NO: 14, and a CDR3 consisting of an amino acid sequence at amino acid positions 98-107 of SEQ ID NO: 14, and a light chain variable region containing a CDR1 consisting of an amino acid sequence at amino acid positions 23-35 of SEQ ID NO: 16, a CDR2 consisting of an amino acid sequence at amino acid positions 51-57 of SEQ ID NO: 16, and a CDR3 consisting of an amino acid sequence at amino acid positions 90-100 of SEQ ID NO: 16;

(c) a heavy chain variable region containing a CDR1 consisting of an amino acid sequence at amino acid positions 31-35 of SEQ ID NO: 18, a CDR2 consisting of an amino acid sequence at amino acid positions 50-65 of SEQ ID NO: 18, and a CDR3 consisting of an amino acid sequence at amino acid positions 98-107 of SEQ ID NO: 18, and a light chain variable region containing a CDR1 consisting of an amino acid sequence at amino acid positions 23-35 of SEQ ID NO: 20, a CDR2 consisting of an amino acid sequence at amino acid positions 51-57 of SEQ ID NO: 20, and a CDR3 consisting of an amino acid sequence at amino acid positions 90-100 of SEQ ID NO: 20; or

(d) a heavy chain variable region containing a CDR1 consisting of an amino acid sequence at amino acid positions 31-35 of SEQ ID NO: 22, a CDR2 consisting of an amino acid sequence at amino acid positions 50-65 of SEQ ID NO: 22, and a CDR3 consisting of an amino acid sequence at amino acid positions 98-110 of SEQ ID NO: 22, and a light chain variable region containing a CDR1 consisting of an amino acid sequence at amino acid positions 23-35 of SEQ ID NO: 24, a CDR2 consisting of an amino acid sequence at amino acid positions 51-57 of SEQ ID NO: 24, and a CDR3 consisting of an amino acid sequence at amino acid positions 90-100 of SEQ ID NO: 24.

In one embodiment, the anti-CD137 antibody of the present invention or its antigen binding fragment contains a heavy chain variable region and a light chain variable region of an anti-CD137 antibody described in any of the following (a) to (i):
(a) a heavy chain variable region consisting of an amino acid sequence at amino acid positions 1-118 of SEQ ID NO: 10 and a light chain variable region consisting of an amino acid sequence at amino acid positions 1-109 of SEQ ID NO: 12;
(b) a heavy chain variable region consisting of an amino acid sequence at amino acid positions 1-118 of SEQ ID NO: 14 and a light chain variable region consisting of an amino acid sequence at amino acid positions 1-111 of SEQ ID NO: 16;
(c) a heavy chain variable region consisting of an amino acid sequence at amino acid positions 1-118 of SEQ ID NO: 18 and a light chain variable region consisting of an amino acid sequence at amino acid positions 1-111 of SEQ ID NO: 20;
(d) a heavy chain variable region consisting of an amino acid sequence at amino acid positions 1-121 of SEQ ID NO: 22 and a light chain variable region consisting of an amino acid sequence at amino acid positions 1-111 of SEQ ID NO: 24;
(e) a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26;
(f) a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28;
(g) a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30;
(h) a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32; or
(i) a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34.

In one embodiment, the antigen binding fragment of the anti-CD137 antibody of the present invention is a scFv. In one embodiment, the antigen binding fragment of the anti-CD137 antibody of the present invention is any of the following (a) to (e):
(a) an anti-CD137 scFv consisting of an amino acid sequence at amino acid positions 1-242 of SEQ ID NO: 26;
(b) an anti-CD137 scFv consisting of an amino acid sequence at amino acid positions 1-244 of SEQ ID NO: 28;
(c) an anti-CD137 scFv consisting of an amino acid sequence at amino acid positions 1-249 of SEQ ID NO: 30;
(d) an anti-CD137 scFv consisting of an amino acid sequence at amino acid positions 1-244 of SEQ ID NO: 32; or
(e) an anti-CD137 scFv consisting of an amino acid sequence at amino acid positions 1-247 of SEQ ID NO: 34.

The anti-CD137 antibody of the present invention or its antigen binding fragment can be produced by those skilled in the art with reference to <Anti-CLDN4/anti-CD137 Bispecific Antibody of Invention> and the like described herein. The anti-CD137 antibody of the present invention or its antigen binding fragment may be used in a bispecific antibody with an antibody to any TAA (anti-TAA antibody), for example, used in treatment of cancer. In other words, the present invention also provides a bispecific antibody containing the anti-CD137 antibody of the present invention or its antigen binding fragment and the anti-TAA antibody or its antigen binding fragment (also referred to as the “anti-TAA/anti-CD137 bispecific antibody of the present invention”). The TAA is not especially limited as long as it is expressed on a cell surface of a tumor, and for example, HER2, EGFR, EpCAM, CEA, BCMA, PSMA, CD19, CD20, CD22, CD33, CD37, CD38, CD123, CD276 (B7-H3), GPC2, GPC3, GPRC5D, WT-1, NY-ESO-1, CLDN4, CLDN6, CLDN18.2, and TSPAN8 can be used. In one embodiment, the anti-TAA/anti-CD137 bispecific antibody of the present invention contains a scFv of an anti-CD137 antibody, and in another embodiment, the anti-TAA/anti-CD137 bispecific antibody of the present invention contains a scFv of an anti-CD137 antibody described in any of the following (a) to (e):
(a) an anti-CD137 scFv consisting of an amino acid sequence at amino acid positions 1-242 of SEQ ID NO: 26;
(b) an anti-CD137 scFv consisting of an amino acid sequence at amino acid positions 1-244 of SEQ ID NO: 28;
(c) an anti-CD137 scFv consisting of an amino acid sequence at amino acid positions 1-249 of SEQ ID NO: 30;
(d) an anti-CD137 scFv consisting of an amino acid sequence at amino acid positions 1-244 of SEQ ID NO: 32; or
(e) an anti-CD137 scFv consisting of an amino acid sequence at amino acid positions 1-247 of SEQ ID NO: 34.

In one embodiment, the anti-TAA/anti-CD137 bispecific antibody of the present invention contains an anti-CLDN4 antibody or its antigen binding fragment, and in another embodiment, the anti-TAA/anti-CD137 bispecific antibody of the present invention contains an IgG type anti-CLDN4 antibody.

Specific examples to be referred to for further understanding of the present invention will now be provided, and it is noted that these examples are merely illustrative and do not limit the present invention.

### EXAMPLES

### Examples 1: Acquisition of Antibody Binding Selectively to Antigen Expressed in Peritoneal Metastatic Cancer Cell

[1-1. Acquisition of Peritoneal Metastatic Cancer Cell Derived from Patient]

Peritoneal metastatic cancer cells from a patient were obtained according to a method described in the literature by Fumiko Chiwaki and Hiroki Sasaki “Fukumaku Teni Gan (I, Sui, Ranso Gan Nado) Saibo Kabu No Juritsu (in Japanese) (Establishment of Peritoneal Metastasis Cancer (such as Gastric, Pancreatic, and Ovarian Cancer) Cell Lines)” (Practical Guide for Cancer Research using Patient-Derived Experimental Model, edited by Hiroki Sasaki, Yodosha Company, Ltd., 2019, p. 28-37).

Ascites fluid taken from the patient was centrifuged at room temperature and 430× g for 3 minutes, a supernatant was removed, and a hemolysis buffer was added to a precipitate to cause hemolysis. After centrifugation, the supernatant was removed, Dulbecco's PBS (−) (Nissui Pharmaceutical Co., Ltd., 05913; hereinafter referred to as the “PBS (−)”) was added in an amount of 50 mL to wash the cells. Thereafter, the resultant cells were collected by centrifugation at room temperature and 430×g for 3 minutes. The whole cells in the collected ascites fluid were suspended again in RPMI-1640 (containing L-glutamine) medium (FUJIFILM Wako Pure Chemical Corporation, 189-02025) containing 10% FBS (Thermo Fisher Scientific, 10270-106) and ×1 Antibiotic-Antimycotic (Thermo Fisher Scientific, 15240062) (wherein the medium after the addition of FBS and the like in Example 1 is hereinafter referred to as the “RPMI-1640 medium”). The suspension was diluted, and the cells were seeded at 5×10⁶ to 1×10⁷ cells/10 mL each in a 100 mm collagen coated dish (hereinafter referred to as the “dish”) (IWAKI, 4020-010) to be cultured at 37° C. in a 5% CO₂ incubator.

In the ascites fluid, there are adhesive cells and floating cells, and the adhesive cells include not only cancer cells but also non-cancer cells (such as fibroblasts and peritoneal mesothelial cells). The non-cancer cells have properties of detaching in a shorter time than the cancer cells, and thus these cells were removed by separating them from the whole cells in the ascites fluid. After removing the non-cancer cells, the cancer cells grew to approximately 80% confluency of the dish area, and then a process of passaging ½ of the whole cells to a new dish was repeated, and cells that had been passaged 5 times or more were considered adhesive cancer cells. For the floating cells, 5 ml of the culture supernatant and 5 ml of the RPMI-1640 medium were seeded into a new 100 mm dish from the culture dish to passage the cells, and cells that had been passaged 5 times or more were considered floating cancer cells. When peritoneal metastatic cancer cells derived from a single patient grew with both adhesive cancer cells and floating cancer cells, they were considered mixed cancer cells.

Herein, adhesive cancer cells, floating cancer cells, or mixed cancer cells isolated by the above method are generically referred to as the “peritoneal metastatic cancer cells.” The obtained six cells (NSC-9C, NSC-15CF, NSC-16C, NSC-20C, NSC-22C, and NSC-32C (hereinafter also referred to as the “six types of the peritoneal metastatic cancer cells”)) were used in the following examination.

### [1-2: Production of Anti-gastric Cancer Antigen-Antibody Producing Hybridoma]

The peritoneal metastatic cancer cells were immunized to VelocImmune mice produced using human monoclonal antibody development technology (“VelocImmune” ® antibody technology; Regeneron (U.S. Pat. No. 6,596,541)) to obtain antibodies against a cancer antigen expressed on an anti-peritoneal metastatic cancer cell.

Among the peritoneal metastatic cancer cells obtained in Example 1-1, three cells NSC-9C, NSC-15CF, and NSC-16C or three cells NSC-20C, NSC-22C, and NSC-32C were mixed, and the resultants were suspended in a TiterMax® Gold ADJUVANT (Merck, T2684) or PBS (−) to prepare a suspension of the peritoneal metastatic cancer cells. The VelocImmune mice were immunized by administering this suspension several times. Lymphocytes was collected from the lymph nodes of the immunized mice and were subjected to cellular fusion with mouse myeloma cells SP2/0 (ATCC, CRL-1581) to produce hybridomas by a conventional method. Single colonies of the hybridomas were isolated with an automatic picking device to obtain a monocloned hybridoma cell (hereinafter referred to as the “clone”). The isolated clone was cultured at 37° C. in an 8% CO₂ incubator, and a supernatant after about 14 days was collected in a 96 well plate to use the following experiment.

### [1-3: Selection of Antibody Binding Selectively to Peritoneal Metastatic Cancer Cell]

### (1) Check of Binding to Peritoneal Metastatic Cancer Cell and EpCAM Expressing Cell

A cell supernatant of the clone obtained in Example 1-2 contains an antibody (hereinafter referred to as the “antibody contained in the clone supernatant”).

First, binding of the antibody contained in the clone supernatant to the six types of the peritoneal metastatic cancer cells obtained in Example 1-1 was measured by flow cytometry to select a clone producing an antibody binding strongly to the peritoneal metastatic cancer cells. BV421 Goat Anti-Mouse Ig (Becton, Dickinson and Company, 563846) was used for flow cytometry.

Besides, the binding of the antibody contained in the clone supernatant to human EpCAM-Myc-DDK expressing CHO-K1 cells was measured for excluding clones providing an antibody binding to EpCAM that is a cancer antigen. The human EpCAM-Myc-DDK expressing CHO-K1 cells were produced by transfection of CHO-K1 cells (ATCC, CCL-61) with an EPCAM (Myc-DDK-tagged)-Human epithelial cell adhesion molecule (EPCAM) (ORIGENE, RC201989). The binding of the antibody contained in the clone supernatant to the cells was measured by flow cytometry. BV421 Goat Anti-Mouse Ig was used for flow cytometry. Clones binding to an EpCAM expressing cells were excluded for selecting clones providing a supernatant that did not exhibit binding activity to the cell. As a positive control, CD326 (EpCAM) Monoclonal Antibody (1B7) (eBioscience, 14-9326) was used.

Furthermore, clones binding to cultured human peritoneal mesothelial cells were excluded for selecting clones binding selectively to peritoneal metastatic cancer cells. As the cultured human peritoneal mesothelial cell, Human Mesothelial Cell (Zenbio, MES-F, Lot. MESMO50311A) (hereinafter also referred to as the “cultured human peritoneal mesothelial cell”) was used, and binding between the cultured human peritoneal mesothelial cells cultured in Mesothelial Cell Growth Medium (Zenbio, MSO-1) and a clone thereof was measured by flow cytometry. BV421 Goat Anti-Mouse Ig was used for flow cytometry. Only clones that did not bind to the cultured human peritoneal mesothelial cells were selected.

Through the above experiment, a 3D11 clone was obtained as a clone providing an antibody binding to 5 or more of the six peritoneal metastatic cancer cells but not to human EpCAM and cultured peritoneal mesothelial cells.

(2) Purification of Antibody from Hybridoma Supernatant

The 3D11 clone was cultured in a CD hybridoma medium (Thermo Fisher Scientific, 11279023). From culture supernatants thus obtained, a 3D11 antibody (hereinafter referred to as “3D11”) were purified with MabSelect SuRe (GE Healthcare, 17-5438-02). The antibody was purified by a conventional method.

### [1-4: Identification of Antigen Molecule Recognized by 3D11]

### (1) Identification of Candidate Antigen Molecule by LC-MS/MS Measurement

A candidate antigen molecule for 3D11 was identified. As a control antibody in this experiment, 1D10 and 10B5 were used. These antibodies showed a different binding pattern to peritoneal metastatic cancer cells than 3D11 in the process of obtaining 3D11.

A cell homogenate of NSC-15CF was prepared. To the cell homogenate, 3D11 and one of the two control antibodies (1D10, 10B5) were added, and Dynabeads Protein G (Life Technologies, 10003D) was further added thereto for stirring and washing. A protein bound to Dynabeads Protein G was digested using Trypsin/LysC (Promega Corporation, V5072) to obtain a peptide mixture. A solution containing the peptide mixture was subjected to LC-MS/MS measurement with UltiMate 3000 RSLCnano (Thermo Fisher Scientific) and Orbitrap Fusion (Thermo Fisher Scientific). The thus obtained LC-MS/MS data were subjected to comparative quantitative analysis and peptide/protein identification with Progenesis QI for Proteomics (Waters) and Mascot (Matrix Science) software to identify binding proteins. The binding proteins of 3D11 and the control antibodies were compared, and CLDN4 was identified as a candidate antigen molecule for 3D11.

### (2) Antigen Determination of 3D11

A binding experiment to human CLDN4-Myc-DDK expressing CHO-K1 cells was performed for determining whether the CLDN4 thus identified in (1) is an antigen of 3D11. Human CLDN4-Myc-DDK expressing CHO-K1 cells were produced by transfection of CHO-K1 cells with CLDN4 (Myc-DDK-tagged)-Human claudin 4 (CLDN4) (ORIGENE, RC200490). As a result, it was confirmed that 3D11 bound to the human CLDN4-Myc-DDK expressing CHO-K1 cells and thus recognized CLDN4 as an antigen.

### Examples 2: Sequence Determination of 3D11 and Production of Human Antibody

A gene encoding a heavy chain and a light chain of 3D11 was cloned by a conventional method to determine a nucleotide sequence and an amino acid sequence of 3D11. An amino acid sequence of a heavy chain variable region of 3D11 is shown in SEQ ID NO: 2, and an amino acid sequence of a light chain variable region thereof is shown in SEQ ID NO: 4. An antibody produced by VelocImmune mice are an antibody in which endogenous immunoglobulin heavy chain and light chain variable regions have been substituted with corresponding human variable regions. In other words, an antibody obtained by employing a VelocImmune technique irs an antibody having a variable region of a human antibody and a constant region of a mouse antibody (also referred to as a “chimeric antibody”).

### Reference Example: Anti-CLDN4/anti-CD3 Bispecific Antibody for Use in Treatment of Cancer Peritoneal Metastasis

Various types of drugs such as an antibody, an antibody-drug conjugate (ADC) antibody, and chimeric antigen receptor T-cell therapy (CAR-T) are known as a therapeutic agent for a TAA expressing selectively on a cancer cell, Among them, a bispecific T cell recruiting antibody (T cell recruiting antibody) is known as an innovative method by which cancer cell-selective cytotoxic activity can be obtained at a low antibody concentration. Therefore, a bispecific T cell recruiting antibody using 3D11 was examined.

### (1) Production of Humanized Anti-CLDN4 Antibody

A human anti-CLDN4/anti-CD3 scFv bispecific antibody was produced by the following method.

The human anti-CLDN4 antibody portion was designed by linking an IgG1 constant region of a human heavy chain to the heavy chain variable region of 3D11, and a constant region of the human light chain k to the light chain variable region by a conventional method. Besides, LALA mutation (L234 and L235A) in which each of amino acids at amino acid positions 238 and 239 (EU index: 234 and 235) of the heavy chain is substituted from leucine (L) to alanine (A), Knobs-into-holes mutation in which amino acids at amino acid positions 370, 372 and 411 (EU index: 366, 368 and 407) are respectively substituted from threonine (T) to serine(S), L to A, and tyrosine (Y) to valine (V), and mutation in which an amino acid at an amino acid position 301 (EU index: 297) is substituted from asparagine (N) to A were introduced. The thus produced humanized anti-CLDN4 antibody is referred to as 3D11.1. A polynucleotide encoding an amino acid sequence of 3D11.1 was synthesized by a conventional method and inserted into a pcDNA3.4 TOPO vector (Thermo Fisher Scientific).

(2) Production of Anti-CD3 scFv

An anti-CD3 scFv portion was produced based on the sequences of a heavy chain variable region and a light chain variable region of a mouse anti-CD3 antibody described in Japanese Patent No. 5686953. Humanization of the anti-CD3 scFv was performed according to a method described in the literature (Front Biosci., 2008, Vol. 13, p. 1619-1633). In this process, a back mutation was introduced. The three-dimensional structure information (PDB code: 5FCS) was analyzed by an integrated computational chemistry system MOE provided by MOLSIS Inc. to determine positions for introducing the back mutation in the framework region. An amino acid sequence of the humanized anti-CD3 scFv is shown in SEQ ID NO: 41. A polynucleotide encoding the amino acid sequence described in SEQ ID NO: 41 was synthesized by a conventional method and inserted into a pcDNA3.1 (+) vector (Thermo Fisher Scientific, V79020).

(3) Production of Anti-CLDN4 (3D11)/anti-CD3 scFv Bispecific Antibody

A vector encoding 3D11.1 and anti-CD3 scFv was used to produce a bispecific antibody composed of a Fab region of the anti-CLDN4 antibody and a scFv region and Fc region of the anti-CD3 antibody according to a method described in Example 7 of PCT/JP2021/41839. The thus produced bispecific antibody is referred to as the anti-CLDN4 (3D11)/anti-CD3 scFv bispecific antibody.

(4) Evaluation of Medical Effect of Anti-CLDN4 (3D11)/anti-CD3 scFv Bispecific Antibody

The in vitro redirected T cell cytotoxicity (RTCC) activity of the anti-CLDN4 (3D11)/anti-CD3 scFv bispecific antibody was measured according to a method described in Example 9 of PCT/JP2021/41839. It was confirmed that the anti-CLDN4 (3D11)/anti-CD3 scFv bispecific antibody has RTCC activity. Besides, the in vivo antitumor activity of the anti-CLDN4 (3D11)/anti-CD3 scFv bispecific antibody was evaluated according to a method described in Example 11 of PCT/JP2021/41839. The anti-CLDN4 (3D11)/anti-CD3 scFv bispecific antibody exhibited an antitumor effect in an in vivo mouse model with gastric cancer peritoneal metastasis. From these results, it was suggested that the anti-CLDN4 (3D11)/anti-CD3 scFv bispecific antibody is effective for the treatment of cancer.

Furthermore, in order to examine safety of the anti-CLDN4 (3D11)/anti-CD3 scFv bispecific antibodies, the anti-CLDN4 (3D11)/anti-CD3 scFv bispecific antibody was administered in a single dose intravenously to cynomolgus monkeys. As a result, death and dying examples were observed in the monkeys administered with the anti-CLDN4 (3D11)/anti-CD3 scFv bispecific antibody. Blood levels of IL-6 were remarkably increased in the dead monkeys after 1 hour and 6 hours of the administration, and in dying monkeys after 1 hour, 6 hours, and 24 hours of the administration. From these results, while the anti-CLDN4 (3D11)/anti-CD3 scFv bispecific antibody exerts medical effects in vitro and in vivo, a concern for a serious adverse reaction, presumably due to increased cytokines, has arisen. Therefore, it was concluded that the anti-CLDN4 (3D11)/anti-CD3 scFv bispecific antibody was not qualified as an antibody for treatment of human cancer.

### Examples 3: Production of Anti-CLDN4 (3D11)/anti-CD137 scFv Bispecific Antibody

A bispecific antibody with an anti-CD137 scFv (hereinafter referred to as the “anti-CLDN4 (3D11)/anti-CD137 scFv bispecific antibody”) was produced using 3D11 for examination.

### [3-1. Acquisition of Anti-CD137 Antibody]

### (1) Production of CD137-Fc Fusion Protein

Polynucleotides encoding an extracellular region of human CD137 consisting of an amino acid sequence at amino acid positions 1-186 of SEQ ID NO: 42 or an extracellular region of monkey CD137 consisting of an amino acid sequence at amino acid positions 1-186 of SEQ ID NO: 43 were respectively inserted into pFUSE-hIgG1-Fc1 vectors (InvivoGen, pfuse-hg1fc1) to produce expression vectors encoding fusion proteins of the extracellular regions of CD137 and human Fc (hereinafter respectively referred to as the “human CD137-human Fc fusion protein” and the “monkey CD137-human Fc fusion protein”). Each of the thus produced vectors was introduced into Expi293F cells (Thermo Fisher Scientific, A14527) or ExpiCHO-S cells (Thermo Fisher Scientific, A29133) with an Expi293 Expression System Kit (Thermo Fisher Scientific, A14635) or an ExpiCHO Expression System Kit (Thermo Fisher Scientific, A29133) for culturing. Proteins secreted into culture supernatants were purified with MabSelect SuRe or HiTrap MabSelect SuRe (GE Healthcare, 11-0034-94) to obtain human CD137-human Fc fusion protein and monkey CD137-human Fc fusion protein.

### (2) Production of Monkey CD137-His Tag-3×FLAG Tag Fusion Protein

Polynucleotides encoding an extracellular region of monkey CD137 consisting of an amino acid sequence at amino acid positions 1-183 of SEQ ID NO: 43, a His tag of SEQ ID NO: 44, and a 3×FLAG tag of SEQ ID NO: 45 were inserted in tandem into a pcDNA3.4 TOPO vector to produce a vector. The thus produced vector was introduced into ExpiCHO-S cells with an ExpiFectamine CHO Transfection Kit (Thermo Fisher Scientific, A29129) for culture. A protein secreted into a culture supernatant was purified with HisTrap excel (GE Healthcare Bioscience, 17-3712-06). The thus obtained fusion protein is referred to as the “monkey CD137-His tag-3×FLAG tag fusion protein.”

### (3) Production and Screening of Anti-CD137 Antibody Producing Hybridoma Clone

VelocImmune mice or AlivaMab mice (Ablexis, U.S. Pat. No. 9,346,873) were immunized by administering the human CD137-human Fc fusion protein or human CD137-His tag protein (R&D Systems, 9220-4B) and immunoadjuvants several times. Lymphocytes were collected from the lymph nodes of the immunized mice by a conventional method and were subjected to cellular fusion with mouse myeloma cells SP2/0 to produce hybridomas. Single colonies of the hybridomas were isolated with an automatic picking device to obtain monocloned hybridoma cells (hereinafter referred to as the “clone”). Each clone was cultured for several days to obtain a culture supernatant. Binding to CD137 of antibodies contained in the culture supernatant was checked by the following method.

The binding to CD137 was evaluated by ELISA. In the evaluation, a plate coated with any one of the human CD137-His tag protein, the human CD137-human Fc fusion protein, the monkey CD137-human Fc fusion protein, and human Fc protein was used, and Goat Anti-Mouse IgG, Human ads-HRP (SouthernBiotech, 1030-05) was used as a secondary antibody. By this method, a plurality of clones binding to both human CD137 and monkey CD137 were obtained.

### (4) Acquisition of Anti-CD137 Antibody and Check of Agonistic Activity

The clones obtained in (3) were cultured in Hybridoma-SFM (Thermo Fisher Scientific, 12045-076), and from a culture supernatant thus obtained, anti-CD137 antibodies were purified with MabSelect SuRe. The purified anti-CD137 antibodies were checked for binding to CD137 and CD137 agonistic activity.

The binding to CD137 of the anti-CD137 antibodies was checked by ELISA described in (3). Measurement of the CD137 agonistic activity of the anti-CD137 antibodies was performed with 4-1BB Bioassay Kit (Promega Corporation, JA2351) by the following method. FcγRIIb CHO-K1 Cells (Promega Corporation, JA2251) were seeded in a 384 well plate (Greiner Bio-One, 781080) to be cultured at 37° C. and 5% CO₂ overnight. A culture supernatant was removed, the purified antibodies of each clone described above and 4-1BB Effector Cells (Promega Corporation, JA2351) were added thereto, and the resultant was allowed to stand still at 37° C. and 5% CO₂ for 6 hours. To the resultant, Bio-Glo Reagent (Promega Corporation, JA2351) was added to measure chemiluminescence by luciferase expression induction in response to CD137 agonistic activity. The same experiment as above was performed under FcγRIIb CHO-K1 Cells-free conditions for evaluating the CD137 agonistic activity in the absence of FcγRIIb CHO-K1 Cells.

Among the anti-CD137 antibodies thus evaluated, 3-34 derived from VelocImmune mice, and A2-32, A2-48, and A2-73 derived from AlivaMab mice had strong binding activity to human and monkey CD137 and exhibited CD137 agonistic activity dependent on the presence of FcγRIIb CHO-K1 Cells.

### (5) Identification of Gene Sequence of Anti-CD137 Antibody

From each of the clones providing the anti-CD137 antibodies (3-34, A2-32, A2-48, and A2-73), a cell lysate was prepared by a conventional method, a cDNA was synthesized, and an antibody nucleotide sequence was identified. SEQ ID NO of the nucleotide sequence thus identified is shown in Table 1.

TABLE 1

| | Nucleotide sequence of heavy | Nucleotide sequence of light |
|---|---|---|
| | chain variable region | chain variable region |
| 3-34 | SEQ ID NO: 9 | SEQ ID NO: 11 |
| A2-32 | SEQ ID NO: 13 | SEQ ID NO: 15 |
| A2-48 | SEQ ID NO: 17 | SEQ ID NO: 19 |
| A2-73 | SEQ ID NO: 21 | SEQ ID NO: 23 |

[3-2. Production of Anti-CLDN4 (3D11)/anti-CD137 (3-34) scFv Bispecific Antibody]

The anti-CLDN4 (3D11)/anti-CD137 (3-34) scFv bispecific antibody (hereinafter also referred to as “3D11_3-34-j6_S”) was designed based on the sequences of the heavy chain variable region and the light chain variable region of 3D11 and 3-34. In 3D11_3-34-j6_S thus produced, a GS linker is linked to the heavy chain carboxy terminal (also referred to as the “C-terminal”) of the anti-CLDN4 (3D11) antibody of the human IgG1κ type, and the amino terminal of an anti-CD137 (3-34) scFv (SEQ ID NO: 26) containing the heavy chain variable region and the light chain variable region of the anti-CD137 antibody is bounded to the C-terminal of the GS linker. Besides, amino acid mutations L234A, L235A, and P331S were introduced into the human IgG1 constant region of 3D11. Polynucleotides encoding the designed antibody were produced and inserted into pcDNA3.4 TOPO vectors by a conventional method. The expression vectors were genetically introduced into ExpiCHO-S cells, and 3D11_3-34-j6_S was purified from the culture supernatant. It was confirmed that 3D11_3-34-j6_S thus produced had anti-CD137 binding activity and CD137 agonistic activity in vitro, thus suggesting that it can be expected to be effective as a cancer drug.

### Examples 4: Production of Anti-CLDN4 (hKM3900)/anti-CD137 scFv Bispecific Antibody

[4-1. Production of Humanized Anti-CLDN4 (hKM3900) Antibody]

It has been reported that KM3900 that is an anti-CLDN4 antibody binds selectively to CLDN4 as compared with other claudin family molecules such as CLDN6 (PTL 1). Therefore, a humanized antibody of KM3900 was produced based on this report. Specifically, based on a humanized amino acid sequence of a variable region of KM3900, a humanized antibody was designed from sequences of a human Igγ1 constant region and a human Igκ constant region. Into the human Igγ1 constant region, an amino acid mutation L234A, L235A and P331G, or amino acid mutations L234A, L235A, and P331S were introduced. A humanized anti-CLDN4 (hKM3900) antibody was produced by a conventional method to be used in the following examination. An antibody having the amino acid mutations L234A, L235A, and P331G is referred to as “hKM3900,” and an antibody having amino acid mutations L234A, L235A and P331S is referred to as “hKM3900_S.” An amino acid sequence of a heavy chain variable region of hKM3900 is described in SEQ ID NO: 6, and an amino acid sequence of a light chain variable region thereof is described in SEQ ID NO: 8.

[4-2. Production of Anti-CLDN4/anti-CD137 scFv Bispecific Antibody]

Anti-CD137 scFvs were designed from the anti-CD137 antibodies obtained in Example 3 (3-34, A2-32, A2-48, and A2-73) to produce bispecific antibodies of hKM3900 and the anti-CD137 scFvs (Table 2). The thus produced antibody is generically referred to as the “anti-CLDN4 (hKM3900)/anti-CD137 scFv.”

(1) Production of Vector Encoding Anti-CLDN4 (hKM3900)/anti-CD137 scFv Bispecific Antibody

The anti-CLDN4 (hKM3900)/anti-CD137 scFv bispecific antibody was designed based on the sequence of hKM3900 and the sequences of the heavy chain variable region and the light chain variable region of the anti-CD137 antibody. In the anti-CLDN4 (hKM3900)/anti-CD137 scFv bispecific antibody, a GS linker is linked to the heavy chain or light chain C-terminal of the anti-CLDN4 (hKM3900) of the IgG1 type, and the amino terminal of an anti-CD137 scFv containing the heavy chain variable region and the light chain variable region of the anti-CD137 antibody is bound to the C-terminal of the GS linker. Besides, amino acid mutations L234A, L235A, and P331G or P331S were introduced into the human IgG1 constant region of hKM3900. Information on the designed antibodies is described in Table 2. Each of the polynucleotides encoding the designed antibodies was produced and inserted into a pcDNA3.4 TOPO vector by a conventional method. The thus produced vector is generically referred to as the “anti-CLDN4 (hKM3900)/anti-CD137 scFv bispecific antibody expression vector.”

TABLE 2
Anti-CLDN4 (hKM3900)/anti-CD137 scFv Bispecific Antibody

| | Anti-CLDN4 | Anti-CD137 | Anti-CD137 | | Fc |
|---|---|---|---|---|---|
| Antibody name | antibody | antibody | scFv | scFv-linked position | mutation |
| hKM3900_3-34-j6 | hKM3900 | 3-34 | SEQ ID NO: 26 | Heavy chain C-terminal | P331G |
| | | | 3-34-j6 | (*) | |
| hKM3900_3-34-j6_S | | 3-34 | SEQ ID NO: 26 | Heavy chain C-terminal | P331S |
| | | | 3-34-j6 | | |
| hKM3900_L-3-34-j6 | | 3-34 | SEQ ID NO: 26 | Light chain C-terminal | P331G |
| | | | 3-34-j6 | | |
| hKM3900_tA2-32 | | A2-32 | SEQ ID NO: 28 | Heavy chain C-terminal | P331G |
| | | | tA2-32 | | |
| hKM3900_tA2-32LH | | A2-32 | SEQ ID NO: 30 | Heavy chain C-terminal | P331G |
| | | | tA2-32LH | | |
| hKM3900_tA2-48 | | A2-48 | SEQ ID NO: 32 | Heavy chain C-terminal | P331G |
| | | | tA2-48 | | |
| hKM3900_tA2-73 | | A2-73 | SEQ ID NO: 34 | Heavy chain C-terminal | P331G |
| | | | tA2-73 | | |
| hKM3900_tA2-32_S | | A2-32 | SEQ ID NO: 28 | Heavy chain C-terminal | P331S |
| | | | tA2-32 | | |
| hKM3900_tA2-32LH_S | | A2-32 | SEQ ID NO: 30 | Heavy chain C-terminal | P331S |
| | | | tA2-32LH | | |
| hKM3900_tA2-48_S | | A2-48 | SEQ ID NO: 32 | Heavy chain C-terminal | P331S |
| | | | tA2-48 | | |
| hKM3900_tA2-73_S | | A2-73 | SEQ ID NO: 34 | Heavy chain C-terminal | P331S |
| | | | tA2-73 | | |

(*) C-terminal in the table means the carboxy terminal.

(2) Production of Anti-CLDN4 (hKM3900)/Anti-CD137 scFv Bispecific Antibody

The anti-CLDN4 (hKM3900)/anti-CD137 scFv bispecific antibody expression vectors were used to produce eleven anti-CLDN4 (hKM3900)/anti-CD137 scFv bispecific antibodies described in Table 2. Specifically, the anti-CLDN4 (hKM3900)/anti-CD137 scFv bispecific antibody expression vectors were introduced into ExpiCHO-S cells with ExpiFectamine CHO Transfection Kit to cause the anti-CLDN4/anti-CD137 scFv bispecific antibodies to be secreted into a culture supernatant. From the culture supernatant thus obtained, the anti-CLDN4 (hKM3900)/anti-CD137 scFv bispecific antibodies were purified by affinity purification with MabSelect SuRe. Furthermore, some of the anti-CLDN4 (hKM3900)/anti-CD137 scFv bispecific antibodies were additionally purified by size exclusion chromatography purification with HiLoad 26/600 superdex 200 pg (GE Healthcare, 28-9893-36) or Superdex 200 Increase 10/300 GL (GE Healthcare, 28-9909-44).

Hereinafter, the anti-CLDN4 (3D11)/anti-CD137 (3-34) scFv bispecific antibody and the anti-CLDN4 (hKM3900)/anti-CD137 scFv bispecific antibody produced in Examples 3 and 4 are generically referred to as the “anti-CLDN4/anti-CD137 scFv bispecific antibody” in some cases.

### Examples 5: Evaluation of Binding Activity and Agonistic Activity of Anti-CLDN4 (hKM3900)/anti-CD137 scFv Bispecific Antibody

The anti-CLDN4 (hKM3900)/anti-CD137 scFv bispecific antibody was evaluated for the binding activity to CLDN4 and CD137, and the CD137 agonistic activity.

[5-1. Evaluation of Binding Activity to CLDN4 of Anti-CLDN4 (hKM3900)/anti-CD137 scFv Bispecific Antibody by Flow Cytometry]

The binding activity to CLDN4 of the anti-CLDN4 (hKM3900)/anti-CD137 scFv bispecific antibody was evaluated by flow cytometry. A CLDN4 gene prepared from CLDN4 (Myc-DDK-tagged)-Human claudin 4 was inserted into pCMV6-AC-GFP Mammalian Expression Vector (ORIGENE, PS100010) to produce an expression vector for human CLDN4. The expression vectors for human CLDN4 were introduced into CHO-K1 cells to obtain human CLDN4 expressing CHO-K1.

As a test antibody, hKM3900 and the two anti-CLDN4 (hKM3900)/anti-CD137 scFv bispecific antibodies (hKM3900_tA2-32 LH and hKM3900_3-34-j6) were used. Each of the test antibodies was serially diluted with Stain Buffer (FBS) (BD Bioscience, 554656) from a maximum concentration of 100 μg/mL to 5.08 ng/mL with a common ratio of 3, and the human CLDN4 expressing CHO-K1 was stained with each test antibody dilute solution. As a secondary antibody, Allophycocyanin (APC) AffiniPure F(ab′)2 Fragment Goat Anti-Human IgG, Fcγ fragment specific (Jackson ImmunoResearch, 109-136-170) was used. Fluorescence intensity was measured with FACS Canto II (BD Bioscience). Measurement results were analyzed with GraphPad Prism to calculate an apparent dissociation constant (KD) between human CLDN4 and the anti-CLDN4 (hKM3900)/anti-CD137 scFv bispecific antibody. Values of the apparent KD thus obtained are shown in Table 3.

The two anti-CLDN4 (hKM3900)/anti-CD137 scFv bispecific antibodies exhibited the same binding activity to human CLDN4 as the humanized anti-CLDN4 (hKM3900) antibody. Other anti-CLDN4 (hKM3900)/anti-CD137 scFv bispecific antibodies are also expected to exhibit CLDN4 binding activity equivalent thereto.

TABLE 3

| | hKM3900 | hKM3900_tA2-32LH | hKM3900_3-34-j6 |
|---|---|---|---|
| KD (nM) | 10.8 | 10.8 | 11.2 |

[5-2. Evaluation of Binding Activity to CD137 of Anti-CLDN4 (hKM3900)/anti-CD137 scFv Bispecific Antibody by ELISA]

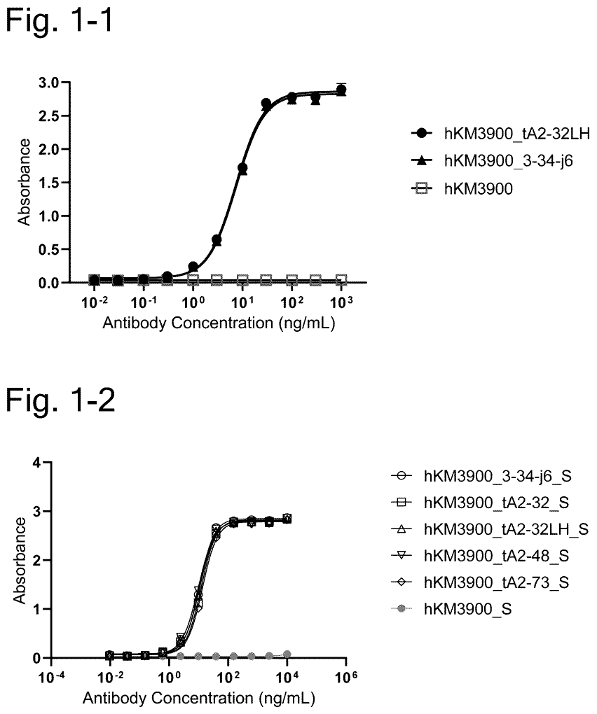

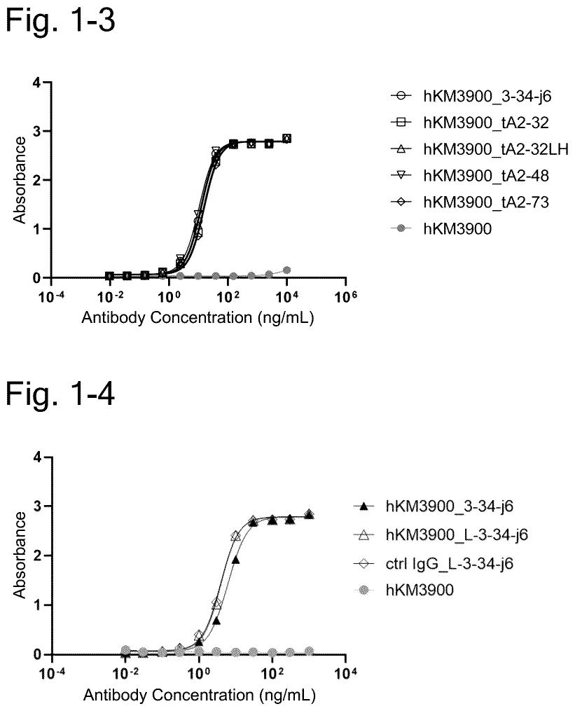

The binding activity to CD137 of the anti-CLDN4 (hKM3900)/anti-CD137 scFv bispecific antibody was detected by ELISA. The ELISA was performed by a conventional method. A 20 ng/20 μL/well human CD137-His tag protein (R&D SYSTEMS, 9220-4B) was immobilized on a 384 well plate (Thermo Fisher Scientific, 464718). As a test antibody, 11 anti-CLDN4 (hKM3900)/anti-CD137 scFv bispecific antibodies and a ctrl IgG_L-3-34-j6 antibody shown in Table 2 were used. The ctrl IgG_L-3-34-j6 antibody is an antibody in which an anti-CD137 scFv (3-34-j6) antibody is linked to a light chain carboxy terminal of an isotype control antibody. The test antibodies were serially diluted with TBS-T (Nippon Gene Co., Ltd., 310-07375) containing 5% Blocking One (Nacalai Tesque, Inc., 03953-95), and added in an amount of 20 μL/well. The antibody has a final concentration as illustrated in FIGS. 1-1 to 1-4. As a secondary antibody, Goat Anti-Human IgG Fc, Multi-Species SP ads-HRP (SouthernBiotech, 2014-05) was used. As a control antibody, hKM3900 was used. Infinite M200 PRO (TECAN) was used to measure absorbances at 450 nm and 570 nm. Results are illustrated in FIGS. 1-1 to 1-4. The anti-CLDN4 (hKM3900)/anti-CD137 scFv bispecific antibodies all bound to the human CD137-His tag protein in a concentration dependent manner. From this result, it was revealed that a linked position of the anti-CD137 scFv did not affect the binding activity to human CD137.

[5-3. Evaluation of Binding Activity to CD137 of Anti-CLDN4 (hKM3900)/anti-CD137 scFv Bispecific Antibody by Surface Plasmon Resonance]

The binding activity to CD137 of the anti-CLDN4 (hKM3900)/anti-CD137 scFv bispecific antibody was detected by surface plasmon resonance (SPR). For SPR analysis, Biacore T200 (GE Healthcare Japan) was used. As a ligand, one obtained by fixing two anti-CLDN4 (hKM3900)/anti-CD137 scFv bispecific antibodies (hKM3900-3-34-j6, hKM3900_tA2-32 LH) to Series S Sensor Chip Protein A (GE Healthcare, 29-1275-56) was used. As an analyte, one obtained by serially diluting human CD137-His tag or the monkey CD137-His tag-3×FLAG tag fusion protein produced in 3-1 (2) with HBS-EP+ from a maximum concentration of 200 nM to 0.0977 nM with a common ratio of 2 was used. The analytes were added to the channel having the ligand bound thereto at 50 μL/min for 2 minutes, then HBS-EP+ was added thereto at 50 μL/min for 10 minutes, and thus, binding and dissociation of the anti-CLDN4 (hKM3900)/anti-CD137 scFv bispecific antibody to and from CD137 were measured. Results obtained in this experiment were used to calculate, with data analysis software (BIA Evaluation), a binding rate constant (ka), a dissociation rate constant (kd), and a dissociation constant (KD) between human CD137 and the anti-CLDN4 (hKM3900)/anti-CD137 scFv bispecific antibody. Values of ka, kd, and KD are shown in Table 4.

As the result, it was revealed that the anti-CLDN4 (hKM3900)/anti-CD137 scFv bispecific antibody exhibited binding activity equivalent to both human CD137 and monkey CD137 antibodies.

TABLE 4

| | Ligand (only antibody | | | |
|---|---|---|---|---|
| Analyte | listed) | ka (1/Ms) | kd (1/s) | KD (M) |
| Human CD137-His | hKM3900_3-34-j6 | 2.32E+05 | 1.35E−03 | 5.81E−09 |
| Tag Protein | hKM3900_tA2-32LH | 3.74E+05 | 2.19E−03 | 5.84E−09 |
| Monkey CD137- | hKM3900_3-34-j6 | 1.79E+05 | 1.91E−03 | 1.07E−08 |
| His-3 × FLAG | hKM3900_tA2-32LH | 2.88E+05 | 1.92E−03 | 6.68E−09 |

[5-4. Evaluation of CD137 Agonistic Activity of Anti-CLDN4/anti-CD137 scFv Bispecific Antibody in Coexistence of CLDN4 Expressing Cancer Cell]

Specific CD137 agonistic activity via CLDN4 expressing cancer cells of the anti-CLDN4/anti-CD137 scFv bispecific antibody was evaluated with 4-1BB Bioassay Kit.

NCI-H322 cells (ECACC, 95111734) were suspended in RPMI-1640 medium (Thermo Fisher Scientific, 11875-093) containing 10% FBS (Cytiva, SH30070.03) to prepare a suspension. CLDN4 was expressed in NCI-H322. The suspension of NCI-H322 was added to a 384 well plate (Greiner Bio-One, 781080) at a density of 1×10⁴ cells/well, and the resultant was cultured at 37° C. and 5% CO₂ overnight. On the next day, the medium was removed. A test antibody diluted with 1% FBS containing RPMI-1640 medium was added in an amount of 12.5 μL/well each. The antibody has a final concentration as illustrated in FIG. 2-1 to FIG. 2-3. As the test antibody, 3D11_3-34-j6_S and the anti-CLDN4 (hKM3900)/anti-CD137 scFv bispecific antibody described in Table 2 were used. As a control antibody, hKM3900, a ctrl IgG_L-3-34-j6 antibody, and Urelumab (BMS, SEQ ID NO: 3 and SEQ ID NO: 6 described in International Publication No. WO2005/035584) were used.

A 4-1BB Effector Cells provided with the 4-1BB Bioassay Kits were suspended in 7 mL of 1% FBS-containing RPMI-1640 per vial, the resultant suspension was added in an amount of 12.5 μL to each well, and the resultant was allowed to stand still at 37° C. and 5% CO₂ for 6 hours. Bio-Glo Reagent was added to the plate in an amount of 25 μL/well, and chemiluminescence was measured with EnVision 2103 (Perkin Elmer). Results are illustrated in FIG. 2-1 to FIG. 2-3. The anti-CLDN4/anti-CD137 scFv bispecific antibody exhibited remarkable CD137 agonistic activity to the 4-1BB Effector Cells in the coexistence of the CLDN4 expressing cancer cells.

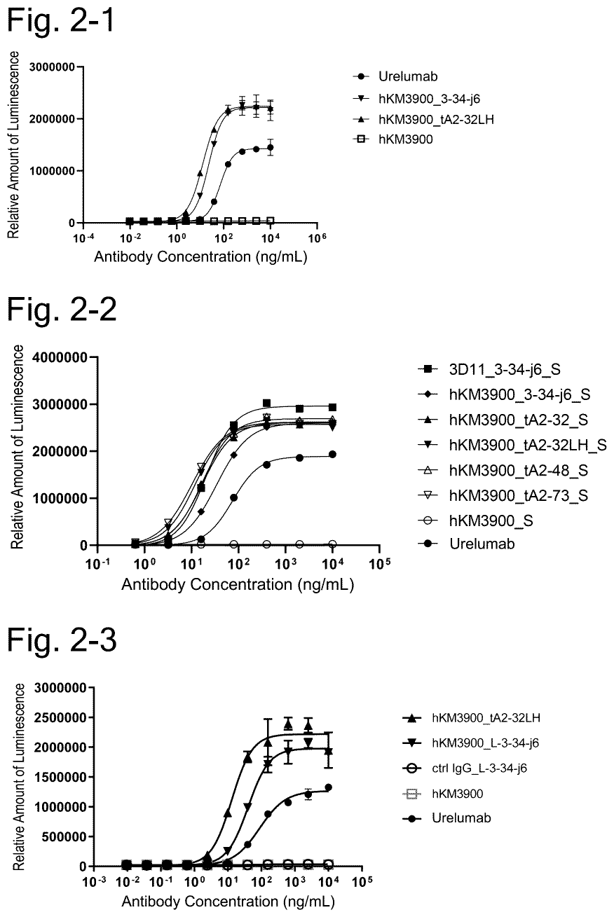

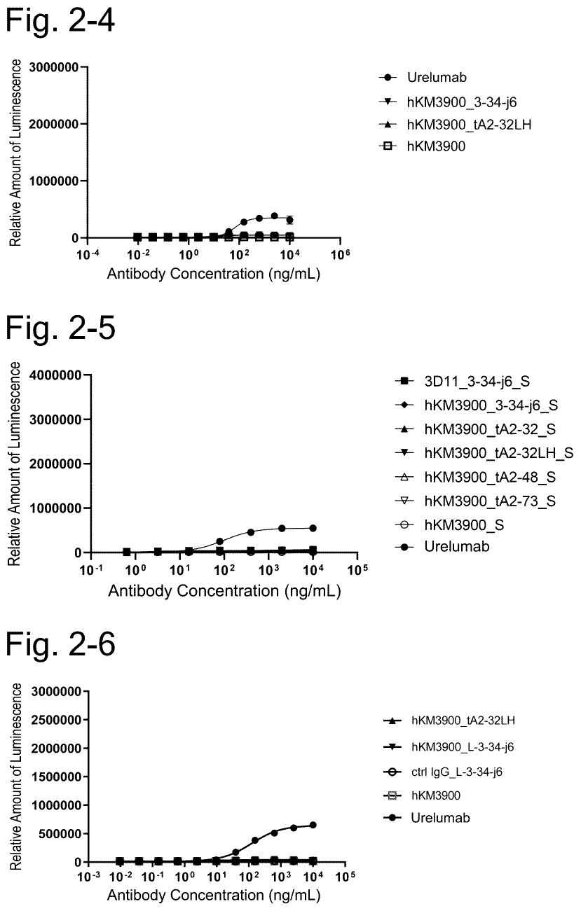

The same experiment as above was performed under NCI-H322-free conditions for evaluating the CD137 agonistic activity in the absence of the CLDN4 expressing cancer cells. As a result, it did not exhibit CD137 agonistic activity in the absence of the CLDN4 expressing cancer cells (FIG. 2-4 to FIG. 2-6). From this result, it was shown that the anti-CLDN4/anti-CD137 scFv bispecific antibody exerts CD137 agonistic activity only in the coexistence of the CLDN4 expressing cancer cells.

On the other hand, Urelumab exhibited CD137 agonistic activity in the coexistence of the CLDN4 expressing cancer cells, but the action was weak as compared with that of the anti-CLDN4/anti-CD137 bispecific antibody. Besides, Urelumab exhibited CD137 agonistic activity even in the absence of the CLDN4 expressing cancer cells.

From these results, the anti-CLDN4/anti-CD137 bispecific antibody can be expected to have a lower toxicity concern than Urelumab and exhibit specific and strong CD137 agonistic activity in a tumor tissue in which CLDN4 is highly expressed.

### Example 6: Evaluation of In Vitro Effect of Anti-CLDN4/Anti-CD137 scFv Bispecific Antibody

Evaluation of interferon-γ production promoting function and cancer cytotoxic activity of the anti-CLDN4/anti-CD137 scFv bispecific antibody were carried out.

### [6-1. Production and Cryopreservation of Expanded Pan T Cell]

### (1) Preparation of Expanded Pan T Cell

In Example 6, one obtained by adding FBS (Cytiva, SH30084.03) in a final concentration of 10% and penicillin streptomycin (Thermo Fisher Scientific, 15070-063) in a final concentration of 1% to RPMI-1640 (Sigma, R8758) is referred to as the “culture medium.” Each of MEM Non-essential Amino Acid (Merck, M7145), Sodium pyruvate (Merck, S8636), GlutaMAX I (Thermo Fisher Scientific, 35050-061), and HEPES (Thermo Fisher Scientific, 15630-080) may be added to the culture medium in a final concentration of 1%. An anti-CD3 antibody (BioLegend, 317325) diluted to a final concentration of 1 to 3 μg/mL was added to a dish, and the anti-CD3 antibody was immobilized. Pan T Cell Isolation Kit, human (Miltenyi Biotec, 130-096-535) was used to isolate Pan T cells (including both CD4T cells and CD8T cells; hereinafter referred to as the “Pan T cells”) from human peripheral blood mononuclear cells (LONZA, CC-2702) in accordance with a protocol recommended by the manufacturer. The isolated Pan T cells were centrifuged, a supernatant was removed, and the resultant was suspended in the culture medium. The whole Pan T cells suspended in the culture medium were seeded in the dish in which the anti-CD3 antibody had been immobilized. Besides, human IL-2 (PeproTech, 200-2) in a final concentration of 100 to 200 U/mL and an anti-CD28 antibody (BioLegend, 302923) in a final concentration of 1 to 4 μg/mL were added thereto, and the resultant was cultured at 37° C. in a 5% CO₂ incubator. After 3 days, the resultant cells were collected, the collected cell s were suspended in the culture medium, and seeded in a dish. Furthermore, human IL-2 in a final concentration of 100 to 200 U/mL was added thereto, and the resultant was cultured at 37° C. in a 5% CO₂ incubator. After 4 days, the whole cells were collected to be centrifuged, a supernatant was removed, the resultant was suspended again in a Cell Banker (Takara, CB011) or Bambanker (GC LYMPHOTEC Inc., CS-02-001), and the resultant was separated into tubes to be cryopreserved at −80° C. The cells thus cryopreserved are herein referred to as the “expanded Pan T cells.”

### [6-2. Production of 60As6-Luc/GFP Expressing Cell]

Preparation of a lentivirus solution containing Luc/GFP was carried out with L293T cells (Thermo Fisher Scientific, K4975-00) by a conventional method. For an introduction of pCDH-CMV-GL3-EFla-GFP-T2A-puro based vector (donated by Associate Professor Ryouu Takahashi at Hiroshima University, Graduate School of Biomedical and Health Sciences, Department of Cellular and Molecular Biology (PLOS One, 2015, Vol. 10, e0123407)) into the L293T cells, Opti-MEM Reduced Serum Medium, GlutaMAX Supplement (Thermo Fisher Scientific, 51985-034), MISSION® Lentiviral Packaging Mix (SIGMA, SHP001), and Lipofectamin LTX (Thermo Fisher Scientific, 15338100) were used. For the collection of a GFP expressing lentivirus grown in L293T cells, a 45 μm Millex®-HV filter (Merck Millipore, SLHV033RS) was used. The virus solution was dispensed into a cryovial (Nalgene, 5000-0020) by 1 mL each to be cryopreserved at −80° C.

A CLDN4 expressing cancer cell line, 60As6 cells (received from Dr. Kazuyoshi Yanagihara at Institute of Biomaterials) was infected with a 60As6-Luc/GFP expressing lentivirus by a conventional method to produce 60As6-Luc/GFP expressing cells. Polybrene (SantaCruz, sc-134220) was used for increasing infection efficiency. As a medium, RPMI-1640 containing 10% FBS and 2 μg/mL Puromaycin (Thermo Fisher Scientific, A-11138-02) was used. The thus established cells will be herein referred to as the “60As6-Luc/GFP cells.” It was confirmed through flow cytometry that CLDN4 was intrinsically highly expressed in the 60As6-Luc/GFP cells. The thus constructed cells were used in later experiments.

[6-3. Evaluation of Interferon-γ Production Promoting Function of Anti-CLDN4 (3D11)/anti-CD137 scFv Bispecific Antibody in Coculture System of Cancer Cell and T Cell]

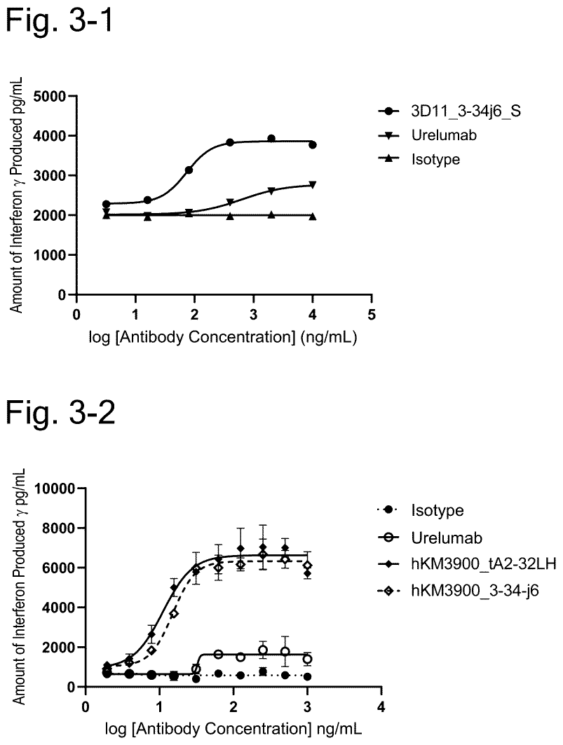

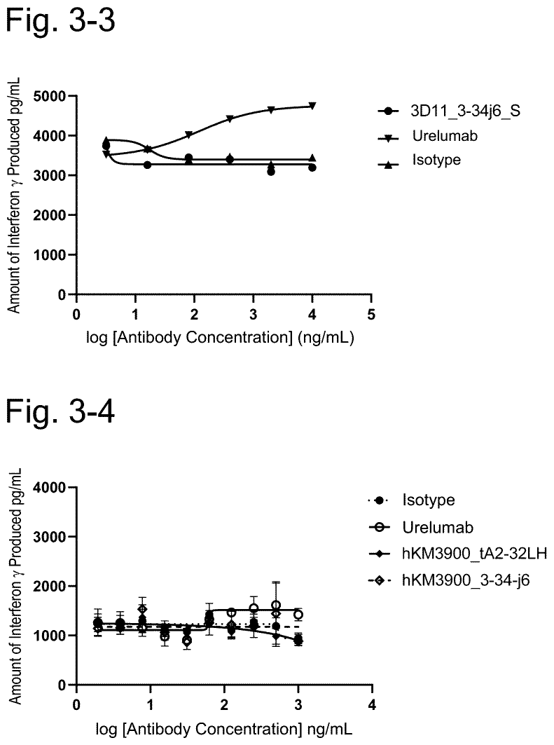

The interferon-γ production promoting function of the anti-CLDN4 (3D11)/anti-CD137 scFv bispecific antibody in a coculture system of the 60As6-Luc/GFP cells and the expanded Pan T cells was evaluated. A 0.1 μg/100 μL/well anti-CD3 antibody was added to a flat bottom 96 well plate (IWAKI, 3860-096) to immobilize the anti-CD3 antibody on the plate. The 60As6-Luc/GFP cells were seeded in the plate at 5×10⁴ cells/50 μL/well to be cultured at 37° C. in a 5% CO₂ incubator. On the next day, the expanded Pan T cells were seeded in the plate at 2× 10⁵ cells/30 μL/well. The test antibodies and the isotype control were serially diluted with the culture medium from a maximum concentration of 50000 ng/ml with a common ratio of 5 and added in an amount of 20 μL each. As a test antibody, Urelumab and the anti-CLDN4 (3D11)/anti-CD137 scFv bispecific antibody (3D11_3-34-j6_S) were used, and as an isotype control, an IgG (lysozyme)-scFv (CD137) (prepared in house) was used. After 6 days, a production amount of interferon γ in a supernatant was measured with AlphaLISA Interferon-γ Measurement Kit (Perkin Elmer, AL217C) in accordance with a protocol recommended by the manufacturer. FIG. 3-1 illustrates a reaction curve of the production amount of interferon γ and the antibody concentration. The anti-CLDN4 (3D11)/anti-CD137 scFv bispecific antibody exhibited interferon-γ production promoting activity in a coculture system of the CLDN4 expressing cancer cell line, the 60As6-Luc/GFP cells, and the expanded Pan T cells. The interferon-γ production promoting function of the anti-CLDN4 (3D11)/anti-CD137 scFv bispecific antibodies was remarkably stronger than that of Urelumab.

[6-4. Evaluation of Interferon-γ Production Promoting Function of Anti-CLDN4 (hKM3900)/anti-CD137 scFv Bispecific Antibody in Coculture System of Cancer Cell and T Cell]

The interferon-γ production promoting function of the anti-CLDN4 (hKM3900)/anti-CD137 scFv bispecific antibody in a coculture system of the 60As6-Luc/GFP cells and the expanded Pan T cells was evaluated by the same method as in 6-3. An anti-CD3 antibody was immobilized on a 96 well plate at 0.03 μg/100 μL/well. The 60As6-Luc/GFP cells were seeded at 2×10⁴ cells/50 μL/well. The expanded Pan T cells were seeded at 8×10⁴ cells/30 μL/well. As a test antibody, Urelumab serially diluted from a maximum concentration of 5000 ng/ml with a common ratio of 2 or the anti-CLDN4 (hKM3900)/anti-CD137 scFv bispecific antibody (hKM3900_3-34-j6 or hKM3900_tA2-32LH) was used. Each of the test antibody and an isotype control (lysozyme antibody) was added in an amount of 20 μL each. After 4 days, a production amount of interferon γ in a supernatant was measured. FIG. 3-2 illustrates a reaction curve of the production amount of interferon γ and the antibody concentration. The anti-CLDN4 (hKM3900)/anti-CD137 scFv bispecific antibody exhibited interferon-γ production promoting function in a coculture system of the 60As6-Luc/GFP cells and the expanded Pan T cells. The interferon-γ production promoting function of these bispecific antibodies was remarkably stronger than that of Urelumab.

[6-5. Evaluation of Interferon-γ Production Promoting Function of Anti-CLDN4 (3D11)/anti-CD137 scFv Bispecific Antibody in Monoculture System of T Cell]

The interferon-γ production promoting function of the anti-CLDN4 (3D11)/anti-CD137 scFv bispecific antibody in a monoculture system of the expanded Pan T cells was evaluated. An anti-CD3 antibody was added to a flat bottom 96 well plate in an amount of 0.1 μg/100 μL/well to immobilize the anti-CD3 antibody on the plate. The expanded Pan T cells were seeded in the immobilized plate at 2×10⁵ cells/80 μL/well. As a test antibody, Urelumab and the anti-CLDN4 (3D11)/anti-CD3 scFv bispecific antibody (3D11_3-34-j6_S) were used, and as an isotype control, an IgG (lysozyme)-scFv (CD137) (prepared in house) was used. The test antibodies and the isotype control were serially diluted with the culture medium from a maximum concentration of 50000 ng/mL with a common ratio of 5 and added in an amount of 20 μL each. After 6 days, a production amount of interferon γ in a supernatant was measured with AlphaLISA Interferon-γ Measurement Kit in the same method as in 6-3. FIG. 3-3 illustrates a reaction curve of the production amount of interferon γ and the antibody concentration. The anti-CLDN4 (3D11)/anti-CD137 scFv bispecific antibody did not exhibit interferon-γ production promoting function in a monoculture system of a T cell, but Urelumab exhibited interferon-γ production promoting activity. This result means that the anti-CLDN4 (3D11)/anti-CD137 scFv bispecific antibody does not exhibit T cell activity enhancing activity in an environment where there is no CLDN4 expressing cancer cell line.

[6-6. Evaluation of Interferon-γ Production Promoting Function of Anti-CLDN4 (hKM3900)/anti-CD137 scFv Bispecific Antibody in Monoculture System of T Cell]

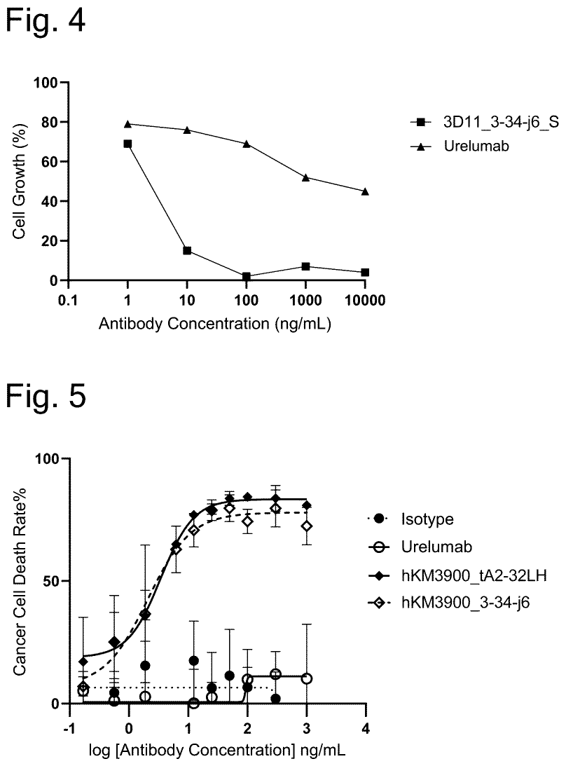

The interferon-γ production promoting function of the anti-CLDN4 (hKM3900)/anti-CD137 scFv bispecific antibody in a monoculture system of the expanded Pan T cells was evaluated in a method similar to that employed in 6-5. An anti-CD3 antibody was immobilized on a 96 well plate at 0.03 μg/100 μL/well. The expanded Pan T cells were seeded at 8×10⁴ cells/80 μL/well. As a test antibody, Urelumab and the anti-CLDN4 (hKM3900)/anti-CD137 scFv bispecific antibody (hKM3900_3-34-j6 or hKM3900_tA2-32 LH) were used. The test antibodies and an isotype control (lysozyme antibody) were serially diluted from a maximum concentration of 5000 ng/ml with a common ratio of 2 and added in an amount of 20 μL each. FIG. 3-4 illustrates a reaction curve of the interferon-γ production and the antibody concentration. The anti-CLDN4 (hKM3900)/anti-CD137 scFv bispecific antibody did not exhibit interferon-γ production promoting function in a monoculture system of a T cell, but Urelumab exhibited interferon-γ production promoting function.

The results in 6-5 and 6-6 mean that the anti-CLDN4/anti-CD137 scFv bispecific antibody does not exhibit T cell activity enhancing activity in an environment where there is no CLDN4 expressing cancer cell and suggest that systemic immunopotentiating activity reported as an adverse reaction of the anti-CD137 antibody is possibly reduced.

[6-7. Evaluation of Cancer Cytotoxic Activity of Anti-CLDN4 (3D11)/anti-CD137 scFv Bispecific Antibody in Coculture System of Cancer Cell and T Cell]

The cancer cytotoxic activity (cancer cell growth inhibitory activity) of the anti-CLDN4 (3D11)/anti-CD137 scFv bispecific antibody was evaluated in a coculture system of the 60As6-Luc/GFP cells and the expanded Pan T cells. The 60As6-Luc/GFP cells were seeded in a flat bottom 96 well plate at 1×10⁴ cells/50 μL/well each to be cultured at 37° C. in a 5% CO₂ incubator. After 2 hours, the expanded Pan T cells were seeded in the resultant 96 well plate at 1×10⁵ cells/50 μL/well each. As a test antibody, Urelumab and the anti-CLDN4 (3D11)/anti-CD137 scFv bispecific antibody were used. These antibodies were serially diluted with the culture medium from a maximum concentration of 40000 ng/mL with a common ratio of 10 and added in an amount of 50 μL each. Besides, a 40 ng/ml anti-CD3 antibody was added thereto in an amount of 50 μL each, and measurement of fluorescence (GFP) area in each well was initiated with IncuCyte® ZOOM (Sartorius) at 37° C. in a 5% CO₂ incubator. The fluorescence area increased from 0 hours after the start of the measurement was used as an index of cell growth. FIG. 4 illustrates cancer cytotoxic activity (cancer cell growth rate) of a test antibody. The ordinate of FIG. 4 indicates a relative value obtained assuming that, in fluorescence area increased from 0 hours to 168 hours, a well where the test antibody was not added corresponds to 100%. As illustrated in FIG. 4, the anti-CLDN4 (3D11)/anti-CD137 scFv bispecific antibody exhibited the cytotoxic activity in a coculture system of the 60As6-Luc/GFP cells and the expanded Pan T cells. The cytotoxic activity of this bispecific antibody was remarkably stronger than that of Urelumab.

[6-8. Evaluation of Cancer Cell Cytotoxic Activity of Anti-CLDN4 (hKM3900)/anti-CD137 scFv Bispecific Antibody in Coculture System of Cancer Cell and T Cell]

The cancer cell killing activity of the anti-CLDN4 (hKM3900)/anti-CD137 scFv bispecific antibody was evaluated in a coculture system of 60As6-Luc/GFP cells and the expanded Pan T cells. An anti-CD3 antibody was added to a flat bottom 96 well plate (Perkin Elmer, 6005680) at 1 ng/100 μL/well each to be fixed on the plate. The plate was washed with PBS (−) twice, the 60As6-Luc/GFP cells were seeded therein at 2×10⁴ cells/50 μL/well, and the resultant was cultured at 37° C. in a 5% CO₂ incubator. On the next day, the expanded Pan T cells were seeded in the 96 well plate at 4.0×10⁴ cells/30 μL/well. Besides, a test antibody and an isotype control at a concentration from 5000 ng/ml to 0.844 ng/ml were added in an amount of 20 μL each. As the test antibody, Urelumab and the anti-CLDN4 (hKM3900)/anti-CD137 scFv bispecific antibody (hKM3900_3-34-j6 or hKM3900_tA2-32 LH) were used. After 6 days, the measurement was performed. To some of the wells, 1% Triton-X100 (Sigma-Aldrich, T-9284) was added in an amount of 11 μL each for killing cells 30 minutes before the measurement. ONE-Glo Luciferase Measurement Kit (Promega Corporation, E6120) was used in accordance with a protocol recommended by the manufacturer to measure the luciferase activity of the 60As6-Luc/GFP cells in each well with ARVO X-3 (Perkin Elmer). A cancer death rate is indicated as a relative value assuming that an average of luciferase chemiluminescence in a well where the antibody was not added corresponds to 0% and that an average obtained in a well where Triton-X100 was added corresponds to 100%. As the isotype control, an anti-lysozyme antibody (prepared in house) was used. FIG. 5 illustrates a cell death rate curve of 60As6-Luc/GFP cells. In the coculture system of the 60As6-Luc/GFP cells and the expanded Pan T cells, the addition of the anti-CLDN4 (hKM3900)/anti-CD137 scFv bispecific antibody remarkably increased the 60As6-Luc/GFP cell death rate. On the other hand, Urelumab, the anti-CD137 specific antibody, did not increase the cell death rate of the 60As6-Luc/GFP cell line.

### Example 7: In Vivo Evaluation of Anti-CLDN4/Anti-CD137 scFv Bispecific Antibody

[7-1. In Vivo Antitumor Effect of Anti-CLDN4/Anti-CD137 scFv Bispecific Antibody]

A NCI-H322 cell was suspended in PBS (−) (WAKO, 045-29795), and a 5.0×10⁶ cells/100 μL cell suspension was subcutaneously inoculated into 7-week-old female mice (NOG, In-Vivo Science Inc.). After 6 days of the inoculation, 3.4×10⁶ cells/200 μL human peripheral blood mononuclear cells (Lonza, CC-2702) were injected through the tail vein. 15 days after the human peripheral blood mononuclear cell transplantation, the mice were divided into groups to start administration of the test antibody. Each group consisted of 10 mice, and the mice were distributed so that tumor volume between each group was almost equivalent to each other (day 0). As the test antibody, hKM3900_tA2-32 LH was used. The test antibody and an isotype control (anti-lysozyme antibody) were mixed in PBS and intravenously administered. The administration was performed on days 0, 4, and 8, and tumor diameter and body weight were measured at the time of administration. For calculation of tumor volume, the following formula was used.

$$[ Tumor volume ( {mm}^{3} ) ] = [ \text{} Major tumor diameter ( mm ) ] \times {[ Minor tumor diameter ( mm ) ]}^{2} \times 0.5$$

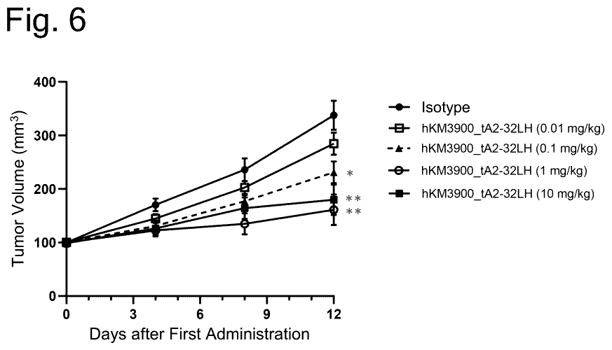

As illustrated in FIG. 6, a significant effect of reducing tumor volume was exhibited in the groups administered with 0.1, 1, or 10 mg/kg of hKM3900_tA2-32 LH. The ordinate indicates the tumor volume, and the abscissa indicates days after the first administration. The solid and dashed lines indicate a shift in an average of the tumor volume for each group, and an error bar indicates the standard error of the average. Significance probability P-values (*: P<0.05 and **: P<0.01) were calculated by comparing the tumor volume on day 12 in the isotype control administration group and the hKM3900_tA2-32 LH administration group through Dunnett's multiple comparison test. The number of cases in the 0.1 mg/kg hKM3900_tA2-32 LH administration group on day 12 was 9.

[7-2. Safety Test of Anti-CLDN4 (hKM3900)/anti-CD137 scFv Bispecific Antibody in Cynomolgus Monkey]

In order to examine safety of the anti-CLDN4 (hKM3900)/anti-CD137 scFv bispecific antibodies (hKM3900_3-34-j6 and hKM3900_tA2-32 LH), each of the anti-CLDN4 (hKM3900)/anti-CD137 scFv bispecific antibodies was administered in a single dose intravenously to cynomolgus monkeys. As a result, no specific abnormalities were observed in levels of cytokines such as IL-6 and TNF-α in the serum of the monkeys administered with the anti-CLDN4 (hKM3900)/anti-CD137 scFv bispecific antibodies. Hence, it was concluded that the anti-CLDN4 (hKM3900)/anti-CD137 scFv bispecific antibodies are qualified as antibodies for treatment of cancer from the viewpoint of safety.

### Examples 8: Treatment of Cancer by Anti-CLDN4/anti-CD137 Bispecific Antibody

### [8-1. Check of Expression Level of CLDN4]

In order to investigate how widely applicable CLDN4 is as a therapeutic target for cancer, the expression level of CLDN4 RNA in cancer tissues was investigated in The Cancer Genome Atlas (TCGA). As a result, it was confirmed that the expression of CLDN4 was elevated in cancer tissues of colorectal cancer, bladder cancer, lung cancer, and the like. Accordingly, it was suggested that CLDN4 is a possible therapeutic target not only for peritoneal metastatic cancer but also for other types of cancer. Besides, the expression level of CLDN4 RNA in normal tissues was investigated with the Genotype-Tissue Expression (GTEx). As a result, CLDN4 was found to be expressed in several normal tissues. However, the expression level of CLDN4 was lower in the normal tissues than in the cancer tissues. Therefore, it was suggested that a cancer drug therapeutically targeting CLDN4 is a possible therapeutic agent with a wide margin of safety ensured and a low concern for an adverse reaction.

### INDUSTRIAL APPLICABILITY

An anti-CLDN4/anti-CD137 bispecific antibody of the present invention is expected to be useful for treatment of cancer. Besides, a polynucleotide, an expression vector, a transformed host cell, and a method for producing an antibody of the present invention are useful for producing the anti-CLDN4/anti-CD137 bispecific antibody.

### SEQUENCE LISTING FREE TEXT

In number heading <223> of the following sequence listing, “Artificial Sequence” is described. Specifically, SEQ ID NO: 1 of the sequence listing shows a nucleotide sequence of a heavy chain variable region of 3D11, and SEQ ID NO: 2 shows an amino acid sequence of the heavy chain variable region of 3D11. SEQ ID NO: 3 shows a nucleotide sequence of a light chain variable region of 3D11, and SEQ ID NO: 4 shows an amino acid sequence of the light chain variable region of 3D11. SEQ ID NO: 5 shows a nucleotide sequence of a heavy chain variable region of hKM3900, and SEQ ID NO: 6 shows an amino acid sequence of the heavy chain variable region of hKM3900. SEQ ID NO: 7 shows a nucleotide sequence of a light chain variable region of hKM3900, and SEQ ID NO: 8 shows an amino acid sequence of the light chain variable region of hKM3900. SEQ ID NO: 9 shows a nucleotide sequence of a heavy chain variable region of 3-34, and SEQ ID NO: 10 shows an amino acid sequence of the heavy chain variable region of 3-34. SEQ ID NO: 11 shows a nucleotide sequence of a light chain variable region of 3-34, and SEQ ID NO: 12 shows an amino acid sequence of the light chain variable region of 3-34. SEQ ID NO: 13 shows a nucleotide sequence of a heavy chain variable region of A2-32, and SEQ ID NO: 14 shows an amino acid sequence of the heavy chain variable region of A2-32. SEQ ID NO: 15 shows a nucleotide sequence of a light chain variable region of A2-32, and SEQ ID NO: 16 shows an amino acid sequence of the light chain variable region of A2-32. SEQ ID NO: 17 shows a nucleotide sequence of a heavy chain variable region of A2-48, and SEQ ID NO: 18 shows an amino acid sequence of the heavy chain variable region of A2-48. SEQ ID NO: 19 shows a nucleotide sequence of a light chain variable region of A2-48, and SEQ ID NO: 20 shows an amino acid sequence of the light chain variable region of A2-48. SEQ ID NO: 21 shows a nucleotide sequence of a heavy chain variable region of A2-73, and SEQ ID NO: 22 shows an amino acid sequence of the heavy chain variable region of A2-73. SEQ ID NO: 23 shows a nucleotide sequence of a light chain variable region of A2-73, and SEQ ID NO: 24 shows an amino acid sequence of the light chain variable region of A2-73. SEQ ID NO: 25 shows a nucleotide sequence of a 3-34-j6 scFv, and SEQ ID NO: 26 shows an amino acid sequence of the 3-34-j6 scFv. SEQ ID NO: 27 shows a nucleotide sequence of a tA2-32 scFv, and SEQ ID NO: 28 shows an amino acid sequence of the tA2-32 scFv. SEQ ID NO: 29 shows a nucleotide sequence of a tA2-32 LH scFv, and SEQ ID NO: 30 shows an amino acid sequence of the tA2-32 LH scFv. SEQ ID NO: 31 shows a nucleotide sequence of a tA2-48 scFv, and SEQ ID NO: 32 shows an amino acid sequence of the tA2-48 scFv. SEQ ID NO: 33 shows a nucleotide sequence of a tA2-73 scFv, and SEQ ID NO: 34 shows an amino acid sequence of the tA2-73 scFv. SEQ ID NO: 35 shows a nucleotide sequence of a heavy chain of hKM3900_3-34-j6, and SEQ ID NO: 36 shows an amino acid sequence of the heavy chain of hKM3900_3-34-j6. SEQ ID NO: 37 shows a nucleotide sequence of a heavy chain of hKM3900_tA2-32 LH, and SEQ ID NO: 38 shows an amino acid sequence of the heavy chain of hKM3900_tA2-32 LH. SEQ ID NO: 39 shows a nucleotide sequence of a light chain of hKM3900, and SEQ ID NO: 40 shows an amino acid sequence of the light chain of hKM3900. SEQ ID NO: 41 shows an amino acid sequence of an anti-CD3 scFv-human Fc fusion protein. SEQ ID NO: 42 shows an amino acid sequence of human CD137. SEQ ID NO: 43 shows an amino acid sequence of monkey CD137. SEQ ID NO: 44 shows an amino acid sequence of a His tag protein. SEQ ID NO: 45 shows an amino acid sequence of a 3×FLAG tag. SEQ ID NOS: 46 to 54 show amino acid sequences of GS linkers. SEQ ID NO: 55 shows an amino acid sequence of a GKPGS linker.

What is claimed is:

1. An anti-CLDN4/anti-CD137 bispecific antibody comprising a heavy chain variable region and a light chain variable region of an anti-CLDN4 antibody, and a heavy chain variable region and a light chain variable region of an anti-CD137 antibody, wherein the heavy chain variable region and the light chain variable region of the anti-CLDN4 antibody are either of the following (a) or (b):
(a) a heavy chain variable region containing a CDR1 consisting of an amino acid sequence at amino acid positions 31-35 of SEQ ID NO: 2, a CDR2 consisting of an amino acid sequence at amino acid positions 50-66 of SEQ ID NO: 2, and a CDR3 consisting of an amino acid sequence at amino acid positions 99-114 of SEQ ID NO: 2, and a light chain variable region of an anti-CLDN4 antibody containing a CDR1 consisting of an amino acid sequence at amino acid positions 24-35 of SEQ ID NO: 4, a CDR2 consisting of an amino acid sequence at amino acid positions 51-57 of SEQ ID NO: 4, and a CDR3 consisting of an amino acid sequence at amino acid positions 90-98 of SEQ ID NO: 4; or
(b) a heavy chain variable region containing a CDR1 consisting of an amino acid sequence at amino acid positions 31-35 of SEQ ID NO: 6, a CDR2 consisting of an amino acid sequence at amino acid positions 50-66 of SEQ ID NO: 6, and a CDR3 consisting of an amino acid sequence at amino acid positions 99-112 of SEQ ID NO: 6, and a light chain variable region containing a CDR1 consisting of an amino acid sequence at amino acid positions 24-35 of SEQ ID NO: 8, a CDR2 consisting of an amino acid sequence at amino acid positions 51-57 of SEQ ID NO: 8, and a CDR3 consisting of an amino acid sequence at amino acid positions 90-98 of SEQ ID NO: 8.

2. The bispecific antibody according to claim 1, wherein the heavy chain variable region and the light chain variable region of the anti-CLDN4 antibody are either of the following (a) or (b):
(a) a heavy chain variable region consisting of an amino acid sequence at amino acid positions 1-125 of SEQ ID NO: 2 and a light chain variable region consisting of an amino acid sequence at amino acid positions 1-109 of SEQ ID NO: 4; or
(b) a heavy chain variable region consisting of an amino acid sequence at amino acid positions 1-123 of SEQ ID NO: 6 and a light chain variable region consisting of an amino acid sequence at amino acid positions 1-109 of SEQ ID NO: 8.

3. The bispecific antibody according to claim 1, comprising an IgG antibody consisting of a heavy chain containing the heavy chain variable region and a light chain containing the light chain variable region of the anti-CLDN4 antibody (anti-CLDN4 IgG antibody).

4. The bispecific antibody according to claim 3, comprising LALA mutation (L234A and L235A, wherein a position of the mutation is an amino acid position in human Igγ1 constant region according to EU index) in a Fc region of the anti-CLDN4 IgG antibody.

5. The bispecific antibody according to claim 3, comprising P331G mutation in a Fc region of the anti-CLDN4 IgG antibody (wherein a position of the mutation is an amino acid position in human Igγ1 constant region according to EU index).

6. The bispecific antibody according to claim 3, comprising LALA mutation and P331G mutation in a Fc region of the anti-CLDN4 IgG antibody.

7. The bispecific antibody according to claim 1, wherein the heavy chain variable region and the light chain variable region of the anti-CD137 antibody are any of the following (a) to (d):
(a) a heavy chain variable region containing a CDR1 consisting of an amino acid sequence at amino acid positions 31-35 of SEQ ID NO: 10, a CDR2 consisting of an amino acid sequence at amino acid positions 50-66 of SEQ ID NO: 10, and a CDR3 consisting of an amino acid sequence at amino acid positions 99-107 of SEQ ID NO: 10, and a light chain variable region containing a CDR1 consisting of an amino acid sequence at amino acid positions 24-34 of SEQ ID NO: 12, a CDR2 consisting of an amino acid sequence at amino acid positions 50-56 of SEQ ID NO: 12, and a CDR3 consisting of an amino acid sequence at amino acid positions 89-98 of SEQ ID NO: 12;
(b) a heavy chain variable region containing a CDR1 consisting of an amino acid sequence at amino acid positions 31-35 of SEQ ID NO: 14, a CDR2 consisting of an amino acid sequence at amino acid positions 50-65 of SEQ ID NO: 14, and a CDR3 consisting of an amino acid sequence at amino acid positions 98-107 of SEQ ID NO: 14, and a light chain variable region containing a CDR1 consisting of an amino acid sequence at amino acid positions 23-35 of SEQ ID NO: 16, a CDR2 consisting of an amino acid sequence at amino acid positions 51-57 of SEQ ID NO: 16, and a CDR3 consisting of an amino acid sequence at amino acid positions 90-100 of SEQ ID NO: 16;
(c) a heavy chain variable region containing a CDR1 consisting of an amino acid sequence at amino acid positions 31-35 of SEQ ID NO: 18, a CDR2 consisting of an amino acid sequence at amino acid positions 50-65 of SEQ ID NO: 18, and a CDR3 consisting of an amino acid sequence at amino acid positions 98-107 of SEQ ID NO: 18, and a light chain variable region containing a CDR1 consisting of an amino acid sequence at amino acid positions 23-35 of SEQ ID NO: 20, a CDR2 consisting of an amino acid sequence at amino acid positions 51-57 of SEQ ID NO: 20, and a CDR3 consisting of an amino acid sequence at amino acid positions 90-100 of SEQ ID NO: 20; or
(d) a heavy chain variable region containing a CDR1 consisting of an amino acid sequence at amino acid positions 31-35 of SEQ ID NO: 22, a CDR2 consisting of an amino acid sequence at amino acid positions 50-65 of SEQ ID NO: 22, and a CDR3 consisting of an amino acid sequence at amino acid positions 98-110 of SEQ ID NO: 22, and a light chain variable region containing a CDR1 consisting of an amino acid sequence at amino acid positions 23-35 of SEQ ID NO: 24, a CDR2 consisting of an amino acid sequence at amino acid positions 51-57 of SEQ ID NO: 24, and a CDR3 consisting of an amino acid sequence at amino acid positions 90-100 of SEQ ID NO: 24.

8. The bispecific antibody according to claim 2, wherein the heavy chain variable region and the light chain variable region of the anti-CD137 antibody are any of the following (a) to (i):
(a) a heavy chain variable region consisting of an amino acid sequence at amino acid positions 1-118 of SEQ ID NO: 10 and a light chain variable region consisting of an amino acid sequence at amino acid positions 1-109 of SEQ ID NO: 12;
(b) a heavy chain variable region consisting of an amino acid sequence at amino acid positions 1-118 of SEQ ID NO: 14 and a light chain variable region consisting of an amino acid sequence at amino acid positions 1-111 of SEQ ID NO: 16;
(c) a heavy chain variable region consisting of an amino acid sequence at amino acid positions 1-118 of SEQ ID NO: 18 and a light chain variable region consisting of an amino acid sequence at amino acid positions 1-111 of SEQ ID NO: 20;
(d) a heavy chain variable region consisting of an amino acid sequence at amino acid positions 1-121 of SEQ ID NO: 22 and a light chain variable region consisting of an amino acid sequence at amino acid positions 1-111 of SEQ ID NO: 24;
(e) a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26;
(f) a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28;
(g) a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30;
(h) a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32; or
(i) a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34.

9. The bispecific antibody according to claim 3, comprising an anti-CD137 single chain variable fragment (anti-CD137 scFv) containing the heavy chain variable region and the light chain variable region of the anti-CD137 antibody.

10. The bispecific antibody according to claim 9, wherein the anti-CD137 scFv is any of the following (a) to (e):
(a) an anti-CD137 scFv consisting of an amino acid sequence at amino acid positions 1-242 of SEQ ID NO: 26;
(b) an anti-CD137 scFv consisting of an amino acid sequence at amino acid positions 1-244 of SEQ ID NO: 28;
(c) an anti-CD137 scFv consisting of an amino acid sequence at amino acid positions 1-249 of SEQ ID NO: 30;
(d) an anti-CD137 scFv consisting of an amino acid sequence at amino acid positions 1-244 of SEQ ID NO: 32; or
(e) an anti-CD137 scFv consisting of an amino acid sequence at amino acid positions 1-247 of SEQ ID NO: 34.

11. The bispecific antibody according to claim 10, comprising an anti-CLDN4 IgG antibody and the anti-CD137 scFv, wherein an amino terminal of the anti-CD137 scFv is linked to a heavy chain or light chain carboxy terminal of the anti-CLDN4 IgG antibody via a linker.

12. An anti-CLDN4/anti-CD137 bispecific antibody described in any of the following (a) to (j):
(a) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26, wherein an amino terminal of the anti-CD137 scFv is linked to a heavy chain carboxy terminal of the anti-CLDN4 antibody via a linker;
(b) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28, wherein an amino terminal of the anti-CD137 scFv is linked to a heavy chain carboxy terminal of the anti-CLDN4 antibody via a linker;
(c) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30, wherein an amino terminal of the anti-CD137 scFv is linked to a heavy chain carboxy terminal of the anti-CLDN4 antibody via a linker;
(d) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32, wherein an amino terminal of the anti-CD137 scFv is linked to a heavy chain carboxy terminal of the anti-CLDN4 antibody via a linker;
(e) a bispecific antibody comprising a heavy chain of an anti-CLDN4 antibody containing a heavy chain variable region consisting of an amino acid sequence at amino acid positions 1-125 of SEQ ID NO: 2 and a light chain of an anti-CLDN4 antibody containing a light chain variable region consisting of an amino acid sequence at amino acid positions 1-109 of SEQ ID NO: 4, and an anti-CD137 scFv containing a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34, wherein an amino terminal of the anti-CD137 scFv is linked to a heavy chain carboxy terminal of the anti-CLDN4 antibody via a linker;
(f) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 26 and a light chain variable region consisting of an amino acid sequence at amino acid positions 134-242 of SEQ ID NO: 26, wherein an amino terminal of the anti-CD137 scFv is linked to a heavy chain carboxy terminal of the anti-CLDN4 antibody via a linker;
(g) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 28 and a light chain variable region consisting of an amino acid sequence at amino acid positions 134-244 of SEQ ID NO: 28, wherein an amino terminal of the anti-CD137 scFv is linked to a heavy chain carboxy terminal of the anti-CLDN4 antibody via a linker;
(h) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a light chain variable region consisting of an amino acid sequence at amino acid positions 1-111 of SEQ ID NO: 30 and a heavy chain variable region consisting of an amino acid sequence at amino acid positions 132-249 of SEQ ID NO: 30, wherein an amino terminal of the anti-CD137 scFv is linked to a heavy chain carboxy terminal of the anti-CLDN4 antibody via a linker;
(i) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-118 of SEQ ID NO: 32 and a light chain variable region consisting of an amino acid sequence at amino acid positions 134-244 of SEQ ID NO: 32, wherein an amino terminal of the anti-CD137 scFv is linked to a heavy chain carboxy terminal of the anti-CLDN4 antibody via a linker; or
(j) a bispecific antibody comprising a heavy chain of an anti-CLDN4 antibody containing a heavy chain variable region consisting of an amino acid sequence at amino acid positions 1-123 of SEQ ID NO: 6 and a light chain of an anti-CLDN4 antibody containing a light chain variable region consisting of an amino acid sequence at amino acid positions 1-109 of SEQ ID NO: 8, and an anti-CD137 scFv containing a heavy chain variable region consisting of an amino acid sequence at amino acid positions 1-121 of SEQ ID NO: 34 and a light chain variable region consisting of an amino acid sequence at amino acid positions 137-247 of SEQ ID NO: 34, wherein an amino terminal of the anti-CD137 scFv is linked to a heavy chain carboxy terminal of the anti-CLDN4 antibody via a linker.

13. The anti-CLDN4/anti-CD137 bispecific antibody according to claim 12, wherein the linker is a GS linker.

14. The anti-CLDN4/anti-CD137 bispecific antibody according to claim 13, wherein the GS linker is a linker consisting of an amino acid sequence of SEQ ID NO: 54.

15. An anti-CLDN4/anti-CD137 bispecific antibody described in either of the following (a) or (b):
(a) a bispecific antibody comprising a polypeptide consisting of an amino acid sequence at amino acid positions 1-705 of SEQ ID NO: 36 containing a heavy chain of an anti-CLDN4 antibody and an anti-CD137 scFv, and a light chain of an anti-CLDN4 antibody consisting of an amino acid sequence at amino acid positions 1-215 of SEQ ID NO: 40; or
(b) a bispecific antibody comprising a polypeptide consisting of an amino acid sequence at amino acid positions 1-712 of SEQ ID NO: 38 containing a heavy chain of an anti-CLDN4 antibody and an anti-CD137 scFv, and a light chain of an anti-CLDN4 antibody consisting of an amino acid sequence at amino acid positions 1-215 of SEQ ID NO: 40.

16. The anti-CLDN4/anti-CD137 bispecific antibody according to claim 15, which is post-translationally modified.

17. The anti-CLDN4/anti-CD137 bispecific antibody according to claim 16, wherein the post-translational modification is pyroglutamylation at an N-terminal of a heavy chain variable region and/or lysine deletion at a heavy chain C-terminal.

18. A polynucleotide having a nucleotide sequence encoding a heavy chain variable region or light chain variable region of an anti-CLDN4 antibody, which is selected from the group consisting of the following (a) to (d):
(a) a polynucleotide having a nucleotide sequence encoding a heavy chain variable region of an anti-CLDN4 antibody consisting of an amino acid sequence at amino acid positions 1-125 of SEQ ID NO: 2;
(b) a polynucleotide having a nucleotide sequence encoding a light chain variable region of an anti-CLDN4 antibody consisting of an amino acid sequence at amino acid positions 1-109 of SEQ ID NO: 4;
(c) a polynucleotide having a nucleotide sequence encoding a heavy chain variable region of an anti-CLDN4 antibody consisting of an amino acid sequence at amino acid positions 1-123 of SEQ ID NO: 6; or
(d) a polynucleotide having a nucleotide sequence encoding a light chain variable region of an anti-CLDN4 antibody consisting of an amino acid sequence at amino acid positions 1-109 of SEQ ID NO: 8.

19. A polynucleotide having a nucleotide sequence encoding a heavy chain variable region or light chain variable region of an anti-CD137 antibody, which is selected from the group consisting of the following (a) to (r):
(a) a polynucleotide having a nucleotide sequence encoding a heavy chain variable region of an anti-CD137 antibody consisting of an amino acid sequence at amino acid positions 1-118 of SEQ ID NO: 10;
(b) a polynucleotide having a nucleotide sequence encoding a light chain variable region of an anti-CD137 antibody consisting of an amino acid sequence at amino acid positions 1-109 of SEQ ID NO: 12;
(c) a polynucleotide having a nucleotide sequence encoding a heavy chain variable region of an anti-CD137 antibody consisting of an amino acid sequence at amino acid positions 1-118 of SEQ ID NO: 14;
(d) a polynucleotide having a nucleotide sequence encoding a light chain variable region of an anti-CD137 antibody consisting of an amino acid sequence at amino acid positions 1-111 of SEQ ID NO: 16;
(e) a polynucleotide having a nucleotide sequence encoding a heavy chain variable region of an anti-CD137 antibody consisting of an amino acid sequence at amino acid positions 1-118 of SEQ ID NO: 18;
(f) a polynucleotide having a nucleotide sequence encoding a light chain variable region of an anti-CD137 antibody consisting of an amino acid sequence at amino acid positions 1-111 of SEQ ID NO: 20;
(g) a polynucleotide having a nucleotide sequence encoding a heavy chain variable region of an anti-CD137 antibody consisting of an amino acid sequence at amino acid positions 1-121 of SEQ ID NO: 22;
(h) a polynucleotide having a nucleotide sequence encoding a light chain variable region of an anti-CD137 antibody consisting of an amino acid sequence at amino acid positions 1-111 of SEQ ID NO: 24;
(i) a polynucleotide having a nucleotide sequence encoding a heavy chain variable region of an anti-CD137 antibody consisting of an amino acid sequence at amino acid positions 1-118 of SEQ ID NO: 26;
(j) a polynucleotide having a nucleotide sequence encoding a light chain variable region of an anti-CD137 antibody consisting of an amino acid sequence at amino acid positions 134-242 of SEQ ID NO: 26;
(k) a polynucleotide having a nucleotide sequence encoding a heavy chain variable region of an anti-CD137 antibody consisting of an amino acid sequence at amino acid positions 1-118 of SEQ ID NO: 28;
(l) a polynucleotide having a nucleotide sequence encoding a light chain variable region of an anti-CD137 antibody consisting of an amino acid sequence at amino acid positions 134-244 of SEQ ID NO: 28;
(m) a polynucleotide having a nucleotide sequence encoding a light chain variable region of an anti-CD137 antibody consisting of an amino acid sequence at amino acid positions 1-111 of SEQ ID NO: 30;
(n) a polynucleotide having a nucleotide sequence encoding a heavy chain variable region of an anti-CD137 antibody consisting of an amino acid sequence at amino acid positions 132-249 of SEQ ID NO: 30;
(o) a polynucleotide having a nucleotide sequence encoding a heavy chain variable region of an anti-CD137 antibody consisting of an amino acid sequence at amino acid positions 1-118 of SEQ ID NO: 32;
(p) a polynucleotide having a nucleotide sequence encoding a light chain variable region of an anti-CD137 antibody consisting of an amino acid sequence at amino acid positions 134-244 of SEQ ID NO: 32;
(q) a polynucleotide having a nucleotide sequence encoding a heavy chain variable region of an anti-CD137 antibody consisting of an amino acid sequence at amino acid positions 1-121 of SEQ ID NO: 34; or
(r) a polynucleotide having a nucleotide sequence encoding a light chain variable region of an anti-CD137 antibody consisting of an amino acid sequence at amino acid positions 137-247 of SEQ ID NO: 34.

20. A polynucleotide having a nucleotide sequence encoding an anti-CD137 scFv, which is selected from the group consisting of the following (a) to (e):
(a) a polynucleotide having a nucleotide sequence encoding an anti-CD137 scFv consisting of an amino acid sequence at amino acid positions 1-242 of SEQ ID NO: 26;
(b) a polynucleotide having a nucleotide sequence encoding an anti-CD137 scFv consisting of an amino acid sequence at amino acid positions 1-244 of SEQ ID NO: 28;
(c) a polynucleotide having a nucleotide sequence encoding an anti-CD137 scFv consisting of an amino acid sequence at amino acid positions 1-249 of SEQ ID NO: 30;
(d) a polynucleotide having a nucleotide sequence encoding of an anti-CD137 scFv consisting of an amino acid sequence at amino acid positions 1-244 of SEQ ID NO: 32; or
(e) a polynucleotide having a nucleotide sequence encoding an anti-CD137 scFv consisting of an amino acid sequence at amino acid positions 1-247 of SEQ ID NO: 34.

21. A polynucleotide for use in production of the anti-CLDN4/anti-CD137 bispecific antibody according to claim 15, which is selected from the group consisting of the following (a) to (e):
(a) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv;
(b) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv;
(c) a polynucleotide having a nucleotide sequence encoding a light chain of an anti-CLDN4 antibody consisting of an amino acid sequence at amino acid positions 1-215 of SEQ ID NO: 40;
(d) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40; or
(e) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40.

22. An expression vector, comprising the polynucleotide according to claim 18.

23. A host cell transformed with the expression vector according to claim 22.

24. A host cell comprising a polynucleotide selected from the group consisting of the following (a) to (dd):
(a) a polynucleotide having a nucleotide sequence encoding a heavy chain variable region of an anti-CLDN4 antibody consisting of an amino acid sequence at amino acid positions 1-125 of SEQ ID NO: 2;
(b) a polynucleotide having a nucleotide sequence encoding a light chain variable region of an anti-CLDN4 antibody consisting of an amino acid sequence at amino acid positions 1-109 of SEQ ID NO: 4;
(c) a polynucleotide having a nucleotide sequence encoding a heavy chain variable region of an anti-CLDN4 antibody consisting of an amino acid sequence at amino acid positions 1-123 of SEQ ID NO: 6;
(d) a polynucleotide having a nucleotide sequence encoding a light chain variable region of an anti-CLDN4 antibody consisting of an amino acid sequence at amino acid positions 1-109 of SEQ ID NO: 8;
(e) a polynucleotide having a nucleotide sequence encoding a heavy chain variable region of an anti-CD137 antibody consisting of an amino acid sequence at amino acid positions 1-118 of SEQ ID NO: 10;
(f) a polynucleotide having a nucleotide sequence encoding a light chain variable region of an anti-CD137 antibody consisting of an amino acid sequence at amino acid positions 1-109 of SEQ ID NO: 12;
(g) a polynucleotide having a nucleotide sequence encoding a heavy chain variable region of an anti-CD137 antibody consisting of an amino acid sequence at amino acid positions 1-118 of SEQ ID NO: 14;
(h) a polynucleotide having a nucleotide sequence encoding a light chain variable region of an anti-CD137 antibody consisting of an amino acid sequence at amino acid positions 1-111 of SEQ ID NO: 16;
(i) a polynucleotide having a nucleotide sequence encoding a heavy chain variable region of an anti-CD137 antibody consisting of an amino acid sequence at amino acid positions 1-118 of SEQ ID NO: 18;
(j) a polynucleotide having a nucleotide sequence encoding a light chain variable region of an anti-CD137 antibody consisting of an amino acid sequence at amino acid positions 1-111 of SEQ ID NO: 20;
(k) a polynucleotide having a nucleotide sequence encoding a heavy chain variable region of an anti-CD137 antibody consisting of an amino acid sequence at amino acid positions 1-121 of SEQ ID NO: 22;
(1) a polynucleotide having a nucleotide sequence encoding a light chain variable region of an anti-CD137 antibody consisting of an amino acid sequence at amino acid positions 1-111 of SEQ ID NO: 24;
(m) a polynucleotide having a nucleotide sequence encoding a heavy chain variable region of an anti-CD137 antibody consisting of an amino acid sequence at amino acid positions 1-118 of SEQ ID NO: 26;
(n) a polynucleotide having a nucleotide sequence encoding a light chain variable region of an anti-CD137 antibody consisting of an amino acid sequence at amino acid positions 134-242 of SEQ ID NO: 26;
(o) a polynucleotide having a nucleotide sequence encoding a heavy chain variable region of an anti-CD137 antibody consisting of an amino acid sequence at amino acid positions 1-118 of SEQ ID NO: 28;
(p) a polynucleotide having a nucleotide sequence encoding a light chain variable region of an anti-CD137 antibody consisting of an amino acid sequence at amino acid positions 134-244 of SEQ ID NO: 28;
(q) a polynucleotide having a nucleotide sequence encoding a light chain variable region of an anti-CD137 antibody consisting of an amino acid sequence at amino acid positions 1-111 of SEQ ID NO: 30;
(r) a polynucleotide having a nucleotide sequence encoding a heavy chain variable region of an anti-CD137 antibody consisting of an amino acid sequence at amino acid positions 132-249 of SEQ ID NO: 30;
(s) a polynucleotide having a nucleotide sequence encoding a heavy chain variable region of an anti-CD137 antibody consisting of an amino acid sequence at amino acid positions 1-118 of SEQ ID NO: 32;
(t) a polynucleotide having a nucleotide sequence encoding a light chain variable region of an anti-CD137 antibody consisting of an amino acid sequence at amino acid positions 134-244 of SEQ ID NO: 32;
(u) a polynucleotide having a nucleotide sequence encoding a heavy chain variable region of an anti-CD137 antibody consisting of an amino acid sequence at amino acid positions 1-121 of SEQ ID NO: 34;
(v) a polynucleotide having a nucleotide sequence encoding a light chain variable region of an anti-CD137 antibody consisting of an amino acid sequence at amino acid positions 137-247 of SEQ ID NO: 34;
(w) a polynucleotide having a nucleotide sequence encoding an anti-CD137 scFv consisting of an amino acid sequence at amino acid positions 1-242 of SEQ ID NO: 26;
(x) a polynucleotide having a nucleotide sequence encoding an anti-CD137 scFv consisting of an amino acid sequence at amino acid positions 1-244 of SEQ ID NO: 28;
(y) a polynucleotide having a nucleotide sequence encoding an anti-CD137 scFv consisting of an amino acid sequence at amino acid positions 1-249 of SEQ ID NO: 30;
(z) a polynucleotide having a nucleotide sequence encoding an anti-CD137 scFv consisting of an amino acid sequence at amino acid positions 1-244 of SEQ ID NO: 32;
(aa) a polynucleotide having a nucleotide sequence encoding an anti-CD137 scFv consisting of an amino acid sequence at amino acid positions 1-247 of SEQ ID NO: 34;
(bb) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv;
(cc) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv; or
(dd) a polynucleotide having a nucleotide sequence encoding a light chain of an anti-CLDN4 antibody consisting of an amino acid sequence at amino acid positions 1-215 of SEQ ID NO: 40.

25. A host cell comprising a polynucleotide of either of the following (a) or (b):
(a) a polynucleotide having a nucleotide sequence encoding a polypeptide consisting of an amino acid sequence at amino acid positions 1-705 of SEQ ID NO: 36 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40; or
(b) a polynucleotide having a nucleotide sequence encoding a polypeptide consisting of an amino acid sequence at amino acid positions 1-712 of SEQ ID NO: 38 containing a heavy chain of an anti-CLDN4 antibody and an anti-CD137 scFv, and a polynucleotide having a nucleotide sequence encoding a light chain of an anti-CLDN4 antibody consisting of an amino acid sequence at amino acid positions 1-215 of SEQ ID NO: 40.

26. A method for producing an anti-CLDN4/anti-CD137 bispecific antibody, comprising a step of culturing the host cell according to claim 24.

27. A pharmaceutical composition comprising the anti-CLDN4/anti-CD137 bispecific antibody according to claim 1, and a pharmaceutically acceptable excipient.

28. The anti-CLDN4/anti-CD137 bispecific antibody according to claim 1 for use in treatment of cancer.

29. The pharmaceutical composition according to claim 27 for use in treatment of cancer.

30. A method for treating cancer, comprising a step of administering a therapeutically effective amount of the anti-CLDN4/anti-CD137 bispecific antibody according to claim 1 to a subject.


SEQUENCE LISTING (60 sequences)

SEQ ID NO: 1 - DNA, 375 bp, synthetic construct
  misc_feature 1..375: note = Description of Artificial Sequence: Synthetic polynucleotide
  misc_feature 1..375: note = 3D11 VH
  source 1..375: mol_type = other DNA
  CDS 1..375
caggttcagttgggacagtctggccccgaagtgaagaaacctggcgcctctgtgaaggtgtcctgcaaggcttccggctactcctttatctcctacggcatctcctgggtccgacaggctcctggacaaggcttggaatggatgggctggtcctccgtgtacaacggcaacaccaactacgtgcagaaattccagggcagagtgaccatgaccaccgacacctctgcctccaccgcctacatggaactgcggtccctgagatctgatgacgccgccgtgtactactgcgccagagactcttactcctccggctggtcttactactactccggcatggatgtgtggggccagggcacaacagtgaccgtgtcctcc

SEQ ID NO: 2 - protein, 125 aa, synthetic construct
  REGION 1..125: note = Description of Artificial Sequence: Synthetic polypeptide
  REGION 1..125: note = Synthetic Construct
QVQLGQSGPEVKKPGASVKVSCKASGYSFISYGISWVRQAPGQGLEWMGWSSVYNGNTNYVQKFQGRVTMTTDTSASTAYMELRSLRSDDAAVYYCARDSYSSGWSYYYSGMDVWGQGTTVTVSS

SEQ ID NO: 3 - DNA, 327 bp, synthetic construct
  misc_feature 1..327: note = Description of Artificial Sequence: Synthetic polynucleotide
  misc_feature 1..327: note = 3D11 VL
  source 1..327: mol_type = other DNA
  CDS 1..327
gagattgtgctgacccagtctcctggcacactgtctttgagccctggcgagagagctaccctgtcctgtagagcctctcagtccgtgtcctcctcttacctggcctggtatcagcagaagcctggacaggctccccggctgttgatctacggcgcttcttctagagccacaggcatccctgaccggttctccggatctggctctggcaccgatttcaccctgaccatcaaccggctggaacccgaggatttcgccgtgtactactgccagcagtacggctcctctccttactcttttggccagggcaccaagctggaaatcaagcgg

SEQ ID NO: 4 - protein, 109 aa, synthetic construct
  REGION 1..109: note = Description of Artificial Sequence: Synthetic polypeptide
  REGION 1..109: note = Synthetic Construct
EIVLTQSPGTLSLSPGERATLSCRASQSVSSSYLAWYQQKPGQAPRLLIYGASSRATGIPDRFSGSGSGTDFTLTINRLEPEDFAVYYCQQYGSSPYSFGQGTKLEIKR

SEQ ID NO: 5 - DNA, 369 bp, synthetic construct
  misc_feature 1..369: note = Description of Artificial Sequence: Synthetic polynucleotide
  misc_feature 1..369: note = hKM3900 VH
  source 1..369: mol_type = other DNA
  CDS 1..369
gaagtgcagctggttcagtctggcgccgaagtgaagaaacctggcgcctccgtgaagatctcctgcaaggcttctggctacaccttcaccgactactacatgaactgggtccgacaggctcctggacagggacttgagtggatgggagatgtggtgcccaacaacggcgtgcccacctacaaccagaaattcaagggcagagtgaccatcaccgccgacaagtctacctccaccgcctacatggaactgcggagcctgagatctgaggacaccgccgtgtactactgcgccagacctcactactactacgccggcagatctggcgccatggattattggggacagggcaccctggtcaccgtgtcctct

SEQ ID NO: 6 - protein, 123 aa, synthetic construct
  REGION 1..123: note = Description of Artificial Sequence: Synthetic polypeptide
  REGION 1..123: note = Synthetic Construct
EVQLVQSGAEVKKPGASVKISCKASGYTFTDYYMNWVRQAPGQGLEWMGDVVPNNGVPTYNQKFKGRVTITADKSTSTAYMELRSLRSEDTAVYYCARPHYYYAGRSGAMDYWGQGTLVTVSS

SEQ ID NO: 7 - DNA, 327 bp, synthetic construct
  misc_feature 1..327: note = Description of Artificial Sequence: Synthetic polynucleotide
  misc_feature 1..327: note = hKM3900 VL
  source 1..327: mol_type = other DNA
  CDS 1..327
gacatccagctgacccagtctccatcctctctgtctgcctctgtgggcgacagagtgacaattacctgcaccgcctcctccaccgtgtcctctacctacctgcactggtatcagcagaagcccggcaaggctcccaagctgctgatctactccacctccaatctggcctctggcgtgccctctagattctccggatctggctctggaaccgactataccctgacaatctccagcctgcagcctgaggacttcgccacctactactgccaccagtaccacagatccccacctacctttggccagggcaccaagctggaaatcaagcgt

SEQ ID NO: 8 - protein, 109 aa, synthetic construct
  REGION 1..109: note = Description of Artificial Sequence: Synthetic polypeptide
  REGION 1..109: note = Synthetic Construct
DIQLTQSPSSLSASVGDRVTITCTASSTVSSTYLHWYQQKPGKAPKLLIYSTSNLASGVPSRFSGSGSGTDYTLTISSLQPEDFATYYCHQYHRSPPTFGQGTKLEIKR

SEQ ID NO: 9 - DNA, 354 bp, synthetic construct
  misc_feature 1..354: note = Description of Artificial Sequence: Synthetic polynucleotide
  misc_feature 1..354: note = 3-34 VH
  source 1..354: mol_type = other DNA
  CDS 1..354
caggtccagctgcagcagtctgaggctgagctggtgaggcctggggcctcagtgaagatttcctgcaaggcttttggctacaccttcacaaaccatcatataaactgggtgaagcagaggcctggacagggcctggactggattggatatattaatccttataatgattatactaccttcaaccggaagttcaaggacaaggccacattgactgtacacaaatcctccagcacagcctatatggagcttagcggcctgacatctgacgactctgcagtctattactgtgcaagaacctggggtcactggtatttcgatctctggggccgtggcaccgtggtcactgtctcctca

SEQ ID NO: 10 - protein, 118 aa, synthetic construct
  REGION 1..118: note = Description of Artificial Sequence: Synthetic polypeptide
  REGION 1..118: note = Synthetic Construct
QVQLQQSEAELVRPGASVKISCKAFGYTFTNHHINWVKQRPGQGLDWIGYINPYNDYTTFNRKFKDKATLTVHKSSSTAYMELSGLTSDDSAVYYCARTWGHWYFDLWGRGTVVTVSS

SEQ ID NO: 11 - DNA, 327 bp, synthetic construct
  misc_feature 1..327: note = Description of Artificial Sequence: Synthetic polynucleotide
  misc_feature 1..327: note = 3-34 VL
  source 1..327: mol_type = other DNA
  CDS 1..327
gaaatagtgatgacgcagtctccagccaccctgtctgtgtctccaggggaaagagccaccctctcctgtagggccagtcagagtgttagcagcaacttagcctggtaccagcagaaacctggccaggctcccaggctcctcatctatgattcatccaccagggccactggtatcccagccaggttcagtggcagtgggtctgggacagacttcactctcaccatcagcagcctgcagtctgaagattttgcagtttattactgtcagcagtataataactggcctccgtggacgttcggccaagggaccaaggtggaaatcaaacgg

SEQ ID NO: 12 - protein, 109 aa, synthetic construct
  REGION 1..109: note = Description of Artificial Sequence: Synthetic polypeptide
  REGION 1..109: note = Synthetic Construct
EIVMTQSPATLSVSPGERATLSCRASQSVSSNLAWYQQKPGQAPRLLIYDSSTRATGIPARFSGSGSGTDFTLTISSLQSEDFAVYYCQQYNNWPPWTFGQGTKVEIKR

SEQ ID NO: 13 - DNA, 354 bp, synthetic construct
  misc_feature 1..354: note = Description of Artificial Sequence: Synthetic polynucleotide
  misc_feature 1..354: note = A2-32 VH
  source 1..354: mol_type = other DNA
  CDS 1..354
caggtgcagctgcaggaatcgggcccaggactggtgaagccttcggagaccctgtccctcacctgcactgtctctggcggctccatcagtggttattactggagctggatccggcagcccccagggaagggcctggagtggattggatatatctattacagtggattcaccaactacaacccctccctcaagagtcgagtcaccatatcagtagacacgtccaagaaccagttctccctgaaactgagctctgtgaccgctgcggacacggccgtgtattattgtacgagagatcactcccactactactacatggacgtctggggccaagggaccacggtcaccgtctcctca

SEQ ID NO: 14 - protein, 118 aa, synthetic construct
  REGION 1..118: note = Description of Artificial Sequence: Synthetic polypeptide
  REGION 1..118: note = Synthetic Construct
QVQLQESGPGLVKPSETLSLTCTVSGGSISGYYWSWIRQPPGKGLEWIGYIYYSGFTNYNPSLKSRVTISVDTSKNQFSLKLSSVTAADTAVYYCTRDHSHYYYMDVWGQGTTVTVSS

SEQ ID NO: 15 - DNA, 333 bp, synthetic construct
  misc_feature 1..333: note = Description of Artificial Sequence: Synthetic polynucleotide
  misc_feature 1..333: note = A2-32 VL
  source 1..333: mol_type = other DNA
  CDS 1..333
cagtctgtgctgactcagccaccctcagcgtctgggacccccgggcagagggtcaccatctcttgttctggaagcagctccaacatcggaagtaatactgtaaactggtaccagcagttcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcaggatgggatgacagcctgaatggtccggtattcggcggagggaccaagctgaccgtcctaggt

SEQ ID NO: 16 - protein, 111 aa, synthetic construct
  REGION 1..111: note = Description of Artificial Sequence: Synthetic polypeptide
  REGION 1..111: note = Synthetic Construct
QSVLTQPPSASGTPGQRVTISCSGSSSNIGSNTVNWYQQFPGTAPKLLIYSNNQRPSGVPDRFSGSKSGTSASLAISGLQSEDEADYYCAGWDDSLNGPVFGGGTKLTVLG

SEQ ID NO: 17 - DNA, 354 bp, synthetic construct
  misc_feature 1..354: note = Description of Artificial Sequence: Synthetic polynucleotide
  misc_feature 1..354: note = A2-48 VH
  source 1..354: mol_type = other DNA
  CDS 1..354
caggtgcagctgcaggagtcgggcccaggactggtgaagccttcggagaccctgtccctcacctgcactgtctctggtggctccatcagtagttactactggagctggatccggcagccccccgggaagggactggaatggattgggtatatctattacagtgggtacaccaactacaacccctccctcaagagtcgagttaacatatcagtagacacgtcccagaaccagttctccctgaagctgaattctctgaccgctgcggacacggccgtttattactgtgcgagaacacactcctattactacggtatggacgtctggggccaagggaccacggtcaccgtctcctca

SEQ ID NO: 18 - protein, 118 aa, synthetic construct
  REGION 1..118: note = Description of Artificial Sequence: Synthetic polypeptide
  REGION 1..118: note = Synthetic Construct
QVQLQESGPGLVKPSETLSLTCTVSGGSISSYYWSWIRQPPGKGLEWIGYIYYSGYTNYNPSLKSRVNISVDTSQNQFSLKLNSLTAADTAVYYCARTHSYYYGMDVWGQGTTVTVSS

SEQ ID NO: 19 - DNA, 333 bp, synthetic construct
  misc_feature 1..333: note = Description of Artificial Sequence: Synthetic polynucleotide
  misc_feature 1..333: note = A2-48 VL
  source 1..333: mol_type = other DNA
  CDS 1..333
cagtctgtgctgactcagccaccctcagcgtctgggacccccgggcagagggtcaccatctcttgttctggaagcagctccaacatcggaaataatcctgtaaactggtaccagcagttcccaggaacggcccccaaactcctcatctatagtaataatcagcggccctcaggggtccctgaccgattctctggctccaagtctggcacctcagcctccctggccatcagtgggctccagtctgaggatgaggctgattattactgtgaagcatgggatgacagcctgaatggtccggtattcggcggagggaccaagctgaccgtcctaggt

SEQ ID NO: 20 - protein, 111 aa, synthetic construct
  REGION 1..111: note = Description of Artificial Sequence: Synthetic polypeptide
  REGION 1..111: note = Synthetic Construct
QSVLTQPPSASGTPGQRVTISCSGSSSNIGNNPVNWYQQFPGTAPKLLIYSNNQRPSGVPDRFSGSKSGTSASLAISGLQSEDEADYYCEAWDDSLNGPVFGGGTKLTVLG

SEQ ID NO: 21 - DNA, 363 bp, synthetic construct
  misc_feature 1..363: note = Description of Artificial Sequence: Synthetic polynucleotide
  misc_feature 1..363: note = A2-73 VH
  source 1..363: mol_type = other DNA
  CDS 1..363
caggtgcagctgcaggagtcgggcccaggactggtgaagccttcggagaccctgtccctcacctgcactgtctctggtggctccatcagtagttactactggacctggatccggcagcccccagggaagggactggaattggttggatatatctattacagtggttacaccagctacaacccctccctcaagagtcgagtcaccatatcaattgacacgtccaagaaccagttctccctgcagctgaactctgtgaccgctgcggacacggccgtgtatttctgtgcgcgagatgactatggtggtaacggctactacggtgtggacgtctggggccaagggaccacggtcaccgtctcctca

SEQ ID NO: 22 - protein, 121 aa, synthetic construct
  REGION 1..121: note = Description of Artificial Sequence: Synthetic polypeptide
  REGION 1..121: note = Synthetic Construct
QVQLQESGPGLVKPSETLSLTCTVSGGSISSYYWTWIRQPPGKGLELVGYIYYSGYTSYNPSLKSRVTISIDTSKNQFSLQLNSVTAADTAVYFCARDDYGGNGYYGVDVWGQGTTVTVSS

SEQ ID NO: 23 - DNA, 333 bp, synthetic construct
  misc_feature 1..333: note = Description of Artificial Sequence: Synthetic polynucleotide
  misc_feature 1..333: note = A2-73 VL
  source 1..333: mol_type = other DNA
  CDS 1..333
cagtctgtgctgactcagccaccctcagcgtctgggacccccgggcagagggtcaccatctcttgttctggcagcagctccaacatcggaaataatcctgtaaactggtaccggcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaagctgaccgtcctaggt

SEQ ID NO: 24 - protein, 111 aa, synthetic construct
  REGION 1..111: note = Description of Artificial Sequence: Synthetic polypeptide
  REGION 1..111: note = Synthetic Construct
QSVLTQPPSASGTPGQRVTISCSGSSSNIGNNPVNWYRQLPGTAPKLLIYSNNQRPSGVPDRFSGSKSGTSASLAISGLQSEDEADYYCAAWDDSLNGWVFGGGTKLTVLG

SEQ ID NO: 25 - DNA, 726 bp, synthetic construct
  misc_feature 1..726: note = Description of Artificial Sequence: Synthetic polynucleotide
  misc_feature 1..726: note = 3-34-j6 scFv
  source 1..726: mol_type = other DNA
  CDS 1..726
gaagttcagctgttggaaagtggtggcggactggtccagcctggcgaatctctgagactgagctgcaaggccttcgggtatacctttaccaaccaccacatcaattgggttcgacaagcccctggcaagtgcctggaatgggtcggatatatcaacccctacaacgactacaccaccttcaacgacaagttcaaggaccggttcaccctgtccgtggataagtccaagagcaccgcttacctgcagatgaactccctgagagccgaggatacagctgtgtattactgtgcccggacctggggccactggtactttgatttgtggggccagggaaccctcgtgacagtctcttctggcggcggaggaagcggaggcggaggttcaggcggcggtggatctaacatccagatgacccagtctccatccagcctgtctgcctctgtgggcgacagagtgacaattacctgtcgggcctctcagtccgtgtccagcaatctggcttggtatcagcagaagcccggcaaggctcccaagctgctgatctacgactcctccaccagagctaccggcgtgccatctagattctccggctctggctctggcaccgactttaccctgacaatttccagcctgcagcctgaggacttcgccacctactactgccagcagtacaacaactggcctccttggaccttcggctgcggcaccaaactggaaatcaagcgc

SEQ ID NO: 26 - protein, 242 aa, synthetic construct
  REGION 1..242: note = Description of Artificial Sequence: Synthetic polypeptide
  REGION 1..242: note = Synthetic Construct
EVQLLESGGGLVQPGESLRLSCKAFGYTFTNHHINWVRQAPGKCLEWVGYINPYNDYTTFNDKFKDRFTLSVDKSKSTAYLQMNSLRAEDTAVYYCARTWGHWYFDLWGQGTLVTVSSGGGGSGGGGSGGGGSNIQMTQSPSSLSASVGDRVTITCRASQSVSSNLAWYQQKPGKAPKLLIYDSSTRATGVPSRFSGSGSGTDFTLTISSLQPEDFATYYCQQYNNWPPWTFGCGTKLEIKR

SEQ ID NO: 27 - DNA, 732 bp, synthetic construct
  misc_feature 1..732: note = Description of Artificial Sequence: Synthetic polynucleotide
  misc_feature 1..732: note = tA2-32 scFv
  source 1..732: mol_type = other DNA
  CDS 1..732
caggttcagttgcaagaatctggcccaggcctcgtgaagccctccgaaacactgtctctgacctgcacagtgtccggcggctctatctctggctactactggtcctggatcagacagcctcctggcaagtgcctggaatggatcggctacatctactactctggcttcaccaactacaaccccagcctgaagtccagagtgaccatcagcgtggacacctctaagaaccagttctctctgaagctgtcctccgtgaccgccgctgataccgctgtgtattactgcacccgggaccacagccactactactacatggacgtgtggggacagggaacaaccgtgacagtctcttcaggcggtggtggatcaggcggcggaggaagtggtggcggaggttctcagtctgtgctgacccaacctccttccgcttctggcacacctggccagagagtgacaatctcttgctccggctcctcctccaacatcggctctaacaccgtgaactggtatcagcagttccccggcaccgctcctaagctgctgatctactccaacaaccagcggccttccggcgtgcccgatagattctctggctctaagtccggcacctctgccagcctggctatttctggactgcagagcgaggacgaggccgactattattgtgccggctgggacgactctctgaacggccctgtttttggctgcggcaccaaactgactgtgctgggc

SEQ ID NO: 28 - protein, 244 aa, synthetic construct
  REGION 1..244: note = Description of Artificial Sequence: Synthetic polypeptide
  REGION 1..244: note = Synthetic Construct
QVQLQESGPGLVKPSETLSLTCTVSGGSISGYYWSWIRQPPGKCLEWIGYIYYSGFTNYNPSLKSRVTISVDTSKNQFSLKLSSVTAADTAVYYCTRDHSHYYYMDVWGQGTTVTVSSGGGGSGGGGSGGGGSQSVLTQPPSASGTPGQRVTISCSGSSSNIGSNTVNWYQQFPGTAPKLLIYSNNQRPSGVPDRFSGSKSGTSASLAISGLQSEDEADYYCAGWDDSLNGPVFGCGTKLTVLG

SEQ ID NO: 29 - DNA, 747 bp, synthetic construct
  misc_feature 1..747: note = Description of Artificial Sequence: Synthetic polynucleotide
  misc_feature 1..747: note = tA2-32LH scFv
  source 1..747: mol_type = other DNA
  CDS 1..747
caatctgttctgacccaacctccttccgcctctggcacacctggccagagagtgaccatctcttgctccggctcctcctccaacatcggctctaacaccgtgaactggtatcagcagttccccggcaccgctcctaagctgctgatctactccaacaaccagcggccttccggcgtgcccgatagattctctggctctaagtccggcacctctgccagcctggctatttctggactgcagagcgaggacgaggccgactattattgtgccggctgggacgactctctgaacggccctgtttttggcggtggcaccaaactgacagtgcttggaggcggtggtggatcaggcggcggtggaagtggtggtggtggtagcggaggtggtggatctcaggtgcagctccaagagtctggacctggcctcgtgaagccttccgagacactgtctctgacctgcacagtgtccggcggctctatctctggctactactggtcctggatcagacagcctcctggcaagggactcgagtggatcggctacatctactactctggcttcaccaactacaaccccagcctgaagtcccgcgtgaccatcagcgtggacacctctaagaaccagttctctctgaagctgtcctccgtgaccgccgctgataccgctgtgtattactgcacccgggaccacagccactactactacatggacgtgtggggacagggaacaaccgtgacagtgtcctcc

SEQ ID NO: 30 - protein, 249 aa, synthetic construct
  REGION 1..249: note = Description of Artificial Sequence: Synthetic polypeptide
  REGION 1..249: note = Synthetic Construct
QSVLTQPPSASGTPGQRVTISCSGSSSNIGSNTVNWYQQFPGTAPKLLIYSNNQRPSGVPDRFSGSKSGTSASLAISGLQSEDEADYYCAGWDDSLNGPVFGGGTKLTVLGGGGGSGGGGSGGGGSGGGGSQVQLQESGPGLVKPSETLSLTCTVSGGSISGYYWSWIRQPPGKGLEWIGYIYYSGFTNYNPSLKSRVTISVDTSKNQFSLKLSSVTAADTAVYYCTRDHSHYYYMDVWGQGTTVTVSS

SEQ ID NO: 31 - DNA, 732 bp, synthetic construct
  misc_feature 1..732: note = Description of Artificial Sequence: Synthetic polynucleotide
  misc_feature 1..732: note = tA2-48 scFv
  source 1..732: mol_type = other DNA
  CDS 1..732
caggttcagttgcaagaatctggcccaggcctcgtgaagccctccgaaacactgtctctgacctgcacagtgtccggcggctccatctcttcctactactggtcctggatcagacagcctcctggcaagtgcctggaatggatcggctacatctactactccggctacaccaactacaaccccagcctgaagtccagagtgaacatctccgtggacaccagccagaaccagttctctctgaagctgaactccctgaccgccgctgataccgctgtgtattactgcgctcggacccacagctactactatggcatggacgtgtggggacaagggacaaccgtgacagtctcttcaggcggtggtggatcaggcggcggaggaagtggtggcggaggttctcagtctgtgctgacccaacctccttccgcttctggcacacctggccagagagtgaccatcagctgtagcggctcctcctccaacatcggcaacaaccccgtgaactggtatcagcagttccccggcacagcccctaagctgctgatctactccaacaaccagcggccttccggcgtgcccgatagattctctggctctaagtccggcacctctgccagcctggctatttctggactgcagagcgaggacgaggccgactactattgcgaggcttgggacgactctctgaacggccctgtgtttggctgtggcaccaaactgactgtgctgggc

SEQ ID NO: 32 - protein, 244 aa, synthetic construct
  REGION 1..244: note = Description of Artificial Sequence: Synthetic polypeptide
  REGION 1..244: note = Synthetic Construct
QVQLQESGPGLVKPSETLSLTCTVSGGSISSYYWSWIRQPPGKCLEWIGYIYYSGYTNYNPSLKSRVNISVDTSQNQFSLKLNSLTAADTAVYYCARTHSYYYGMDVWGQGTTVTVSSGGGGSGGGGSGGGGSQSVLTQPPSASGTPGQRVTISCSGSSSNIGNNPVNWYQQFPGTAPKLLIYSNNQRPSGVPDRFSGSKSGTSASLAISGLQSEDEADYYCEAWDDSLNGPVFGCGTKLTVLG

SEQ ID NO: 33 - DNA, 741 bp, synthetic construct
  misc_feature 1..741: note = Description of Artificial Sequence: Synthetic polynucleotide
  misc_feature 1..741: note = tA2-73 scFv
  source 1..741: mol_type = other DNA
  CDS 1..741
caggttcagttgcaagaatctggcccaggcctcgtgaagccctccgaaacactgtctctgacctgcacagtgtccggcggctccatctcttcctactactggacctggatcagacagcctcctggcaagtgcttggaactcgtgggctacatctactactccggctacaccagctacaaccccagcctgaagtccagagtgaccatcagcatcgacacctctaagaaccagttcagcctgcagctcaactccgtgaccgctgctgataccgctgtgtacttctgcgccagagatgactacggcggcaacggctattatggcgtggacgtttggggacaagggacaaccgtgacagtgtctagcggaggtggtggatcaggcggcggtggaagtggtggtggtggctctcagtctgtgctgacccaacctccttctgcctctggcacacctggccagagagtgacaatctcttgctccggctcctcctccaacatcggcaacaaccccgtgaactggtacagacagctgccaggcacagcccctaagctgctgatctactccaacaaccagcggccttccggcgtgcccgatagattctctggatctaagtccggcaccagcgccagcctggctatttctggactgcagagcgaggacgaggccgactactactgtgccgcttgggacgattctctgaacggatgggtgttcggctgcggcaccaaactgactgtgctggga

SEQ ID NO: 34 - protein, 247 aa, synthetic construct
  REGION 1..247: note = Description of Artificial Sequence: Synthetic polypeptide
  REGION 1..247: note = Synthetic Construct
QVQLQESGPGLVKPSETLSLTCTVSGGSISSYYWTWIRQPPGKCLELVGYIYYSGYTSYNPSLKSRVTISIDTSKNQFSLQLNSVTAADTAVYFCARDDYGGNGYYGVDVWGQGTTVTVSSGGGGSGGGGSGGGGSQSVLTQPPSASGTPGQRVTISCSGSSSNIGNNPVNWYRQLPGTAPKLLIYSNNQRPSGVPDRFSGSKSGTSASLAISGLQSEDEADYYCAAWDDSLNGWVFGCGTKLTVLG

SEQ ID NO: 35 - DNA, 2115 bp, synthetic construct
  misc_feature 1..2115: note = Description of Artificial Sequence: Synthetic polynucleotide
  misc_feature 1..2115: note = hKM3900_3-34-j6 HC
  source 1..2115: mol_type = other DNA
  CDS 1..2115
gaagtgcagctggttcagtctggcgccgaagtgaagaaacctggcgcctccgtgaagatctcctgcaaggcttctggctacaccttcaccgactactacatgaactgggtccgacaggctcctggacagggacttgagtggatgggagatgtggtgcccaacaacggcgtgcccacctacaaccagaaattcaagggcagagtgaccatcaccgccgacaagtctacctccaccgcctacatggaactgcggagcctgagatctgaggacaccgccgtgtactactgcgccagacctcactactactacgccggcagatctggcgccatggattattggggacagggcaccctggtcaccgtgtcctctgctagcacaaagggcccctctgtgttccctctggctcctagctctaagtccacctctggcggaacagctgctctgggctgtctggtcaaggactacttccctgagcctgtgaccgtgtcttggaactctggcgctctgacatccggcgtgcacacatttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgccggcatcgaaaagaccatctccaaggccaagggccagcctagggaaccccaggtttacaccttgccaccttctcgggacgagctgaccaagaaccaggtgtccctgacctgtctcgtgaagggcttctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgcagcgtgatgcacgaggccctgcacaatcactacacacagaagtctctgtctctgagccccggcaaaggtggcggaggatctggcggaggcggatccgaagttcagctgttggaaagtggtggcggactggtccagcctggcgaatctctgagactgagctgcaaggccttcgggtatacctttaccaaccaccacatcaattgggttcgacaagcccctggcaagtgcctggaatgggtcggatatatcaacccctacaacgactacaccaccttcaacgacaagttcaaggaccggttcaccctgtccgtggataagtccaagagcaccgcttacctgcagatgaactccctgagagccgaggatacagctgtgtattactgtgcccggacctggggccactggtactttgatttgtggggccagggaaccctcgtgacagtctcttctggcggcggaggaagcggaggcggaggttcaggcggcggtggatctaacatccagatgacccagtctccatccagcctgtctgcctctgtgggcgacagagtgacaattacctgtcgggcctctcagtccgtgtccagcaatctggcttggtatcagcagaagcccggcaaggctcccaagctgctgatctacgactcctccaccagagctaccggcgtgccatctagattctccggctctggctctggcaccgactttaccctgacaatttccagcctgcagcctgaggacttcgccacctactactgccagcagtacaacaactggcctccttggaccttcggctgcggcaccaaactggaaatcaagcgc

SEQ ID NO: 36 - protein, 705 aa, synthetic construct
  REGION 1..705: note = Description of Artificial Sequence: Synthetic polypeptide
  REGION 1..705: note = Synthetic Construct
EVQLVQSGAEVKKPGASVKISCKASGYTFTDYYMNWVRQAPGQGLEWMGDVVPNNGVPTYNQKFKGRVTITADKSTSTAYMELRSLRSEDTAVYYCARPHYYYAGRSGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLLESGGGLVQPGESLRLSCKAFGYTFTNHHINWVRQAPGKCLEWVGYINPYNDYTTFNDKFKDRFTLSVDKSKSTAYLQMNSLRAEDTAVYYCARTWGHWYFDLWGQGTLVTVSSGGGGSGGGGSGGGGSNIQMTQSPSSLSASVGDRVTITCRASQSVSSNLAWYQQKPGKAPKLLIYDSSTRATGVPSRFSGSGSGTDFTLTISSLQPEDFATYYCQQYNNWPPWTFGCGTKLEIKR

SEQ ID NO: 37 - DNA, 2136 bp, synthetic construct
  misc_feature 1..2136: note = Description of Artificial Sequence: Synthetic polynucleotide
  misc_feature 1..2136: note = hKM3900_tA2-32LH HC
  source 1..2136: mol_type = other DNA
  CDS 1..2136
gaagtgcagctggttcagtctggcgccgaagtgaagaaacctggcgcctccgtgaagatctcctgcaaggcttctggctacaccttcaccgactactacatgaactgggtccgacaggctcctggacagggacttgagtggatgggagatgtggtgcccaacaacggcgtgcccacctacaaccagaaattcaagggcagagtgaccatcaccgccgacaagtctacctccaccgcctacatggaactgcggagcctgagatctgaggacaccgccgtgtactactgcgccagacctcactactactacgccggcagatctggcgccatggattattggggacagggcaccctggtcaccgtgtcctctgctagcacaaagggcccctctgtgttccctctggctcctagctctaagtccacctctggcggaacagctgctctgggctgtctggtcaaggactacttccctgagcctgtgaccgtgtcttggaactctggcgctctgacatccggcgtgcacacatttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgccggcatcgaaaagaccatctccaaggccaagggccagcctagggaaccccaggtttacaccttgccaccttctcgggacgagctgaccaagaaccaggtgtccctgacctgtctcgtgaagggcttctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgcagcgtgatgcacgaggccctgcacaatcactacacacagaagtctctgtctctgagccccggcaaaggtggcggaggatctggcggaggcggatcccaatctgttctgacccaacctccttccgcctctggcacacctggccagagagtgaccatctcttgctccggctcctcctccaacatcggctctaacaccgtgaactggtatcagcagttccccggcaccgctcctaagctgctgatctactccaacaaccagcggccttccggcgtgcccgatagattctctggctctaagtccggcacctctgccagcctggctatttctggactgcagagcgaggacgaggccgactattattgtgccggctgggacgactctctgaacggccctgtttttggcggtggcaccaaactgacagtgcttggaggcggtggtggatcaggcggcggtggaagtggtggtggtggtagcggaggtggtggatctcaggtgcagctccaagagtctggacctggcctcgtgaagccttccgagacactgtctctgacctgcacagtgtccggcggctctatctctggctactactggtcctggatcagacagcctcctggcaagggactcgagtggatcggctacatctactactctggcttcaccaactacaaccccagcctgaagtcccgcgtgaccatcagcgtggacacctctaagaaccagttctctctgaagctgtcctccgtgaccgccgctgataccgctgtgtattactgcacccgggaccacagccactactactacatggacgtgtggggacagggaacaaccgtgacagtgtcctcc

SEQ ID NO: 38 - protein, 712 aa, synthetic construct
  REGION 1..712: note = Description of Artificial Sequence: Synthetic polypeptide
  REGION 1..712: note = Synthetic Construct
EVQLVQSGAEVKKPGASVKISCKASGYTFTDYYMNWVRQAPGQGLEWMGDVVPNNGVPTYNQKFKGRVTITADKSTSTAYMELRSLRSEDTAVYYCARPHYYYAGRSGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQSVLTQPPSASGTPGQRVTISCSGSSSNIGSNTVNWYQQFPGTAPKLLIYSNNQRPSGVPDRFSGSKSGTSASLAISGLQSEDEADYYCAGWDDSLNGPVFGGGTKLTVLGGGGGSGGGGSGGGGSGGGGSQVQLQESGPGLVKPSETLSLTCTVSGGSISGYYWSWIRQPPGKGLEWIGYIYYSGFTNYNPSLKSRVTISVDTSKNQFSLKLSSVTAADTAVYYCTRDHSHYYYMDVWGQGTTVTVSS

SEQ ID NO: 39 - DNA, 645 bp, synthetic construct
  misc_feature 1..645: note = Description of Artificial Sequence: Synthetic polynucleotide
  misc_feature 1..645: note = hKM3900 LC
  source 1..645: mol_type = other DNA
  CDS 1..645
gacatccagctgacccagtctccatcctctctgtctgcctctgtgggcgacagagtgacaattacctgcaccgcctcctccaccgtgtcctctacctacctgcactggtatcagcagaagcccggcaaggctcccaagctgctgatctactccacctccaatctggcctctggcgtgccctctagattctccggatctggctctggaaccgactataccctgacaatctccagcctgcagcctgaggacttcgccacctactactgccaccagtaccacagatccccacctacctttggccagggcaccaagctggaaatcaagcgtacggtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 40 - protein, 215 aa, synthetic construct
  REGION 1..215: note = Description of Artificial Sequence: Synthetic polypeptide
  REGION 1..215: note = Synthetic Construct
DIQLTQSPSSLSASVGDRVTITCTASSTVSSTYLHWYQQKPGKAPKLLIYSTSNLASGVPSRFSGSGSGTDYTLTISSLQPEDFATYYCHQYHRSPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 486 aa, synthetic construct
  REGION 1..486: note = Description of Artificial Sequence: Synthetic polypeptide
  REGION 1..486: note = anti-CD3 scFv-human Fc fusion protein
EVQLVESGGGLIQPGGSLRLSCAASGFTFNTYAMNWVRQAPGKGLEWVARIRSKYNNYATYYADSVKDRFTISRDDSKSTLYLQMNNLRAEDTAVYYCVRHGNFGNSYVSWFAYWGQGTLVTVSSGKPGSGKPGSGKPGSGKPGSQAVVTQEPSLTVSPGGTVTLTCRSSTGAVTTSNYANWVQQKPGQAPRGLIGGTNKRAPGTPARFSGSLLGDKAALTLSGAQPEDEAEYYCALWYSNLWVFGSGTKVTVLEPKSSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 43 - protein, 254 aa, Macaca fascicularis
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 44 - protein, 6 aa, synthetic construct
  REGION 1..6: note = Description of Artificial Sequence: Synthetic peptide
  REGION 1..6: note = His tag protein
HHHHHH

SEQ ID NO: 45 - protein, 26 aa, synthetic construct
  REGION 1..26: note = Description of Artificial Sequence: Synthetic peptide
  REGION 1..26: note = 3xFLAG tag proteinE
DYKDDDDKGDYKDDDDKIDYKDDDDK

SEQ ID NO: 46 - protein, 4 aa, synthetic construct
  REGION 1..4: note = Description of Artificial Sequence: Synthetic peptide
  REGION 1..4: note = GS linker
GGGS

SEQ ID NO: 47 - protein, 4 aa, synthetic construct
  REGION 1..4: note = Description of Artificial Sequence: Synthetic peptide
  REGION 1..4: note = GS linker
SGGG

SEQ ID NO: 48 - protein, 5 aa, synthetic construct
  REGION 1..5: note = Description of Artificial Sequence: Synthetic peptide
  REGION 1..5: note = GS linker
GGGGS

SEQ ID NO: 49 - protein, 5 aa, synthetic construct
  REGION 1..5: note = Description of Artificial Sequence: Synthetic peptide
  REGION 1..5: note = GS linker
SGGGG

SEQ ID NO: 50 - protein, 6 aa, synthetic construct
  REGION 1..6: note = Description of Artificial Sequence: Synthetic peptide
  REGION 1..6: note = GS linker
GGGGGS

SEQ ID NO: 51 - protein, 6 aa, synthetic construct
  REGION 1..6: note = Description of Artificial Sequence: Synthetic peptide
  REGION 1..6: note = GS linker
SGGGGG

SEQ ID NO: 52 - protein, 7 aa, synthetic construct
  REGION 1..7: note = Description of Artificial Sequence: Synthetic peptide
  REGION 1..7: note = GS linker
GGGGGGS

SEQ ID NO: 53 - protein, 7 aa, synthetic construct
  REGION 1..7: note = Description of Artificial Sequence: Synthetic peptide
  REGION 1..7: note = GS linker
SGGGGGG

SEQ ID NO: 54 - protein, 10 aa, synthetic construct
  REGION 1..10: note = Description of Artificial Sequence: Synthetic peptide
  REGION 1..10: note = GS linker
GGGGSGGGGS

SEQ ID NO: 55 - protein, 5 aa, synthetic construct
  REGION 1..5: note = Description of Artificial Sequence: Synthetic peptide
  REGION 1..5: note = GKPGS linker
GKPGS

SEQ ID NO: 56 - protein, 5 aa, synthetic construct
  REGION 1..5: note = Description of Artificial Sequence: Synthetic peptide
  REGION 1..5: note = See specification as filed for detailed description of substitutions and preferred embodiments
GGGGS

SEQ ID NO: 57 - protein, 5 aa, synthetic construct
  REGION 1..5: note = Description of Artificial Sequence: Synthetic peptide
  REGION 1..5: note = See specification as filed for detailed description of substitutions and preferred embodiments
SGGGG

SEQ ID NO: 58 - protein, 5 aa, synthetic construct
  REGION 1..5: note = Description of Artificial Sequence: Synthetic peptide
  REGION 1..5: note = See specification as filed for detailed description of substitutions and preferred embodiments
GKPGS

SEQ ID NO: 59 - protein, 15 aa, synthetic construct
  REGION 1..15: note = Description of Artificial Sequence: Synthetic peptide
GGGGSGGGGSGGGGS

SEQ ID NO: 60 - protein, 20 aa, synthetic construct
  REGION 1..20: note = Description of Artificial Sequence: Synthetic peptide
GGGGSGGGGSGGGGSGGGGS